(19) United States
(12) Patent Application Publication
(10) Pub. No.: US 2024/0327324 A1
(43) Pub. Date: Oct. 3, 2024

(54) METHOD FOR PRODUCING FATTY ALDEHYDES AND DERIVATIVES THEREOF

(71) Applicant: FMC AGRICULTURAL SOLUTIONS A/S, Harboøre (DK)

(72) Inventors: Anders GABRIELSSON, Copenhagen (DK); Andrea MAZZIOTTA, Copenhagen (DK)

(21) Appl. No.: 18/293,550

(22) PCT Filed: Aug. 2, 2022
(86) PCT No.: PCT/EP2022/071672

§ 371 Date: Jan. 30, 2024

(30) Foreign Application Priority Data
Aug. 6, 2021 (EP) 21190097.2
Mar. 9, 2022 (EP) 22161123.9

(51) Int. Cl. C07C 45/37; B01J 31/18; B01J 31/22; C07C 45/80

(52) U.S. Cl. CPC C07C 45/37; B01J 31/181; B01J 31/1815; B01J 31/2208; C07C 45/80; B01J 2231/763; B01J 2531/004; B01J 2531/16

(57) ABSTRACT

The present invention relates to a method for converting alcohols to aldehydes, in particular fatty alcohols to fatty aldehydes, said method utilizing a catalyst, wherein the method is capable of providing high conversion of said alcohol, for example on a large scale, wherein the reaction and purification utilise a relatively small amount of solvent, and wherein the purification is capable of removing the catalyst from the product aldehyde.

### TECHNICAL FIELD

The present invention relates to a method for converting alcohols to aldehydes, in particular fatty alcohols to fatty aldehydes, said method utilizing a catalyst, wherein the method is capable of providing high conversion of said alcohol, for example on a large scale, wherein the reaction and purification utilise a relatively small amount of solvent, and wherein the purification is capable of removing the catalyst from the product aldehyde.

### BACKGROUND

The economical and sustainable oxidation of primary alcohols to aldehydes on an industrial scale is a challenging problem for the chemical industry. Although many methods are described in the literature, most of them are problematic as they utilise toxic reagents, expensive chemicals, have limited functional group tolerance, show poor yield of reaction, or require harsh conditions.

There are several oxidation protocols in the academic literature that are using an aminoxyl radical copper complex as a catalyst. The copper complex is usually created in situ by mixing a copper precursor, for example [Cu^{I}(CH₃CN)₄]⁺X⁻, where X normally is an anion, for example tetrafluoroborate, triflouromethanesulfonate, hexafluorophosphate, or a halogen with a ligand, typically 2,2′-bipyridine (BIPY). Frequently, this catalyst system also includes a base, for example 1-methyl-imidazole (MeIM). The aminoxyl radical is typically (2,2,6,6-tetramethylpiperidin-1-yl) oxyl (TEMPO) or a derivative thereof such as (4-hydroxy-2,2,6,6-tetramethylpiperidin-1-yl)oxyl (4-OH-TEMPO). The aminoxyl radical can also be generated in situ from a hydroxyl amine or an oxoammonium salt. This catalyst system is reported to achieve nearly quantitative yield of aldehyde when a primary alcohol is oxidized with molecular oxygen. However, these reactions are normally conducted in small scale with large amount of solvents (i.e. at low concentration of substrate) and expensive purification. When applying those methods to industrial feedstocks, such as complex mixtures of alcohols, and more concentrated solution, it has not been possible to achieve good yields and selectivity of reaction. It is likewise difficult removing/reclaiming the catalyst from the reaction medium in a cost-effective manner.

A typical procedure for oxidation of alcohol with subsequent removal of the catalyst component is described in by Stahl and co-workers (J. Am. Chem. Soc. 2011, 133, 16901-16910). The procedure is carried out on a scale of 1 mmol of the alcohol. While this procedure provides acceptable results in the lab, it is not applicable to large-scale production of fatty aldehydes because the high number of steps as well as the need for column chromatography gives a prohibitively expensive catalyst removal. Furthermore, the solvent volume of 60 ml dichloromethane per ml reaction mixture further adds to the costs.

Kumpulainen and Ari M. P. Koskinen have published a procedure that does not require column chromatography (Chem. Eur. J. 2009, 15, 10901-10911). The method is carried out on a scale of 10 mmol of the alcohol (1-decanol, 1.58 g). However, the method requires numerous steps and a large volume of solvent, making this procedure infeasible for industrialisation.

Norman Lui et. al. Tetrahedron Letters 48 (2007) 8823-8828 relates to novel ligands and CuBr TEMPO for oxidation under Flourous Biphasic conditions and Thermomorphic Mode.

Hoover et. al J. Am. Chem. Soc. 2011, 133, 42, 16901-16910 relates to Cu/TEMPO catalyst systems for aerobic oxidation of primary alcohols.

Steves et. al. J. Am. Chem. Soc. 2013, 135, 42, 15742-15745 relates to Cu/TEMPO catalyst systems for aerobic oxidation of sterically unhindered primary alcohols.

Wei et. Al. Green Chem., 2019, 21, 4069 relates to oxidation of alcohols to aldehydes or ketones using inorganic-ligand supported copper catalyst.

U.S. Pat. No. 5,155,280A1 relates to preparation of an aldehyde which comprises reacting the corresponding alkanol with a solubilized stable free radical nitroxide.

The large number of steps involved in above-mentioned procedures is also an issue as each step is associated with loss of product. The loss is further increased if the conversion is carried out at higher concentration. Furthermore, while acid used during work-up is moderately effective in removing the basic components of the reaction mixture (ligands and added bases), it does not remove the aminoxyl radical commonly used such as 4-Hydroxy-TEMPO or TEMPO, which is very soluble in the fatty aldehyde product mixture. A further disadvantage with most published methods is that a strong acid (e.g. sulphuric acid) is used to remove the copper. The acid is known to cause side reaction reducing the overall purity of the product.

For a feedstock of a mixture of pheromone alcohols, the present inventors have observed that the catalyst deactivates rapidly. This limits the concentration and the amount of the desired aldehyde in the product, and thus necessitates expensive and complex purification such as distillation or chromatography, which is not feasible on an industrial scale.

Accordingly, there is an unmet need for novel methods to convert primary alcohols, such as for instance fatty alcohols, to the corresponding aldehydes, e.g. fatty aldehydes. To meet this need, the method must be scalable and be applicable to feedstock mixtures.

### SUMMARY

The present inventors have discovered a convenient method of converting an alcohol composition to an aldehyde composition. The method utilises a relatively small amount of solvent, is scalable, to industrial scale, even to 100 kilogram batch size or more and provides for a product of high purity, in particular with respect to removal of catalyst composition. The method is especially suitable for conversion of a fatty alcohol composition to the corresponding fatty aldehyde composition, because it provides for a high degree of conversion of the fatty alcohol composition. Further, the method described herein has the advantage that it limits the competing reaction of oxidizing the alcohol to the corresponding acid and thus the method provides for a conversion of alcohol into aldehyde which is significantly higher that the conversion of alcohol into acid.

In one aspect the present disclosure provides for a method of converting a fatty alcohol to a fatty aldehyde, said method comprising the steps of:
a) providing a reaction mixture comprising a fatty alcohol, a catalyst comprising a copper source, and a solvent, and
b) oxidizing the fatty alcohol by adding O₂ to the reaction mixture in an amount sufficient for converting more than 50 wt % of the fatty alcohol to fatty aldehyde and less than 50 wt % into fatty acid.

In a further aspect, the present disclosure provides for a method for large scale conversion of a fatty alcohol into a fatty aldehyde, said method comprising the steps of:
a) providing a reaction mixture comprising at least 1 kilogram of fatty alcohol, a catalyst comprising a copper source, at least 1 kilogram of solvent, and a water absorbing or adsorbing material absorbing or adsorbing water, and
b) dissolving at least 0.01 μmol O₂ per minute per μmol copper in the reaction mixture or at least 0.001 μmol O₂ per minute per μmol initial fatty alcohol in the reaction mixture to the reaction mixture by feeding a gas or a liquid comprising O₂ into the reaction medium and thereby oxidizing more than 50 wt % of the fatty alcohol into fatty aldehyde and less than 50 wt % into fatty acid.

In a further aspect, the present disclosure provides for a method for conversion of a fatty alcohol into a fatty aldehyde, said method comprising the steps of:
a) providing a reaction mixture comprising at least 1 kilogram of fatty alcohol, a catalyst comprising a copper source, at least 1 kilogram of solvent, and a water absorbing or adsorbing material absorbing or adsorbing water, and
b) dissolving at least 0.01 μmol O₂ per minute per μmol copper in the reaction mixture or at least 0.001 μmol O₂ per minute per μmol initial fatty alcohol in the reaction mixture to the reaction mixture by feeding a gas or a liquid comprising Oz into the reaction medium and thereby oxidizing more than 50 wt % of the fatty alcohol into fatty aldehyde and less than 50 wt % into fatty acid.

In another aspect of the present disclosure provides for a method of converting an alcohol to an aldehyde, said method comprising the steps of:
a. providing a reaction mixture comprising an alcohol composition comprising the alcohol, a catalyst composition as disclosed herein, and a solvent as disclosed herein, and
b. exposing the reaction mixture to an oxygen flow as disclosed herein by means of bubbling a gas mixture comprising oxygen through the reaction mixture, thereby obtaining the aldehyde.

One embodiment of the present disclosure provides for a fatty aldehyde purification method comprising the steps of:
a. providing a crude reaction product comprising:
   i. a fatty aldehyde,
   ii. copper ions, and
   iii. a polar solvent;
b. mixing said crude reaction product with an apolar, aprotic solvent and an acid to create an apolar phase and a polar phase; and
c. separating the apolar phase from the polar phase.

One aspect of the present disclosure provides for an aldehyde composition obtained from a method comprising the steps of:
a. providing a reaction mixture comprising an alcohol composition comprising the alcohol, a catalyst composition as disclosed herein, and a solvent as disclosed herein, and
b. exposing the reaction mixture to an oxygen flow as disclosed herein by means of bubbling a gas mixture comprising oxygen through the reaction mixture, thereby obtaining the aldehyde composition.

One aspect of the present disclosure provides for a method of converting an alcohol to an acetal, said method comprising the steps of:
a. providing a reaction mixture comprising an alcohol composition comprising the alcohol, a catalyst composition as disclosed herein, and a solvent as disclosed herein,
b. exposing the reaction mixture to an oxygen flow as disclosed herein by means of bubbling a gas mixture comprising oxygen through the reaction mixture, thereby obtaining an aldehyde, and
c. converting the aldehyde functional groups of the aldehyde to acetal functional groups, thereby obtaining the acetal.

One aspect of the present disclosure provides for a method of converting an alcohol to an α-hydroxysulfonic acid, said method comprising the steps of:
a. providing a reaction mixture comprising an alcohol composition comprising the alcohol, a catalyst composition as disclosed herein, and a solvent as disclosed herein,
b. exposing the reaction mixture to an oxygen flow as disclosed herein by means of bubbling a gas mixture comprising oxygen through the reaction mixture, thereby obtaining a aldehyde, and
c. converting the aldehyde functional groups of the aldehyde to α-hydroxysulfonic acid functional groups,
   thereby obtaining the α-hydroxysulfonic acid.

One aspect of the present disclosure provides for a pheromone component produced from renewable feedstocks, said pheromone component having at least than 80% of biobased carbon content.

In a further aspect the present disclosure provides for a composition comprising more than 93% by weight fatty aldehyde, less than 7% by weight fatty alcohol and less than 2% by weight water.

In a further aspect, a composition is provided comprising more than 93% by weight fatty aldehyde, less than 7% by weight fatty alcohol and less than 2% by weight water.

### DESCRIPTION OF DRAWINGS

FIG. 1: Conversion of fatty alcohol to fatty aldehyde at high oxygen transfer rate. The reaction was left for 2 h during which the temperature increased from 22° C. to 52° C. after 1 h followed by a drop in temperature to 42° C. after 2 h. The reaction yield increased steadily to over 70% after 73 min, and increased further to 87% at 150 min.

FIG. 2: Conversion of fatty alcohol to fatty aldehyde at high oxygen transfer rate and in the presence of water adsorbent. The reaction was left for 2 h during which the temperature increased from 22° C. to 52° C. after 1 h followed by a drop in temperature to 42° C. after 2 h. The conversion increased steadily to over 95% at 139 min.

FIG. 3: Conversion of fatty alcohol to fatty aldehyde at very high oxygen transfer rate and in the presence of water adsorbent. The reaction was left for 2 h during which the temperature increased from 23° C. to 51° C. after 1 and 13 min followed by a drop in temperature to 22° C. after 6 h. The conversion increased steadily to over 99% at 110 min.

FIG. 4: Oxidation of Fatty alcohol mixture in 4 m³ reactor as described in Example 16. FIG. 4 shows the reaction data.

FIG. 5: Oxidation of Fatty alcohol mixture in 4 m³ reactor as described in Example 16. FIG. 5 shows the conversion over time.

FIG. 6: Oxidation of Fatty alcohol mixture in 4 m³ reactor as described in Example 17. FIG. 6 shows the reaction data of the oxidation process.

FIG. 7: Oxidation of Fatty alcohol mixture in 4 m³ reactor as described in Example 17. FIG. 7 shows the conversion over time.

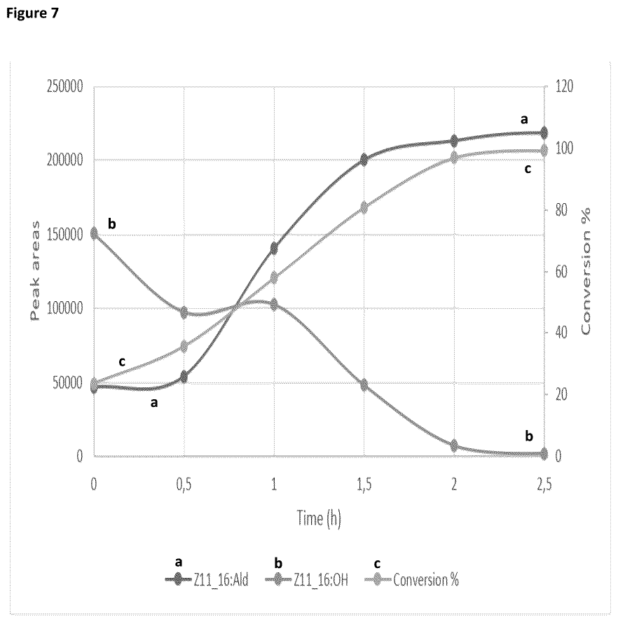

### DETAILED DESCRIPTION

### Definitions

Terms such as “X comprises in the range of n to m of Y” as used herein refer to that X contains at least n and at the most m of Y. I.e. the term indicates that X does not contain less than n of Y and does not contain more than m of Y. By way of example, if a composition is stated to comprise in the range of 5 to 60% of Y, then said composition does not contain less than 5% of Y and does not contain more than 60% of Y.

As used herein, the singular terms “a” and “the” are synonymous and used interchangeably with “one or more” and “at least one”, unless the language and/or context clearly indicates otherwise. Accordingly, for example, reference to “a solvent” or “the solvent” herein or in the appended claims can refer to a single solvent or more than one solvent.

“Solvent” as used herein includes a liquid that can dissolve or substantially disperse another substance.

Unless explicitly stated otherwise, references to “fatty alcohol” or “fatty aldehyde” is taken to cover both singular and plural versions of the terms. By way of example, a “composition comprising 50 wt % fatty alcohol” can comprise either a single fatty alcohol in an amount equal to 50 wt % of the composition, or it can comprise a mixture of two or more fatty alcohols in an amount equal to 50 wt % of the composition.

The terms “unsaturated” and “desaturated” are used synonymously when describing compounds having carbon-carbon double bonds. The following nomenclature is used herein throughout: a Δi desaturated compound, where i is an integer, refers to a compound having a double or triple carbon-carbon bond between the carbon atom at position i of the carbon chain, and the carbon atom at position i+1 of the carbon chain. The carbon chain length is thus at least equal to i+1. For example, a Δ12 desaturated compound refers to a compound having a double or triple carbon-carbon bond between carbon 12 and carbon 13, and is herein referred to as a carbon chain having a carbon-carbon bond at position 12. Said Δ12 desaturated compound can have a carbon chain length of 13 or more. The double or triple bond can be in an E configuration or in a Z configuration. Thus, herein, an Ei or a Zi desaturated compound will refer to a compound having a double carbon-carbon bond in an E configuration or in a Z configuration, respectively, between carbon i and carbon i+1 of the carbon chain, wherein said desaturated compound has a total length at least equal to i+1. For example, an E12 desaturated fatty alcohol has a desaturation at position 12 (i.e. a double bond between carbon atom 12 and carbon atom 13) in an E configuration, and has a carbon chain length of 13 or more.

Furthermore, as used herein, terms such as “(E)7,(Z)9”, “(E7),(Z9)”, “E7,Z9”, “(E7,Z9)”, “(7E,9Z)”, “(7E),(9Z)”, “(7)E,(9)Z” and other variations thereof are synonymous. That is, when specifying the stereochemistry of double bonds in carbon chains, parentheses may be written around terms or part of terms separately or around the entire group of stereochemical descriptors, or combinations thereof, or the parenthesis can be omitted entirely, and the position may be given either before or after the descriptor. This applies to any combination of any number of stereochemical descriptors used herein.

As used herein, the terms “chain length” or “carbon chain length” refers to the number of consecutive carbon atoms in a molecule. By way of example, the molecule hexadecan-1-ol has a chain length of 16.

Unless otherwise specified, a reference to a position in an organic molecule by means of the number of the position is based on numbering the organic molecule from the functional group, e.g. by designating the carbon atom on which the hydroxy group of the primary alcohol is attached as carbon atom 1, or by designating the carbon atom forming part of the carbonyl group of an aldehyde as carbon atom 1.

Cloud point: The cloud point of a surfactant, in particular non-ionic, or a glycol solution, in a solution, for example an aqueous solution, is the temperature at which a mixture of said surfactant and said solution, for example said aqueous solution, starts to phase-separate, and two phases appear, thus becoming cloudy. This behaviour is characteristic of non-ionic surfactants containing polyoxyethylene chains, which exhibit reverse solubility versus temperature behaviour in water and therefore “cloud out” at some point as the temperature is raised. Glycols demonstrating this behaviour are known as “cloud-point glycols”. The cloud point is affected by salinity, being generally lower in more saline fluids.

Cloud concentration: the term will herein be used to refer to the concentration of a surfactant, in particular non-ionic, or a glycol solution, in a solution above which, at a given temperature, a mixture of said surfactant and said solution starts to phase-separate, and two phases appear, thus becoming cloudy. For example, the cloud concentration of a surfactant in an aqueous solution at a given temperature is the minimal concentration of said surfactant which, when mixed with the aqueous solution, gives rise to two phases. The cloud concentration can be obtained from the manufacturer of the surfactant, or it may be determined experimentally, by making a dosage curve and determining the concentration at which the mixture phase separates.

As used herein, by “X % Y”, where Y is a gas, is meant a gas or air mixture wherein the Y question constitutes a partial pressure that is X % of the total pressure of the gas or air mixture. By way of example, a gas mixture consisting of oxygen at a partial pressure of 0.2 bar and nitrogen at a partial pressure of 0.8 bar is referred to as being “20% oxygen” or “a 20% oxygen gas mixture”.

As used herein, any reference to a volume of gas can mean said volume of pure gas, or a larger volume of a mixture of gasses comprising said gas. By way of example, “1.5 ml of oxygen” can mean either 1.5 ml of pure oxygen or 7.5 ml of a mixture of gasses comprising 20% oxygen.

Unless otherwise specified, any reference to a volume of a gas is to be considered at a pressure of 1.00 bar.

As used herein in the context of gasses, by any reference to “oxygen” is meant O₂.

As used herein, the term “alcohol” comprises the term “fatty alcohol”. As used herein, the term “alcohol composition” comprises the term “fatty alcohol composition”.

As used herein, the term “aldehyde” comprises the term “fatty aldehyde”. As used herein, the term “aldehyde composition” comprises the term “fatty aldehyde composition”.

As used herein, the term “acetal” comprises the term “fatty acetal”. As used herein, the term “acetal composition” comprises the term “fatty acetal composition”.

As used herein, the term “α-hydroxysulfonic acid” comprises the term “fatty α-hydroxysulfonic acid”. As used herein, the term “α-hydroxysulfonic acid composition” comprises the term “fatty α-hydroxysulfonic acid composition”.

The unit ppm as used herein is based on weight, unless otherwise specified.

As used herein, the term “spent” relates to an oxidizing agent which has already acted as an oxidizing agent. By way of example, O₂ is an oxidizing agent, whereas H₂O is its corresponding spent oxidizing agent. By way of example, the compound TEMPO is an oxidizing agent, whereas the compound N-hydroxy-2,2,6,6-tetramethylpiperidin is its corresponding spent oxidizing agent. Spent oxidizing agents may also be referred to as depleted oxidizing agents. A spent oxidizing agent is often a reduced form of the corresponding oxidizing agent.

### Fatty Alcohols

The present disclosure relates to fatty alcohol compositions comprising at least one fatty alcohol. In one embodiment of the disclosure, the fatty alcohol composition consists of or comprises a single fatty alcohol. In another embodiment, the fatty alcohol composition consists of or comprises a mixture of a few fatty alcohols, such as 2 to 5 fatty alcohols, i.e. 2, 3, 4 or 5 fatty alcohols. In yet another embodiment, the fatty alcohol composition consists of or comprises several fatty alcohols, such as 6 or more fatty alcohols.

In a preferred embodiment of the disclosure, the fatty alcohol is a primary fatty alcohol. Specifically, in one embodiment of the disclosure, the conversion of fatty alcohol to fatty aldehyde as disclosed herein is the conversion of a primary alcohol functional group to an aldehyde functional group. In a preferred embodiment of the disclosure, the conversion is oxidation of a primary alcohol functional group to an aldehyde functional group.

It is considered that many different primary alcohols can be converted to the corresponding aldehydes using the methods disclosed herein. The methods are especially suitable for conversion of fatty alcohols to the corresponding fatty aldehydes because other known methods either produce incomplete conversion, produce undesired by-products, and/or require a large amount of solvent.

The fatty alcohol may be a saturated fatty alcohol, a desaturated fatty alcohol. In one embodiment of the disclosure, the fatty alcohol composition comprises solely saturated fatty alcohols. In another embodiment, the fatty alcohol composition comprises solely desaturated fatty alcohols. In yet another embodiment of the present disclosure, the alcohol composition comprises both saturated and desaturated fatty alcohols.

In one embodiment, the fatty alcohol has a chain length of 8. In another embodiment, the fatty alcohol has a chain length of 9. In another embodiment, the fatty alcohol has a chain length of 10. In another embodiment, the fatty alcohol has a chain length of 11. In another embodiment, the fatty alcohol has a chain length of 12. In another embodiment, the fatty alcohol has a chain length of 13. In another embodiment, the fatty alcohol has a chain length of 14. In another embodiment, the fatty alcohol has a chain length of 15. In another embodiment, the fatty alcohol has a chain length of 16. In another embodiment, the fatty alcohol has a chain length of 17. In another embodiment, the fatty alcohol has a chain length of 18. In another embodiment, the fatty alcohol has a chain length of 19. In another embodiment, the fatty alcohol has a chain length of 20. In another embodiment, the fatty alcohol has a chain length of 21. In another embodiment, the fatty alcohol has a chain length of 22.

Fatty alcohols may be branched or unbranched (i.e. linear or “straight-chain”). In a preferred embodiment of the present disclosure, the fatty alcohol is unbranched.

In a preferred embodiment of the disclosure, the fatty alcohol has a chain length of 12 to 16. In a further embodiment of the disclosure, the fatty alcohol is unbranched and has a chain length of 12 to 16. In an even more preferred embodiment of the disclosure, the fatty alcohol is unbranched and has a chain length of 12. In another even more preferred embodiment, the fatty alcohol is unbranched and has a chain length of 14. In another even more preferred embodiment, the fatty alcohol is unbranched and has a chain length of 16.

In one embodiment of the present disclosure, the fatty alcohol is a saturated fatty alcohol. In one embodiment of the disclosure, the fatty alcohol is a saturated fatty alcohol having a carbon chain length of 8, 9, 10, 11, 12, 13, 14, 15, 16, 17, 18, 19, 20, 21, or 22.

In one embodiment of the present disclosure, the fatty alcohol is a desaturated fatty alcohol. The double bond of the desaturated fatty alcohol may have either E or Z configuration, except if the double bond is a terminal double bond. In one embodiment of the disclosure, the fatty alcohol comprises one or more E configured double bonds. In one embodiment of the disclosure, the fatty alcohol comprises one or more Z configured double bonds. In yet another embodiment, the fatty alcohol comprises one or more E configured double bonds and one or more Z configured double bonds.

In some embodiments, the fatty alcohol is a desaturated fatty alcohol. Such compounds are naturally produced e.g. by insect cells, where they act as pheromones. The desaturated fatty alcohols may be:
(Z)-Δ3 desaturated fatty alcohols having a carbon chain length of 8, 9, 10, 11, 12, 13, 14, 15, 16, 17, 18, 19, 20, 21 or 22;
(E)-Δ3 desaturated fatty alcohols having a carbon chain length of 8, 9, 10, 11, 12, 13, 14, 15, 16, 17, 18, 19, 20, 21 or 22;
(Z)-Δ5 desaturated fatty alcohols having a carbon chain length of 8, 9, 10, 11, 12, 13, 14, 15, 16, 17, 18, 19, 20, 21 or 22;
(E)-Δ5 desaturated fatty alcohols having a carbon chain length of 8, 9, 10, 11, 12, 13, 14, 15, 16, 17, 18, 19, 20, 21 or 22;
(Z)-Δ6 desaturated fatty alcohols having a carbon chain length of 8, 9, 10, 11, 12, 13, 14, 15, 16, 17, 18, 19, 20, 21 or 22;
(E)-Δ6 desaturated fatty alcohols having a carbon chain length of 8, 9, 10, 11, 12, 13, 14, 15, 16, 17, 18, 19, 20, 21 or 22;
(Z)-Δ7 desaturated fatty alcohols having a carbon chain length of 8, 9, 10, 11, 12, 13, 14, 15, 16, 17, 18, 19, 20, 21 or 22;
(E)-Δ7 desaturated fatty alcohols having a carbon chain length of 8, 9, 10, 11, 12, 13, 14, 15, 16, 17, 18, 19, 20, 21 or 22;
(Z)-Δ8 desaturated fatty alcohols having a carbon chain length of 9, 10, 11, 12, 13, 14, 15, 16, 17, 18, 19, 20, 21 or 22;
(E)-Δ8 desaturated fatty alcohols having a carbon chain length of 9, 10, 11, 12, 13, 14, 15, 16, 17, 18, 19, 20, 21 or 22;
(Z)-Δ9 desaturated fatty alcohols having a carbon chain length of 10, 11, 12, 13, 14, 15, 16, 17, 18, 19, 20, 21 or 22;
(E)-Δ9 desaturated fatty alcohols having a carbon chain length of 10, 11, 12, 13, 14, 15, 16, 17, 18, 19, 20, 21 or 22;
(Z)-Δ10 desaturated fatty alcohols having a carbon chain length of 11, 12, 13, 14, 15, 16, 17, 18, 19, 20, 21 or 22;
(E)-Δ10 desaturated fatty alcohols having a carbon chain length of 11, 12, 13, 14, 15, 16, 17, 18, 19, 20, 21 or 22;
(Z)-Δ11 desaturated fatty alcohols having a carbon chain length of 12, 13, 14, 15, 16, 17, 18, 19, 20, 21 or 22;
(E)-Δ11 desaturated fatty alcohols having a carbon chain length of 12, 13, 14, 15, 16, 17, 18, 19, 20, 21 or 22;
(Z)-Δ12 desaturated fatty alcohols having a carbon chain length of 13, 14, 15, 16, 17, 18, 19, 20, 21 or 22;
(E)-Δ12 desaturated fatty alcohols having a carbon chain length of 13, 14, 15, 16, 17, 18, 19, 20, 21 or 22;
(Z)-Δ13 desaturated fatty alcohols having a carbon chain length of 14, 15, 16, 17, 18, 19, 20, 21 or 22; and
(E)-Δ13 desaturated fatty alcohols having a carbon chain length of 14, 15, 16, 17, 18, 19, 20, 21 or 22.

In some embodiments, the fatty alcohols are desaturated fatty alcohols having a carbon chain length of 12, such as:
(Z)-Δ5 desaturated fatty alcohols having a carbon chain length of 12;
(E)-Δ5 desaturated fatty alcohols having a carbon chain length of 12;
(Z)-Δ6 desaturated fatty alcohols having a carbon chain length of 12;
(E)-Δ6 desaturated fatty alcohols having a carbon chain length of 12;
(Z)-Δ7 desaturated fatty alcohols having a carbon chain length of 12;
(E)-Δ7 desaturated fatty alcohols having a carbon chain length of 12;
(Z)-Δ8 desaturated fatty alcohols having a carbon chain length of 12;
(E)-Δ8 desaturated fatty alcohols having a carbon chain length of 12;
(Z)-Δ9 desaturated fatty alcohols having a carbon chain length of 12;
(E)-Δ9 desaturated fatty alcohols having a carbon chain length of 12;
(Z)-Δ10 desaturated fatty alcohols having a carbon chain length of 12;
(E)-Δ10 desaturated fatty alcohols having a carbon chain length of 12;
(Z)-Δ11 desaturated fatty alcohols having a carbon chain length of 12; and
(E)-Δ11 desaturated fatty alcohols having a carbon chain length of 12.

In some embodiments, the fatty alcohols are desaturated fatty alcohols having a carbon chain length of 14, such as:
(Z)-Δ5 desaturated fatty alcohols having a carbon chain length of 14;
(E)-Δ5 desaturated fatty alcohols having a carbon chain length of 14;
(Z)-Δ6 desaturated fatty alcohols having a carbon chain length of 14;
(E)-Δ6 desaturated fatty alcohols having a carbon chain length of 14;
(Z)-Δ7 desaturated fatty alcohols having a carbon chain length of 14;
(E)-Δ7 desaturated fatty alcohols having a carbon chain length of 14;
(Z)-Δ8 desaturated fatty alcohols having a carbon chain length of 14;
(E)-Δ8 desaturated fatty alcohols having a carbon chain length of 14;
(Z)-Δ9 desaturated fatty alcohols having a carbon chain length of 14;
(E)-Δ9 desaturated fatty alcohols having a carbon chain length of 14;
(Z)-Δ10 desaturated fatty alcohols having a carbon chain length of 14;
(E)-Δ10 desaturated fatty alcohols having a carbon chain length of 14;
(Z)-Δ11 desaturated fatty alcohols having a carbon chain length of 14;
(E)-Δ11 desaturated fatty alcohols having a carbon chain length of 14;
(Z)-Δ12 desaturated fatty alcohols having a carbon chain length of 14;
(E)-Δ12 desaturated fatty alcohols having a carbon chain length of 14;
(Z)-Δ13 desaturated fatty alcohols having a carbon chain length of 14; and
(E)-Δ13 desaturated fatty alcohols having a carbon chain length of 14.

In some embodiments, the fatty alcohols are desaturated fatty alcohols having a carbon chain length of 16, such as:
(Z)-Δ5 desaturated fatty alcohols having a carbon chain length of 16;
(E)-Δ5 desaturated fatty alcohols having a carbon chain length of 16;
(Z)-Δ6 desaturated fatty alcohols having a carbon chain length of 16;
(E)-Δ6 desaturated fatty alcohols having a carbon chain length of 16;
(Z)-Δ7 desaturated fatty alcohols having a carbon chain length of 16;
(E)-Δ7 desaturated fatty alcohols having a carbon chain length of 16;
(Z)-Δ8 desaturated fatty alcohols having a carbon chain length of 16;
(E)-Δ8 desaturated fatty alcohols having a carbon chain length of 16;
(Z)-Δ9 desaturated fatty alcohols having a carbon chain length of 16;
(E)-Δ9 desaturated fatty alcohols having a carbon chain length of 16;
(Z)-Δ10 desaturated fatty alcohols having a carbon chain length of 16;
(E)-Δ10 desaturated fatty alcohols having a carbon chain length of 16;
(Z)-Δ11 desaturated fatty alcohols having a carbon chain length of 16;
(E)-Δ11 desaturated fatty alcohols having a carbon chain length of 16;
(Z)-Δ12 desaturated fatty alcohols having a carbon chain length of 16;
(E)-Δ12 desaturated fatty alcohols having a carbon chain length of 16;
(Z)-Δ13 desaturated fatty alcohols having a carbon chain length of 16; and
(E)-Δ13 desaturated fatty alcohols having a carbon chain length of 16.

For example, the fatty alcohol is an (E)7,(Z)9 desaturated fatty alcohol having a carbon chain length of 10, 11, 12, 13, 14, 15, 16, 17, 18, 19, 20, 21 or 22. In some embodiments, the fatty alcohol is an (E)3, (Z)8, (Z)11 desaturated fatty alcohol having a carbon chain length of 12, 13, 14, 15, 16, 17, 18, 19, 20, 21 or 22, for example 14. In some embodiments, the fatty alcohol is a (Z)9, (E)11, (E)13 desaturated fatty alcohol having a carbon chain length of 14, 15, 16, 17, 18, 19, 20, 21 or 22. In some embodiments, the fatty alcohol is a (Z)11, (Z)13 desaturated fatty alcohol having a carbon chain length of 14, 15, 16, 17, 18, 19, 20, 21 or 22. In some embodiments, the fatty alcohol is a (Z)9, (E)12 desaturated fatty alcohol having a carbon chain length of 13, 14, 15, 16, 17, 18, 19, 20, 21 or 22. In some embodiments, the fatty alcohol is a (E)7, (E)9 desaturated fatty alcohol having a carbon chain length of 10, 11, 12, 13, 14, 15, 16, 17, 18, 19, 20, 21 or 22. In some embodiment, the fatty alcohol is a (E)8, (E)10 desaturated fatty alcohol having a carbon chain length of 11, 12, 13, 14, 15, 16, 17, 18, 19, 20, 21, or 22

In other embodiments, the fatty alcohol is an (E)7,(Z)9 desaturated fatty alcohol having a carbon chain length of 14. In other embodiments, the desaturated fatty alcohol is an (E)3, (Z)8, (Z)11 desaturated fatty alcohol having a carbon chain length of 14. In other embodiments, the desaturated fatty alcohol is a (Z)9, (E)11, (E)13 desaturated fatty alcohol having a carbon chain length of 14. For example, the fatty alcohol is an (E)7,(Z)9 desaturated fatty alcohol having a carbon chain length of 12. In other embodiments, the desaturated fatty alcohol is an (E)3, (Z)8, (Z)11 desaturated fatty alcohol having a carbon chain length of 12. In other embodiments, the desaturated fatty alcohol is a (Z)9, (E)11, (E)13 desaturated fatty alcohol having a carbon chain length of 12. In other embodiments, the desaturated fatty alcohol is a (E)8, (E)10 desaturated fatty alcohol having a carbon chain length of 12. In other embodiments, the desaturated fatty alcohol is a (E)7, (E)9 desaturated fatty alcohol having a carbon chain length of 11. In other embodiments, the desaturated fatty alcohol is a (Z)11, (Z)13 desaturated fatty alcohol having a carbon chain length of 16. In other embodiments, the desaturated fatty alcohol is a (Z)9, (E)12 desaturated fatty alcohol having a carbon chain length of 14.

In some embodiments the fatty alcohol is (Z9, E12)-tetradecadien-1-ol. Microbial cell factories and methods for obtaining (Z9, E12)-tetradecadien-1-ol from a yeast cell are described in detail in application EP21183447.8 entitled “Methods and yeast cells for production of desaturated compounds” filed on 2 Jul. 2021 by same applicant.

In some embodiments the fatty alcohol is (Z11, Z13)-hexadecadien-1-ol. Microbial cell factories and methods for obtaining (Z11, Z13)-hexadecadien-1-ol from a yeast cell are described in detail in application EP21183459.3 entitled “Methods and yeast cells for production of desaturated compounds” filed on 2 Jul. 2021 by same applicant.

In some embodiments the fatty alcohol is (E8,E10)-dodecadien-1-ol. Microbial cell factories and methods for obtaining (E8,E10)-hexadecadien-1-ol from a yeast cell are described in detail in application WO 2021/123128.

In some embodiments the fatty alcohol is (Z11)-hexadecen-1-ol. Microbial cell factories and methods for obtaining (Z11)-hexadecen-1-ol from a yeast cell are described in detail in application WO 2016/207339.

In a preferred embodiment of the disclosure, the fatty alcohol has a double bond at position 9, 11, or 13, or double bonds at positions 9 and 11 or at positions 11 and 13; or the fatty alcohol has a double bond at position 9 or 12, or double bonds at positions 9 and 12. In an even more preferred embodiment of the disclosure, the fatty alcohol has a chain length of 12 and a double bond at position 9 or 11, or double bonds at positions 9 and 11; or the fatty alcohol has a chain length of 14 and a double bond at position 9 or 12, or double bonds at positions 9 and 12; or the fatty alcohol has a chain length of 14 and a double bond at position 9, 11, or 13, or double bounds at positions 9 and 11, or at positions 11 and 13. In another more preferred embodiment of the disclosure, the fatty alcohol has a chain length of 14 and a double bond at position 9 or 11, or double bonds at positions 9 and 11. In another more preferred embodiment of the disclosure, the fatty alcohol has a chain length of 16 and a double bond at position 9 or 11, or double bonds at positions 9 and 11. In other embodiments, the fatty alcohol has a chain length of 16 and a double bond at position 11 or 13, or double bonds at positions 11 and 13. In other embodiments, the fatty alcohol has a chain length of 12 and a double bond at position 8 or 10, or double bonds at positions 8 and 10.

In a specific embodiment, the fatty alcohol is selected from the group consisting of tetradecan-1-ol, pentadecan-1-ol, hexadecan-1-ol, pentadecen-1-ol, (Z)-9-hexadecen-1-ol, (Z)-11-hexadecen-1-ol, (7E,9E)-undeca-7,9-dien-1-ol, (11Z, 13Z)-hexadecadien-1-ol, (9Z, 12E)-tetradecadien-1-ol, and (8E,10E)-dodecadien-1-ol. In a particular embodiment, the fatty alcohol is (Z)-11-hexadecen-1-ol or (Z)-9-tetradecen-1-ol.

The fatty alcohol composition may consist entirely of fatty alcohols, or it may comprise fatty alcohols and other compounds. In one embodiment of the disclosure, the fatty alcohol composition comprises 5 to 10 wt % of one or more fatty alcohols. In another embodiment, the fatty alcohol composition comprises 10 to 20 wt % of one or more fatty alcohols. In another embodiment, the fatty alcohol composition comprises 20 to 30 wt % of one or more fatty alcohols. In another embodiment, the fatty alcohol composition comprises 30 to 40 wt % of one or more fatty alcohols. In another embodiment, the fatty alcohol composition comprises 40 to 50 wt % of one or more fatty alcohols. In another embodiment, the fatty alcohol composition comprises 50 to 60 wt % of one or more fatty alcohols. In another embodiment, the fatty alcohol composition comprises 60 to 70 wt % of one or more fatty alcohols. In another embodiment, the fatty alcohol composition comprises 70 to 80 wt % of one or more fatty alcohols. In another embodiment, the fatty alcohol composition comprises 80 to 90 wt % of one or more fatty alcohols. In another embodiment, the fatty alcohol composition comprises 90 to 100 wt % of one or more fatty alcohols. In a preferred embodiment of the disclosure, the fatty alcohol composition comprises in the range of 50 to 100% of one or more fatty alcohol. In an even more preferred embodiment, the fatty alcohol composition comprises in the range of 60 to 100% of one or more fatty alcohols.

In one embodiment of the disclosure, the fatty alcohol composition comprises at least 30 wt % of one or more fatty alcohols. In another embodiment, the fatty alcohol composition comprises at least 35 wt % of one or more fatty alcohols. In another embodiment, the fatty alcohol composition comprises at least 40 wt % of one or more fatty alcohols. In another embodiment, the fatty alcohol composition comprises at least 45 wt % of one or more fatty alcohols. In another embodiment, the fatty alcohol composition comprises at least 50 wt % of one or more fatty alcohols. In another embodiment, the fatty alcohol composition comprises at least 55 wt % of one or more fatty alcohols. In another embodiment, the fatty alcohol composition comprises at least 60 wt % of one or more fatty alcohols.

In a preferred embodiment of the disclosure, the fatty alcohol composition is substantially dry, i.e. it contains at most only a small amount of water. In a preferred embodiment of the disclosure, the fatty alcohol composition does not contain any compound that would interfere deleteriously with the oxidation. Compounds that are considered deleterious to the reaction conditions are for example: carboxylic acids, amino acids, amines, 1,2-diols, 1,3-diols, sulfides, and other chelating compounds.

The presently disclosed methods are contemplated to be especially useful for oxidation of alcohols originating from feedstocks or pheromone alcohols. In one embodiment of the present disclosure, the fatty alcohol composition originates from a feedstock. In one embodiment of the disclosure, the fatty alcohol composition comprises pheromone alcohols.

In one embodiment, the fatty alcohol composition comprises one or more of the fatty alcohols disclosed herein, wherein each of the one or more fatty alcohols are present in an amount of 0.1 to 100 wt %. In a specific embodiment of the present disclosure, the fatty alcohol composition comprises (Z)-11-hexadecen-1-ol. In a further specific embodiment of the present disclosure, the fatty alcohol composition comprises 10 to 100 wt % (Z)-11-hexadecen-1-ol. In a further specific embodiment of the present disclosure, the fatty alcohol composition comprises 50 to 100 wt % (Z)-11-hexadecen-1-ol. In a specific embodiment of the present disclosure, the fatty alcohol composition comprises (Z)-9-hexadecen-1-ol. In a further specific embodiment of the present disclosure, the fatty alcohol composition comprises 1 to 10 wt % (Z)-9-hexadecen-1-ol. In a specific embodiment of the present disclosure, the fatty alcohol composition comprises hexadecan-1-ol. In a further specific embodiment of the present disclosure, the fatty alcohol composition comprises 1 to 15 wt % hexadecan-1-ol. In a specific embodiment of the present disclosure, the alcohol composition comprises 50 to 98 wt % (Z)-11-hexadecen-1-ol, 1 to 10 wt % (Z)-9-hexadecen-1-ol, and 1 to 15% hexadecan-1-ol.

In one embodiment of the present disclosure, the fatty alcohol composition comprises 10 to 100 wt % (Z)-11-Hexadecen-1-ol. In one embodiment of the present disclosure, the fatty alcohol composition comprises 1 to 10 wt % (Z)-9-hexadecen-1-ol. In one embodiment of the disclosure, the fatty alcohol composition comprises 1 to 15 wt % hexadecan-1-ol. In one embodiment of the present disclosure, the fatty alcohol composition comprises 1 to 20 wt % monounsaturated pentadecen-1-ol. In a specific embodiment, the fatty alcohol composition comprises 10 to 100 wt % (Z)-11-Hexadecen-1-ol, 1 to 10 wt % (Z)-9-hexadecen-1-ol, 1 to 15 wt % hexadecan-1-ol, and 1 to 20 wt % monounsaturated pentadecen-1-ol.

### Fatty Aldehydes

The present disclosure relates to fatty aldehyde compositions comprising at least one fatty aldehyde. In one embodiment of the disclosure, the fatty aldehyde composition consists of or comprises a single fatty aldehyde. In another embodiment, the fatty aldehyde composition consist of or comprise a mixture of a few fatty aldehydes, such as 2 to 5 fatty aldehydes. In yet another embodiment, the fatty aldehyde composition consists of or comprises several fatty aldehydes, such as 6 or more fatty aldehydes.

The fatty aldehyde may be a saturated fatty aldehyde, a desaturated fatty aldehyde. In one embodiment of the disclosure, the fatty aldehyde composition comprises solely saturated fatty aldehydes. In another embodiment, the fatty aldehyde composition comprises solely desaturated fatty aldehydes. In yet another embodiment of the present disclosure, the aldehyde composition comprises both saturated and desaturated fatty aldehyde.

In one embodiment, the fatty aldehyde has a chain length of 8. In another embodiment, the fatty aldehyde has a chain length of 9. In another embodiment, the fatty aldehyde has a chain length of 10. In another embodiment, the fatty aldehyde has a chain length of 11. In another embodiment, the fatty aldehyde has a chain length of 12. In another embodiment, the fatty aldehyde has a chain length of 13. In another embodiment, the fatty aldehyde has a chain length of 14. In another embodiment, the fatty aldehyde has a chain length of 15. In another embodiment, the fatty aldehyde has a chain length of 16. In another embodiment, the fatty aldehyde has a chain length of 17. In another embodiment, the fatty aldehyde has a chain length of 18. In another embodiment, the fatty aldehyde has a chain length of 19. In another embodiment, the fatty aldehyde has a chain length of 20. In another embodiment, the fatty aldehyde has a chain length of 21. In another embodiment, the fatty aldehyde has a chain length of 22.

Fatty aldehydes may be branched or unbranched (i.e. linear or “straight-chain”). In a preferred embodiment of the present disclosure, the fatty aldehyde is unbranched.

In a preferred embodiment of the disclosure, the fatty aldehyde has a chain length of 12 to 16. In a further embodiment of the disclosure, the fatty aldehyde is unbranched and has a chain length of 12 to 16. In an even more preferred embodiment of the disclosure, the fatty aldehyde is unbranched and has a chain length of 12. In another even more preferred embodiment, the fatty aldehyde is unbranched and has a chain length of 14. In another even more preferred embodiment, the fatty aldehyde is unbranched and has a chain length of 16.

In one embodiment of the present disclosure, the fatty aldehyde is a saturated fatty aldehyde. In one embodiment of the disclosure, the fatty aldehyde is a saturated fatty aldehyde having a carbon chain length of 8, 9, 10, 11, 12, 13, 14, 15, 16, 17, 18, 19, 20, 21, or 22.

In one embodiment of the present disclosure, the fatty aldehyde is a desaturated fatty aldehyde. The double bond of the desaturated fatty aldehyde may have either E or Z configuration, except if the double bond is a terminal double bond. In one embodiment of the disclosure, the fatty aldehyde comprises one or more E configured double bonds. In one embodiment of the disclosure, the fatty aldehyde comprises one or more Z configured double bonds. In yet another embodiment, the fatty aldehyde comprises one or more E configured double bonds and one or more Z configured double bonds.

In some embodiments, the fatty aldehyde is a desaturated fatty aldehyde. The desaturated fatty aldehydes may be:
(Z)-Δ3 desaturated fatty aldehydes having a carbon chain length of 8, 9, 10, 11, 12, 13, 14, 15, 16, 17, 18, 19, 20, 21 or 22;
(E)-Δ3 desaturated fatty aldehydes having a carbon chain length of 8, 9, 10, 11, 12, 13, 14, 15, 16, 17, 18, 19, 20, 21 or 22;
(Z)-Δ5 desaturated fatty aldehydes having a carbon chain length of 8, 9, 10, 11, 12, 13, 14, 15, 16, 17, 18, 19, 20, 21 or 22;
(E)-Δ5 desaturated fatty aldehydes having a carbon chain length of 8, 9, 10, 11, 12, 13, 14, 15, 16, 17, 18, 19, 20, 21 or 22;
(Z)-Δ6 desaturated fatty aldehydes having a carbon chain length of 8, 9, 10, 11, 12, 13, 14, 15, 16, 17, 18, 19, 20, 21 or 22;
(E)-Δ6 desaturated fatty aldehydes having a carbon chain length of 8, 9, 10, 11, 12, 13, 14, 15, 16, 17, 18, 19, 20, 21 or 22;
(Z)-Δ7 desaturated fatty aldehydes having a carbon chain length of 8, 9, 10, 11, 12, 13, 14, 15, 16, 17, 18, 19, 20, 21 or 22;
(E)-Δ7 desaturated fatty aldehydes having a carbon chain length of 8, 9, 10, 11, 12, 13, 14, 15, 16, 17, 18, 19, 20, 21 or 22;
(Z)-Δ8 desaturated fatty aldehydes having a carbon chain length of 9, 10, 11, 12, 13, 14, 15, 16, 17, 18, 19, 20, 21 or 22;
(E)-Δ8 desaturated fatty aldehydes having a carbon chain length of 9, 10, 11, 12, 13, 14, 15, 16, 17, 18, 19, 20, 21 or 22;
(Z)-Δ9 desaturated fatty aldehydes having a carbon chain length of 10, 11, 12, 13, 14, 15, 16, 17, 18, 19, 20, 21 or 22;
(E)-Δ9 desaturated fatty aldehydes having a carbon chain length of 10, 11, 12, 13, 14, 15, 16, 17, 18, 19, 20, 21 or 22;
(Z)-Δ10 desaturated fatty aldehydes having a carbon chain length of 11, 12, 13, 14, 15, 16, 17, 18, 19, 20, 21 or 22;
(E)-Δ10 desaturated fatty aldehydes having a carbon chain length of 11, 12, 13, 14, 15, 16, 17, 18, 19, 20, 21 or 22;
(Z)-Δ11 desaturated fatty aldehydes having a carbon chain length of 12, 13, 14, 15, 16, 17, 18, 19, 20, 21 or 22;
(E)-Δ11 desaturated fatty aldehydes having a carbon chain length of 12, 13, 14, 15, 16, 17, 18, 19, 20, 21 or 22;
(Z)-Δ12 desaturated fatty aldehydes having a carbon chain length of 13, 14, 15, 16, 17, 18, 19, 20, 21 or 22;
(E)-Δ12 desaturated fatty aldehydes having a carbon chain length of 13, 14, 15, 16, 17, 18, 19, 20, 21 or 22;
(Z)-Δ13 desaturated fatty aldehydes having a carbon chain length of 14, 15, 16, 17, 18, 19, 20, 21 or 22; and
(E)-Δ13 desaturated fatty aldehydes having a carbon chain length of 14, 15, 16, 17, 18, 19, 20, 21 or 22.

In some embodiments, the fatty aldehydes are desaturated fatty aldehydes having a carbon chain length of 12, such as:
(Z)-Δ5 desaturated fatty aldehydes having a carbon chain length of 12;
(E)-Δ5 desaturated fatty aldehydes having a carbon chain length of 12;
(Z)-Δ6 desaturated fatty aldehydes having a carbon chain length of 12;
(E)-Δ6 desaturated fatty aldehydes having a carbon chain length of 12;
(Z)-Δ7 desaturated fatty aldehydes having a carbon chain length of 12;
(E)-Δ7 desaturated fatty aldehydes having a carbon chain length of 12;
(Z)-Δ8 desaturated fatty aldehydes having a carbon chain length of 12;
(E)-Δ8 desaturated fatty aldehydes having a carbon chain length of 12;
(Z)-Δ9 desaturated fatty aldehydes having a carbon chain length of 12;
(E)-Δ9 desaturated fatty aldehydes having a carbon chain length of 12;
(Z)-Δ10 desaturated fatty aldehydes having a carbon chain length of 12;
(E)-Δ10 desaturated fatty aldehydes having a carbon chain length of 12;
(Z)-Δ11 desaturated fatty aldehydes having a carbon chain length of 12; and
(E)-Δ11 desaturated fatty aldehydes having a carbon chain length of 12.

In some embodiments, the fatty aldehydes are desaturated fatty aldehydes having a carbon chain length of 14, such as:
(Z)-Δ5 desaturated fatty aldehydes having a carbon chain length of 14;
(E)-Δ5 desaturated fatty aldehydes having a carbon chain length of 14;
(Z)-Δ6 desaturated fatty aldehydes having a carbon chain length of 14;
(E)-Δ6 desaturated fatty aldehydes having a carbon chain length of 14;
(Z)-Δ7 desaturated fatty aldehydes having a carbon chain length of 14;
(E)-Δ7 desaturated fatty aldehydes having a carbon chain length of 14;
(Z)-Δ8 desaturated fatty aldehydes having a carbon chain length of 14;
(E)-Δ8 desaturated fatty aldehydes having a carbon chain length of 14;
(Z)-Δ9 desaturated fatty aldehydes having a carbon chain length of 14;
(E)-Δ9 desaturated fatty aldehydes having a carbon chain length of 14;
(Z)-Δ10 desaturated fatty aldehydes having a carbon chain length of 14;
(E)-Δ10 desaturated fatty aldehydes having a carbon chain length of 14;
(Z)-Δ11 desaturated fatty aldehydes having a carbon chain length of 14;
(E)-Δ11 desaturated fatty aldehydes having a carbon chain length of 14;
(Z)-Δ12 desaturated fatty aldehydes having a carbon chain length of 14;
(E)-Δ12 desaturated fatty aldehydes having a carbon chain length of 14;
(Z)-Δ13 desaturated fatty aldehydes having a carbon chain length of 14; and
(E)-Δ13 desaturated fatty aldehydes having a carbon chain length of 14.

In some embodiments, the fatty aldehydes are desaturated fatty aldehydes having a carbon chain length of 16, such as:
(Z)-Δ5 desaturated fatty aldehydes having a carbon chain length of 16;
(E)-Δ5 desaturated fatty aldehydes having a carbon chain length of 16;
(Z)-Δ6 desaturated fatty aldehydes having a carbon chain length of 16;
(E)-Δ6 desaturated fatty aldehydes having a carbon chain length of 16;
(Z)-Δ7 desaturated fatty aldehydes having a carbon chain length of 16;
(E)-Δ7 desaturated fatty aldehydes having a carbon chain length of 16;
(Z)-Δ8 desaturated fatty aldehydes having a carbon chain length of 16;
(E)-Δ8 desaturated fatty aldehydes having a carbon chain length of 16;
(Z)-Δ9 desaturated fatty aldehydes having a carbon chain length of 16;
(E)-Δ9 desaturated fatty aldehydes having a carbon chain length of 16;
(Z)-Δ10 desaturated fatty aldehydes having a carbon chain length of 16;
(E)-Δ10 desaturated fatty aldehydes having a carbon chain length of 16;
(Z)-Δ11 desaturated fatty aldehydes having a carbon chain length of 16;
(E)-Δ11 desaturated fatty aldehydes having a carbon chain length of 16;
(Z)-Δ12 desaturated fatty aldehydes having a carbon chain length of 16;
(E)-Δ12 desaturated fatty aldehydes having a carbon chain length of 16;
(Z)-Δ13 desaturated fatty aldehydes having a carbon chain length of 16; and
(E)-Δ13 desaturated fatty aldehydes having a carbon chain length of 16.

For example, the fatty aldehyde is an (E)7,(Z)9 desaturated fatty aldehyde having a carbon chain length of 10, 11, 12, 13, 14, 15, 16, 17, 18, 19, 20, 21 or 22. In some embodiments, the fatty aldehyde is an (E)3, (Z)8, (Z)11 desaturated fatty aldehyde having a carbon chain length of 12, 13, 14, 15, 16, 17, 18, 19, 20, 21 or 22, for example 14. In some embodiments, the fatty aldehyde is a (Z)9, (E)11, (E)13 desaturated fatty aldehyde having a carbon chain length of 14, 15, 16, 17, 18, 19, 20, 21 or 22. In some embodiments, the fatty aldehyde is a (Z)11,(Z)13 desaturated fatty aldehyde having a carbon chain length of 14, 15, 16, 17, 18, 19, 20, 21 or 22. In some embodiments, the fatty aldehyde is a (Z)9,(E)12 desaturated fatty aldehyde having a carbon chain length of 13, 14, 15, 16, 17, 18, 19, 20, 21 or 22. In some embodiments, the fatty aldehyde is a (E)7,(E)9 desaturated fatty aldehyde having a carbon chain length of 10, 11, 12, 13, 14, 15, 16, 17, 18, 19, 20, 21 or 22. In some embodiments, the fatty aldehyde is a (E)8,(E)10 desaturated fatty aldehyde having a carbon chain length of 11, 12, 13, 14, 15, 16, 17, 18, 19, 20, 21, or 22.

In other embodiments, the fatty aldehyde is an (E)7,(Z)9 desaturated fatty aldehyde having a carbon chain length of 14. In other embodiments, the desaturated fatty aldehyde is an (E)3, (Z)8, (Z)11 desaturated fatty aldehyde having a carbon chain length of 14. In other embodiments, the desaturated fatty aldehyde is a (Z)9, (E)11, (E)13 desaturated fatty aldehyde having a carbon chain length of 14. For example, the fatty aldehyde is an (E)7,(Z)9 desaturated fatty aldehyde having a carbon chain length of 12. In other embodiments, the desaturated fatty aldehyde is an (E)3, (Z)8, (Z)11 desaturated fatty aldehyde having a carbon chain length of 12. In other embodiments, the desaturated fatty aldehyde is a (Z)9, (E)11, (E)13 desaturated fatty aldehyde having a carbon chain length of 12. In other embodiments, the desaturated fatty aldehyde is a (E)8, (E)10 desaturated fatty aldehyde having a carbon chain length of 12. In other embodiments, the desaturated fatty aldehyde is a (E)7,(E)9 desaturated fatty aldehyde having a carbon chain length of 11. In other embodiments, the desaturated fatty aldehyde is a (Z)11,(Z)13 desaturated fatty aldehyde having a carbon chain length of 16. In other embodiments, the desaturated fatty aldehyde is a (Z)9,(E)12 desaturated fatty aldehyde having a carbon chain length of 14

In some embodiments the fatty aldehyde is (Z9, E12)-tetradecadien-1-al. Microbial cell factories and methods for obtaining the corresponding alcohol (Z9, E12)-tetradecadien-1-ol from a yeast cell are described in detail in application EP21183447.8 entitled “Methods and yeast cells for production of desaturated compounds” filed on 2 Jul. 2021 by same applicant. This alcohol can be converted to (Z9, E12)-tetradecadien-1-al using the method disclosed herein.

In some embodiments the fatty aldehyde is (Z11, Z13)-hexadecadien-1-al. Microbial cell factories and methods for obtaining the corresponding alcohol (Z11, Z13)-hexadecadien-1-ol from a yeast cell are described in detail in application EP21183459.3 entitled “Methods and yeast cells for production of desaturated compounds” filed on 2 Jul. 2021 by same applicant. This alcohol can be converted to Z11, Z13)-hexadecadien-1-al using the method disclosed herein.

In some embodiments the fatty aldehyde is (E8,E10)-dodecadien-1-al. Microbial cell factories and methods for obtaining the corresponding alcohol (E8,E10)-hexadecadien-1-ol from a yeast cell are described in detail in application WO 2021/123128. This alcohol can be converted to (E8,E10)-dodecadien-1-al using the method disclosed herein.

In some embodiments the fatty aldehyde is (Z11)-hexadecen-1-al. Microbial cell factories and methods for obtaining the corresponding alcohol (Z11)-hexadecen-1-ol from a yeast cell are described in detail in application WO 2016/207339. This alcohol can be converted to (Z11)-hexadecen-1-al using the method disclosed herein.

In a preferred embodiment of the disclosure, the fatty aldehyde has a double bond at position 9, 11, or 13, or double bonds at positions 9 and 11 or at positions 11 and 13; or the fatty aldehyde has a double bond at position 9 or 12, or double bonds at positions 9 and 12. In an even more preferred embodiment of the disclosure, the fatty aldehyde has a chain length of 12 and a double bond at position 9 or 11, or double bonds at positions 9 and 11; or the fatty aldehyde has a chain length of 14 and a double bond at position 9 or 12, or double bonds at positions 9 and 12; or the fatty aldehyde has a chain length of 14 and a double bond at position 9, 11, or 13, or double bounds at positions 9 and 11, or at positions 11 and 13. In another more preferred embodiment of the disclosure, the fatty aldehyde has a chain length of 14 and a double bond at position 9 or 11, or double bonds at positions 9 and 11. In another more preferred embodiment of the disclosure, the fatty aldehyde has a chain length of 16 and a double bond at position 9 or 11, or double bonds at positions 9 and 11. In other embodiments, the fatty aldehyde has a chain length of 16 and a double bond at position 11 or 13, or double bonds at positions 11 and 13. In other embodiments, the fatty aldehyde has a chain length of 12 and a double bond at position 8 or 10, or double bonds at positions 8 and 10.

In a specific embodiment, the fatty aldehyde is selected from the group consisting of tetradecan-1-al, pentadecan-1-al, hexadecan-1-al, pentadecen-1-al, (Z)-9-hexadecen-1-al, (Z)-11-hexadecen-1-al, (7E,9E)-undeca-7,9-dien-1-al, (11Z, 13Z)-hexadecadien-1-al, (9Z,12E)-tetradecadien-1-al, and (8E,10E)-dodecadien-1-al.

In a particular embodiment, the fatty aldehyde is (Z)-11-hexadecenal or (Z)-9-tetradecenal.

The fatty aldehyde composition may consist entirely of fatty aldehydes, or it may comprise fatty aldehydes and other compounds. In one embodiment of the disclosure, the fatty aldehyde composition comprises 5 to 10 wt % of one or more fatty aldehydes. In another embodiment, the fatty aldehyde composition comprises 10 to 20 wt % of one or more fatty aldehydes. In another embodiment, the fatty aldehyde composition comprises 20 to 30 wt % of one or more fatty aldehydes. In another embodiment, the fatty aldehyde composition comprises 30 to 40 wt % of one or more fatty aldehydes. In another embodiment, the fatty aldehyde composition comprises 40 to 50 wt % of one or more fatty aldehydes. In another embodiment, the fatty aldehyde composition comprises 50 to 60 wt % of one or more fatty aldehydes. In another embodiment, the fatty aldehyde composition comprises 60 to 70 wt % of one or more fatty aldehydes. In another embodiment, the fatty aldehyde composition comprises 70 to 80 wt % of one or more fatty aldehydes. In another embodiment, the fatty aldehyde composition comprises 80 to 90 wt % of one or more fatty aldehydes. In another embodiment, the fatty aldehyde composition comprises 90 to 100 wt % of one or more fatty aldehydes. In a preferred embodiment of the disclosure, the fatty aldehyde composition comprises in the range of 50 to 100% of one or more fatty aldehyde. In an even more preferred embodiment, the fatty aldehyde composition comprises in the range of 60 to 100% of one or more fatty aldehydes.

In one embodiment of the disclosure, the fatty aldehyde composition comprises at least 30 wt % of one or more fatty aldehydes. In another embodiment, the fatty aldehyde composition comprises at least 35 wt % of one or more fatty aldehydes. In another embodiment, the fatty aldehyde composition comprises at least 40 wt % of one or more fatty aldehydes. In another embodiment, the fatty aldehyde composition comprises at least 45 wt % of one or more fatty aldehydes. In another embodiment, the fatty aldehyde composition comprises at least 50 wt % of one or more fatty aldehydes. In another embodiment, the fatty aldehyde composition comprises at least 55 wt % of one or more fatty aldehydes. In another embodiment, the fatty aldehyde composition comprises at least 60 wt % of one or more fatty aldehydes. In a preferred embodiment, the fatty aldehyde composition comprises at least 70 wt % of one or more fatty aldehydes. In another preferred embodiment, the fatty aldehyde composition comprises at least 80 wt % of one or more fatty aldehydes. In another preferred embodiment, the fatty aldehyde composition comprises at least 90 wt % of one or more fatty aldehydes.

The embodiments of the aldehyde composition as outlined herein preferably refers to the isolated, purified aldehyde composition.

### Catalyst Composition

The present disclosure regards oxidation of primary alcohols to produce the corresponding aldehydes. The oxidation of primary alcohol to aldehyde is catalysed by a catalyst composition.

The catalyst composition comprises a copper (I) source, such as for example a copper (I) salt. The copper (I) source is a substance or mixtures of substances containing a copper (I) compound available for the desired catalysis involving copper (I). Examples include, among others, copper (I) chloride, copper (I) bromide, copper (I) iodide, copper (I) cyanide, copper (I) oxide, copper (I) trifluoromethanesulfonate, tetrakis (acetonitrile) copper (I) tetrafluoroborate, tetrakis (acetonitrile) copper (I) tetraphenylborate, tetrakis (acetonitrile) copper (I) hexafluorophosphate, tetrakis (acetonitrile) copper (I) trifluoromethanesulfonate, copper (I) sulfide, copper (I) thiocyanate, Cu[1,3-bis(2,6-diisopropylphenyl) imidazol-2-ylidene]Cl, Cu[1,3-bis(2,6-diisopropylphenyl) imidazol-2-ylidene]Br, CuBr(1,10-phenanthroline)₂, CuCl(1,10-phenanthroline)]₂, CuI(1,10-phenanthroline)₂, copper (I) trifluoroacetate, [Cu(PPh₃)₃]Br, [Cu(PPh₃)₃]F, [Cu(PPh₃)₃]Cl, Cu(OCOR²), Cu(SR²), Cu(SR²₂) Br, Cu(SR²₂)Cl, Cu(SR²₂)I, Cu(OSO₂R²), CuOR², wherein R² is selected from alkyl, preferably C₁-C₂₀ alkyl, optionally substituted with one or more aryl, alkoxy and aryloxy and from aryl, preferably C₅-C₇ aryl, optionally substituted with one or more alkyl, aryl, alkoxy and aryloxy and mixtures thereof. In addition, the copper (I) source can be a substance or mixtures of substances containing copper in any other oxidation state, provided it can be converted to copper in the oxidation state of +1 by means of reduction or oxidation, either chemically or electrochemically.

In a preferred embodiment of the present disclosure, the copper (I) source comprises copper present in oxidation state of +1.

In one embodiment of the disclosure, the copper (I) source is soluble in an organic solvent. In a preferred embodiment, the organic solvent is acetonitrile. Solubility of reagents generally improves reaction rate. In a preferred embodiment of the disclosure, the copper (I) source is a copper (I) salt comprises a counter ion, i.e. a negatively charged ion, which has good solubility in the organic solvent. Examples of negatively charged ions which are generally considered to have good solubility in organic solvents such as acetonitrile includes triflate, tetrafluoroborate, hexafluorophosphate, and halides.

The copper (I) source maybe further comprise ligands coordinated to copper. Examples copper one sources having coordinated ligands include tetrakisacetonitrile copper (I) triflate, tetrakisacetonitrile copper (I) tetrafluoroborate, tetrakisacetonitrile copper (I) hexafluorophosphate, tetrakisacetonitrile copper (I) halide, CuBr(1,10-phenanthroline)₂, CuCl(1,10-phenanthroline)]₂, and CuI(1,10-phenanthroline)₂.

In a preferred embodiment of the present disclosure, the copper (I) source is selected from the group consisting of tetrakisacetonitrile copper (I) triflate, tetrakisacetonitrile copper (I) tetrafluoroborate, tetrakisacetonitrile copper (I) hexafluorophosphate, and tetrakisacetonitrile copper (I) halide.

Copper (I) ions can be generated in situ from a copper (II) compound and a reductant. Thus, in one embodiment, the copper (l) source comprises a copper (II) compound and a reductant. In one embodiment, the copper (I) source is a copper (II) compound and a reductant.

In one embodiment, the copper (II) compound is a copper (II) salt. In one embodiment, the copper (II) salt comprises counter ion that is soluble in organic solvents. Counter ions that are soluble in organic solvents typically comprise larger organic moieties and/or delocalisable (e.g. by resonance or induction) negative charge. In one embodiment, the copper (II) salt is selected from the group consisting of copper (II) triflate, copper (II) tetrafluoroborate, copper (II) hexafluorophosphate, copper (II) bromide, copper (II) chloride, copper (II) iodide, and copper (II) perchlorate.

The reductant as disclosed herein is capable of reducing copper (II) to copper (I). The reductant can be either of an organic or an inorganic reductant. In one embodiment of the present disclosure, the reductant is selected from the group consisting of copper metal, zinc metal, aluminium metal, sodium hydrogensulfite, formic acid, salts of formic acid, oxalic acid, and salts of oxalic acid. The metal-based reductants may advantageously be on powder, pellet, shavings, or otherwise finely divided form. The reductant may advantageously be chosen as to not produce any side product(s), or to produce side product(s) that are easily removed, for instance by evaporation. In one embodiment of the present disclosure, the copper (I) source comprises a copper (II) salt and copper metal.

In one embodiment of the present disclosure, the catalyst composition of the present disclosure comprises a ligand. It is contemplated that the role of the ligand is to coordinate to the copper (I) of the catalyst composition, thereby improving the solubility of the copper (I), stabilising the catalyst composition, and/or improving the catalytic activity of the catalyst composition.

Suitable ligands include ligands coordinating via nitrogen, oxygen, phosphorous, or other atoms having a lone-pair. In one embodiment of the present disclosure, the ligand coordinates via a moiety selected from the group consisting of a pyridine, a triarylphosphine, a diarylphosphine, an amine, an imidazole, a pyrazole, a pyrrole, a triazole, a tetrazole, an imine, an enamine, a phenol, or a moiety comprising any one of the listed moieties. In a preferred embodiment, the ligand coordinates via a pyridine moiety.

The ligand may be a monodentate or a polydentate ligand. In one embodiment of the present disclosure, the ligand is a monodentate ligand. In another embodiment of the disclosure, the ligand is a bidentate ligand. In another embodiment, the ligand is a polydentate ligand coordinating with 3 or more atoms.

In one embodiment of the present disclosure, the catalyst composition comprises a single type of ligand as described herein. In another embodiment of the present disclosure, the catalyst composition comprises a mixture of two or more types of ligands as described herein.

In one embodiment of the disclosure, the ligand is selected from the group consisting of DETA, PMDETA, TETA, HMTETA, Me₆TREN, cyclam, Me₆cyclam, DMCBCy, bpy, dNbpy, 1,10-Phen, tpy, tNtpy, BPMPrA, BPMOA, BPMODA, TPMA, and TPEA. In one embodiment of the disclosure, the ligand is a secondary amine, such as a secondary amine having bulky substituents (i.e. to reduce nucleophilicity of the amine). In one embodiment of the present disclosure, the ligand is a bidentate nitrogen ligand. In one embodiment, the ligand comprises a 2,2′-bipyridine moiety or a 2,2′-bipyrimidine moiety. In one embodiment, the ligand is selected from the group consisting of 4,4′-dimethyl-2,2′-bipyridine, 5,5′-dimethyl-2,2′-bipyridine 2,2′-bipyrimidine, 2,2′-bipyridine-4,4′-dicarboxylic acid or an ester thereof, 2,2′-bipyridine-5,5′-dicarboxylic acid or an ester thereof. In a preferred embodiment of the present disclosure, the ligand is 2,2′-bipyridine (bpy).

The catalyst composition of the present disclosure preferably comprises an aminoxyl radical compound, i.e. a compound having a N—O′ functional group. In a further embodiment of the present disclosure, the aminoxyl radical compound is a dialkyl aminoxyl radical compound. In a further embodiment of the present disclosure, the aminoxyl radial compound is piperidine N-oxide or a derivative thereof. In a further embodiment, the aminoxyl radical compound is a substituted piperidine N-oxide. In a further embodiment, the aminoxyl radical compound is (2,2,6,6-tetramethylpiperidin-1-yl)oxyl (TEMPO) or a derivative thereof. In one embodiment of the present disclosure, the aminoxyl radical compound is selected from the group consisting of TEMPO, (4-hydroxy-2,2,6,6-tetramethylpiperidin-1-yl)oxyl (4-OH-TEMPO), 4-acetamido-TEMPO, 4-hydroxy-TEMPO benzoate, 4-amino-TEMPO, 2-azaadamantane-N-oxyl, 9-azabicyclo[3.3.1]nonane N-oxyl, 4-carboxy-TEMPO, 4-maleimido-TEMPO, 4-methoxy-TEMPO, 1-methyl-2-azaadamantane-N-oxyl, 4-oxo-TEMPO, and a polymer functionalised with any of said aminoxyl radical compounds. In a preferred embodiment of the present disclosure, the aminoxyl radical compound is selected from the group consisting of TEMPO or (4-hydroxy-2,2,6,6-tetramethylpiperidin-1-yl)oxyl (4-OH-TEMPO). It is contemplated that the aminoxyl radical compound is part of the catalytic cycle which effect oxidation of the fatty alcohol composition of the present disclosure. It is acknowledged that TEMPO and its derivatives described herein act as catalysts for the oxidation of alcohol functional groups to aldehyde functional groups while the oxidant is O₂. However, as used herein, TEMPO and the derivatives disclosed herein are also termed “oxidants”.

In one embodiment of the present disclosure, the catalyst composition comprises a base. While some specific bases are mentioned herein below, it is contemplated many different bases will be useful in carrying out the present disclosure. In one embodiment of the present disclosure, the base is an organic base. Using an organic base may be advantageous as it may effect solubility of the base in the reaction medium as disclosed herein. In one embodiment of the present disclosure, the base is a nitrogen base. In one embodiment, the base is a Schiff base. In one embodiment, the base is an oxygen base. In one embodiment of the present disclosure, the base is selected from the group consisting of 1-methylimidazole, 1,8-diazabicyclo[5.4.0]undec-7-ene, 1,5-diazabicyclo[4.3.0]non-5-ene, 1,5,7-triazabicyclo[4.4.0]dec-5-ene, 1,1,3,3-tetramethylguanidine, 7-methyl-1,5,7-triazabicyclo[4.4.0]dec-5-ene, and potassium t-butoxide. In one embodiment of the present disclosure, the base is selected from the group consisting of: 1-methyl imidazole, potassium tert-butoxide, or 1,8-diazabicyclo(5.4.0) undec-7-ene (DBU).

In one embodiment of the disclosure, the elements of the catalyst composition as provided herein may be mixed to form the catalyst composition before the catalyst composition is added to the reaction mixture. In another embodiment, the elements of the catalyst composition may be added separately to the reaction mixture. In another embodiment, a subset of the elements of the catalyst composition may be mixed and added to reaction mixture, whereas the remaining elements of the catalyst composition is added separately and/or pre-mixed and then added to the reaction mixture.

### Method of Oxidation

The present disclosure provides for methods of converting a fatty alcohol composition to a fatty aldehyde composition. In particular, the conversion of the fatty alcohol composition to the fatty aldehyde composition is an oxidation of the fatty alcohol composition.

The presently disclosed methods are contemplated to be useful for the conversion of various different primary alcohol composition, e.g. composition comprising primary alcohols having chain lengths of at least two carbon atoms. The disclosed methods are nevertheless especially suitable for the oxidation of primary alcohols having chain lengths of eight or more carbon atoms (i.e. fatty alcohols), as further defined in the sections “fatty alcohols” and “fatty aldehydes” herein. Other known methods of carrying out oxidation will often only produce fatty aldehydes compositions in low yield and/or in low purity. “Over oxidation”, i.e. further oxidation of the aldehyde to the corresponding carboxylic acid is often a major contributor to the low reaction yields and/or purity of the aldehyde composition. Other known methods of carrying out oxidation of short primary alcohol may not be suitable for oxidation of fatty alcohols, because oxidation may be incomplete, “over oxidation” may occur, and/or it may be infeasible to purify the reaction product. In further relation to oxidation of fatty alcohols to fatty aldehydes, other known methods will often use a relatively large amount of solvent and/or the purification of the reaction product, as outlined in the background section. However, the presently disclosed methods make use of a relatively small volume of solvent for the oxidation reaction, as well as a relative small amount of solvent for the purification of the reaction product. The presently disclosed methods are also advantageous, as they are scalable, i.e. they work on both on small scale (e.g. less than 10 g fatty alcohol composition) or on a large scale (e.g. more than 100 g fatty alcohol composition, such as more than 500 g fatty alcohol composition). Other known methods of oxidising fatty alcohols to the corresponding fatty aldehydes might work well on a small scale (e.g. less than 10 g fatty alcohol composition), but they may not be scalable, i.e. they may not provide for good reaction yields or product purity at a larger scale (e.g. more than 100 g fatty alcohol composition, such as more than 500 g fatty alcohol composition). It is advantageous to obtain an aldehyde composition which is relatively free of by-products such as the corresponding fatty carboxylic acids or unreacted fatty alcohol, as this eliminates the need for time-consuming or expensive purification steps such as distillation. Each of the above mentioned features (small solvent volume, scalability, and substrate scope) makes the presently disclosed methods especially suitable for use in industry.

One embodiment of the present disclosure provides for a method of converting a fatty alcohol to a fatty aldehyde, said method comprising the steps of:
a. providing a reaction mixture comprising a fatty alcohol composition comprising the fatty alcohol, a catalyst composition, and a solvent, and
b. exposing the reaction mixture to at least 0.25 ml oxygen per minute per gram of fatty alcohol by means of bubbling a gas mixture comprising oxygen through the reaction mixture.
   thereby obtaining the fatty aldehyde.

In one embodiment of the present disclosure, a method is provided for large scale conversion of a fatty alcohol into a fatty aldehyde, said method comprising the steps of:
a) providing a reaction mixture comprising at least 1 kilogram of fatty alcohol, a catalyst comprising a copper source, at least 1 kilogram of solvent, and a water absorbing or adsorbing material absorbing or adsorbing water, and
b) dissolving at least 0.01 μmol O₂ per minute per μmol copper in the reaction mixture or at least 0.001 μmol O₂ per minute per μmol initial fatty alcohol in the reaction mixture to the reaction mixture by feeding a gas or a liquid comprising O₂ into the reaction medium and thereby oxidizing more than 50 wt % of the fatty alcohol into fatty aldehyde and less than 50 wt % into fatty acid.

The presently disclosed methods can be carried out without any external cooling and without any external heating. However, the present oxidation reactions are generally exothermic, and accordingly the reaction mixture is expected to rise in temperature over the course of the reaction. In one embodiment of the present disclosure, the reaction is carried out at 5 to 80° C., such as 10 to 70° C., such as 15 to 65° C. In a further embodiment of the reaction, the reaction mixture is exposed to oxygen at 5 to 80° C., such as to 70° C., such as 15 to 65° C.

The presently disclosed methods can be carried out at ambient pressure or at elevated pressure. In one embodiment, the exposure of the reaction mixture to oxygen is performed at a pressure of 0.5 to bar, such as 0.5 to 30 bar, such as 0.6 to 20 bar, such as 0.7 to 10 bar, such as 0.8 to 5 bar. In one embodiment, the exposition of the reaction mixture to oxygen is performed at a pressure of 0.5 to 0.8 bar, 0.8 to 1.2 bar, 1.2 to 1.5 bar, 1.5 to 2 bar, 2 to 5 bar, 5 to 10 bar, 10 to 20 bar, or 20 to 30 bar. In one embodiment of the present disclosure exposition of the reaction mixture to oxygen is performed at a pressure of 0.8 to 1.2 bar. However, it is contemplated that the presently disclosed methods can be carried out pressures lower than 0.5 bar or 0.8 bar, provided the amount of oxygen provided to the reaction mixture is as disclosed herein. In one embodiment, the pressure disclosed herein is the pressure in the reaction vessel wherein the exposure of the reaction mixture to oxygen is carried out. In one embodiment, the pressure disclosed herein is the partial pressure of oxygen in the reaction vessel.

In additional or alternative embodiments, the O₂ is added to the reaction medium by mixing the reaction mixture with a gas (such as air) or a liquid comprising O₂, optionally enriched with O₂. The said mixing can be made made by bubbling a gas mixture comprising O₂ through the reaction mixture.

In some embodiments, the copper source of the present disclosure comprises a copper (I) salt or a combination of copper (II) and a reductant.

### Oxygen Transfer Rate

It is an essential element of the present disclosure that the amount of oxygen supplied to the reaction mixture is above a certain threshold. The present inventors have found a surprising improvement to the reaction yield at high oxygen transfers rates.

In one embodiment of the present disclosure, the reaction mixture is exposed to at least 0.3 ml oxygen per minute per gram of fatty alcohol composition, such as at least 0.4 ml, 0.5 ml, 0.6 ml, 0.7 ml, 0.8 ml, 0.9 ml, 1.0 ml, 1.1 ml, 1.2 ml, 1.3 ml, 1.4 ml, such as at least 1.5 ml oxygen per minute per gram of fatty alcohol composition. In a preferred embodiment of the present disclosure, the reaction mixture is exposed to at least 1.5 ml oxygen per minute per gram of fatty alcohol composition.

In one embodiment of the present disclosure, the reaction mixture is exposed to at least 0.3 ml oxygen per minute per gram of fatty alcohol, such as at least 0.4 ml, 0.5 ml, 0.6 ml, 0.7 ml, 0.8 ml, 0.9 ml, 1.0 ml, 1.1 ml, 1.2 ml, 1.3 ml, 1.4 ml, such as at least 1.5 ml oxygen per minute per gram of fatty alcohol. In a preferred embodiment of the present disclosure, the reaction mixture is exposed to at least 1.5 ml oxygen per minute per gram of fatty alcohol.

As used herein, whenever a volume of gas is described, it is intended that this corresponds to the volume of the gas at essentially 1 bar of pressure.

In one embodiment of the present disclosure, the reaction mixture is exposed to at least 60 ml oxygen per minute per mol of fatty alcohol, such as at least 100 ml, 150 ml, 200 ml, 250 ml, 300 ml, 350 ml, 400 ml, such as at least 450 ml oxygen per minute per mol of fatty alcohol. In a preferred embodiment of the present disclosure, the reaction mixture is exposed to at least 450 ml oxygen per minute per mol of fatty alcohol.

In one embodiment of the present disclosure, the reaction mixture is exposed to at least 10 μmol oxygen per minute per gram of fatty alcohol, such as at least 12 μmol, 16 μmol, 20 μmol, 24 μmol, 28 μmol, 32 μmol, 36 μmol, 40 μmol, 44 μmol, 48 μmol, 52 μmol, 56 μmol, 60 μmol oxygen per minute per gram of fatty alcohol. In a preferred embodiment of the present disclosure, the reaction mixture is exposed to at least 60 μmol oxygen per minute per gram of fatty alcohol.

In one embodiment of the present disclosure, the reaction mixture is exposed to at least 2.5 mmol oxygen per minute per mol of fatty alcohol, such as at least 4 mmol, 6 mmol, 8 mmol, 10 mmol, 12 mmol, 14 mmol, 16 mmol, such as at least 18 mmol oxygen per minute per mol of fatty alcohol. In a preferred embodiment of the present disclosure, the reaction mixture is exposed to at least 18 mmol oxygen per minute per mol fatty alcohol.

The oxygen provided to the reaction mixture of the present disclosure may be provided either as pure oxygen or as a gas mixture comprising oxygen. In one embodiment of the present disclosure, the gas mixture comprises 5 to 100% oxygen. In a further embodiment of the present disclosure, the gas mixture comprises 15-25% oxygen. In one embodiment of the present disclosure, the gas mixture comprises at least 90% oxygen. In one embodiment of the present disclosure, the gas mixture is substantially pure oxygen. As outlined herein in the section “water removal”, it is beneficial if the amount of water present in the reaction mixture is minimized. Accordingly, in a preferred embodiment of the present disclosure, the gas mixture does not comprise H₂O.

It is contemplated that the sufficient exposure of oxygen to the reaction mixture is achieved in part using a sufficient supply of oxygen as outlined herein but also by ensuring a high contact surface between the supplied gas mixture and the liquid phase of the reaction mixture. A high contact surface is important for ensuring a sufficiently high exposure of oxygen to the reaction mixture, such as by ensuring a sufficiently high dissolution of oxygen in the liquid phase of the reaction mixture. This can be achieved by using equipment for bubbling gas through a liquid, such as for instance sparging equipment. It is contemplated that increasing the sparging of the gas mixture through the solution improves oxygen transfer rate. It is contemplated that increasing the partial pressure of the oxygen supplied to the reaction mixture improves the oxygen transfer rate. It is contemplated that stirring the reaction mixture improves the oxygen transfer rate. Accordingly, it is desirable that the reaction mixture of the present disclosure is stirred. In one embodiment of the present disclosure, the gas mixture comprising oxygen is bubbled through the reaction mixture. In a further embodiment of the disclosure, the bubbling of gas mixture through the reaction is carried out with sparging equipment. In one embodiment of the present disclosure, the reaction mixture is stirred while it is exposed to oxygen.

In one embodiment of the present disclosure, the reaction mixture is exposed to oxygen for at least 5 minutes, such as at least 10 minutes, such as at least 20 minutes, such as at least 30 minutes, such as at least 40 minutes, such as at least 50 minutes, such as at least 60 minutes, such as at least 70 minutes, 80 minutes, 90 minutes, such as at least 100 minutes. It is contemplated that the exposure to oxygen does not need to be maintained for a continuous time as specified herein, but can be interrupted. Accordingly, in one embodiment of the present disclosure, the reaction mixture is exposed to oxygen for an uninterrupted period of at least 60 minutes, such as at least 70 minutes, 80 minutes, 90 minutes, such as at least 100 minutes. In another embodiment of the present disclosure, the reaction mixture is exposed to oxygen for two or more periods of time, wherein the combined periods of time add to at least 60 minutes, such as at least 70 minutes, 80 minutes, 90 minutes, such as at least 100 minutes. In one embodiment of the present disclosure, the exposure to O₂ is carried out in a bubble column reactor or in a trickle bed reactor.

It is contemplated that longer reaction times can lead to lower conversion and/or lower yields of the disclosed aldehyde composition. It is contemplated this is due to for example over-oxidation of the aldehyde and/or introduction of water to the reaction mixture beyond the drying capacity of the means of drying. In one embodiment of the present disclosure, the reaction mixture is exposed to oxygen for at most 2000 minutes, such as at most 1900 minutes, 1800 minutes, 1700 minutes, 1600 minutes, 1500 minutes, 1400 minutes, 1300 minutes, 1200 minutes, 1100 minutes, 1000 minutes, 900 minutes, 800 minutes, 700 minutes, 600 minutes, 500 minutes, 400 minutes, 350 minutes, 325 minutes, 300 minutes, 275 minutes, such as at most 250 minutes.

It is important that the amount of oxygen added to the reaction medium is balanced to the amount of fatty alcohol in the reaction medium and/or to the amount and effectiveness of the catalyst. The feed of oxygen to the reaction medium for optimal formation of aldehyde may also be influenced by the amount of fatty acid in the reaction medium, of which formation of higher amounts of acid requiring increased oxygen feed. Accordingly, in additional or alternative embodiments, the method described herein comprises adding at least 0.010, such as at least 0.020, such as at least 0.030, such as at least 0.040, such as at least 0.049, such as at least 0.060, such as at least 0.070, such as at least 0.080, such as at least 0.090, such as at least 0.100 μmol dissolved O₂ per minute per μmol copper in the reaction mixture and/or at least 0.0010, such as at least 0.0020, such as at least 0.0025, such as at least 0.0030, such as at least 0.0050, such as at least 0.0075, such as at least 0.0100 μmol dissolved O₂ per minute per μmol initial fatty alcohol in the reaction mixture and/or at least 0.010, such as at least 0.015, such as at least 0.020, such as at least 0.025, such as at least 0.030, such as at least 0.050, such as at least 0.075, such as at least 0.100 μmol dissolved O₂ per minute per μmol fatty acid in the reaction mixture.

In some embodiments, the method of the present disclosure further comprises dissolving at least 0.049 μmol dissolved O₂ per minute per μmol copper in the reaction mixture.

In some embodiments, the method of the present disclosure further comprises dissolving at least 0.02 μmol dissolved O₂ per minute per μmol copper in the reaction mixture, such as at least 0.03 μmol, such as at least 0.04 μmol dissolved O₂ per minute per μmol copper in the reaction mixture.

In some embodiments, the method of the present disclosure further comprises dissolving from 0.01 to 1.00 μmol dissolved O₂ per minute per μmol copper in the reaction mixture, such as from 0.01 to 0.80 μmol, such as from 0.01 to 0.60 μmol, such as from 0.01 to 0.40 μmol, such as from 0.01 to 0.20 μmol, such as from 0.01 to 0.10 μmol dissolved O₂ per minute per μmol copper in the reaction mixture.

In some embodiments, the method of the present disclosure further comprises dissolving at least 0.0025 μmol dissolved O₂ per minute per μmol initial fatty alcohol in the reaction mixture.

In some embodiments, the method of the present disclosure further comprises dissolving at least 0.002 μmol dissolved O₂ per minute per μmol initial fatty alcohol in the reaction mixture, such as at least 0.003 μmol, such as at least 0.004 μmol dissolved O₂ per minute per μmol initial fatty alcohol in the reaction mixture.

In some embodiments, the method of the present disclosure further comprises dissolving from 0.001 to 1.00 μmol dissolved O₂ per minute per μmol initial fatty alcohol in the reaction mixture, such as from 0.001 to 0.80 μmol, such as from 0.001 to 0.60 μmol, such as from 0.001 to 0.40 μmol, such as from 0.001 to 0.20 μmol, such as from 0.001 to 0.10 μmol dissolved O₂ per minute per μmol initial fatty alcohol in the reaction mixture.

In some embodiments, the method of the present disclosure further comprises dissolving at least 0.025 μmol dissolved O₂ per minute per μmol fatty acid in the reaction mixture.

In some embodiments, the method of the present disclosure further comprises dissolving at least 0.01 μmol dissolved O₂ per minute per μmol fatty acid in the reaction mixture, such as at least 0.02 μmol, such as at least 0.03 μmol, such as at least 0.04 μmol dissolved O₂ per minute per μmol fatty acid in the reaction mixture.

In some embodiments, the method of the present disclosure further comprises dissolving at least 10 μmol O₂, such as at least 20 μmol O₂, at least 40 μmol O₂, or at least 60 μmol O₂ per minute per gram of fatty alcohol in the reaction mixture, thereby obtaining the fatty aldehyde, optionally wherein the fatty alcohol and the fatty aldehyde are desaturated.

In some embodiments the method of the present disclosure further comprises dissolving O₂ in the reaction medium at a rate sufficient for maintaining at least 80% O₂ saturation in the reaction medium during the oxidation reaction, such as at least 85% O₂ saturation, such as at least 90% O₂ saturation, such as at least 95% O₂ saturation, such as at least 100% O₂ saturation.

In some embodiments, the gas or a liquid comprising O₂ is air, optionally enriched with O₂.

In some embodiments, the method of the present disclosure is provided wherein the feeding of gas or a liquid comprising O₂ into the reaction medium is made by pumping or bubbling a gas or liquid mixture comprising O₂ through the reaction mixture.

### Reaction Conditions

The present disclosure achieves conversion of a fatty alcohol composition to a fatty aldehyde composition using a relatively small volume of solvent. In particular, the previously reported methods of converting fatty alcohols to fatty aldehydes as outlined herein utilises a relatively large volume of solvent for the reaction mixture. Large solvent volumes are often considered infeasible in large-scale production because of the costs of the solvent, the environmental footprint, and because handling large reaction volumes can be challenging. Accordingly, the presently disclosed methods of oxidation can advantageously be employed for large scale production of fatty aldehyde compositions due to the relatively small solvent volume required. By “relatively small solvent volume” is meant a volume as outlined herein.

In one embodiment of the present disclosure, the reaction mixture comprises a solvent. The solvent forming part of the reaction mixture may be either a substantially pure solvent or it may be a mixture of solvents. Accordingly, a reference to a solvent of the reaction mixture can in one embodiment also mean a solvent mixture comprising two or more solvents.

In one embodiment of the present disclosure, the solvent is selected from the group consisting of acetonitrile, dimethyl sulfoxide (DMSO), dimethyl formamide (DMF), alkanes such as pentane, hexane, and heptane, cycloalkanes, petroleum ether such as heavy or light petroleum ether, dioxane, diethyl ether, dichloromethane, tetrahydrofuran, ethyl acetate, acetone, nitromethane, propylene carbonate, and a solvent mixture comprising any one of said solvents.

In one embodiment, the solvent is an aprotic solvent. It is advantageous that the solvent is aprotic, as protons such as those originating from OH-groups or amines may interfere deleteriously with the components such as for example the catalyst composition, such as for example by inactivating the base. In one embodiment of the present disclosure, the solvent is selected from the list consisting of acetonitrile, dimethyl sulfoxide (DMSO), dimethyl formamide (DMF), alkanes such as pentane, hexane, and heptane, cycloalkanes, petroleum ether such as heavy or light petroleum ether, dioxane, diethyl ether, dichloromethane, tetrahydrofuran, ethyl acetate, acetone, nitromethane, propylene carbonate, and a solvent mixture comprising any one of said solvents. In a preferred embodiment, the solvent is selected from the list consisting of acetonitrile, DMSO, DMF, and a solvent mixture comprising any one of said solvents. In a further preferred embodiment, the solvent is or comprises acetonitrile. In another preferred embodiment of the disclosure, the solvent is acetonitrile.

In one embodiment, the solvent is a polar solvent. It is advantageous that the solvent is polar as this improves the solubility of at least some of the components of the reaction mixture and/or the components of the gas mixture. In one embodiment of the present disclosure, the solvent is selected from dichloromethane, tetrahydrofuran, ethyl acetate, acetone, dimethyl formamide (DMF), acetonitrile, dimethyl sulfoxide (DMSO), nitromethane, propylene carbonate, and a solvent mixture comprising any one of said solvents. In a preferred embodiment, the solvent is selected from the group consisting of acetonitrile, DMSO, DMF, or a solvent mixture comprising any one of said solvents. In an even further preferred embodiment, the solvent is acetonitrile or a solvent mixture comprising acetonitrile. In yet a further preferred embodiment, the solvent is acetonitrile.

In assessing the relative amount of solvent utilised for a chemical reaction, the amount of the solvent can be compared to the amount of the reagents or one of the reagents being converted in the chemical reaction, or the amount of the product or one of the products obtained in the chemical reaction.

The amount of solvent in the reaction mixture can be compared to the amount of fatty alcohol composition. In one embodiment of the present disclosure, the weight of solvent in the reaction mixture is 0 to 2000% the weight of the fatty alcohol composition, such as 100 to 2000%, such as 100 to 1500%, such as 100 to 1000%, such as 100 to 500%. The fatty alcohol composition may comprise other chemical compounds than fatty alcohol. For the assessment of amount of solvent, it is preferred that these other compounds are excluded when calculating the amount of solvent. Furthermore, the fatty alcohol composition may comprise one or more solvent, i.e. “fatty alcohol composition solvent”. In a preferred embodiment of the present disclosure, the fatty alcohol composition solvent is disregarded when assessing the amount of fatty alcohol composition. In one embodiment of the present disclosure, the weight of solvent corresponds to 100 to 2000% the weight of the fatty alcohol or fatty alcohols of the fatty alcohol composition, such as 100 to 1500%, such as 100 to 1000%, such as 100 to 500%.

The amount of solvent in the reaction mixture can be compared to the amount of fatty aldehyde composition obtained from said reaction mixture. In one embodiment of the present disclosure, the weight of solvent in the reaction mixture is 100 to 2000% the weight of the fatty aldehyde composition, such as 100 to 1500%, such as 100 to 1000%, such as 100 to 500%. The fatty aldehyde composition may comprise other chemical compounds than fatty aldehyde. For the assessment of amount of solvent, it is preferred that these other compounds are excluded when calculating the amount of solvent. Furthermore, the fatty aldehyde composition may comprise one or more solvent, i.e. “fatty aldehyde composition solvent”. In a preferred embodiment of the present disclosure, the fatty aldehyde composition solvent is disregarded when assessing the amount of fatty aldehyde composition. In one embodiment of the present disclosure, the weight of solvent corresponds to 100 to 2000% the weight of the fatty aldehyde or fatty aldehydes of the fatty aldehyde composition, such as 100 to 1500%, such as 100 to 1000%, such as 100 to 500%.

In some embodiments, the solvent is a non-halogenated solvent. In some embodiments, the solvent is selected from acetonitrile, dimethyl sulfoxide (DMSO), dimethyl formamide (DMF), pentane, hexane, heptane, cycloalkane, petroleum ether, dioxane, diethyl ether, tetrahydrofuran, ethyl acetate, acetone, nitromethane, propylene carbonate, or a combination thereof.

### Conversion

The presently disclosed method is effective in converting a fatty alcohol composition to a fatty aldehyde composition in high yields and/or with low formation of by-products. As used herein, conversion may be based on either amount of substance of the substrate and/or product, or amount by weight of the substrate.

In one embodiment of the present disclosure, the conversion of fatty alcohol is at least 80%, such as at least 82%, such as at least 84%, such as at least 86%, such as at least 88%, such as at least 90% as assessed by the amount of substance. In another embodiment of the present disclosure, the conversion of fatty alcohol is at least 80%, such as at least 82%, such as at least 84%, such as at least 86%, such as at least 88%, such as at least 90% as assessed by the weight of the fatty alcohol. In a preferred embodiment, the conversion of fatty alcohol to fatty aldehyde is at least 80%, such as at least 82%, such as at least 84%, such as at least 86%, such as at least 88%, such as at least 90% as assessed by the amount of substance. In a preferred embodiment, the conversion of fatty alcohol to fatty aldehyde is at least 80%, such as at least 82%, such as at least 84%, such as at least 86%, such as at least 88%, such as at least 90%, such as at least 92%, such as at least 94%, such as at least 96%, such as at least 98% as assessed by the weight of the fatty alcohol and the fatty aldehyde.

The conversion can specifically be calculated as the ratio of the amount of substance of aldehyde to the amount of substance of aldehyde and alcohol combined, i.e. n(aldehyde)/(n(aldehyde)+n(alcohol)), wherein n designates the amount of substance. In one embodiment of the present disclosure, n(aldehyde)/(n(aldehyde)+n(alcohol)) is at least 80%, such as at least 82%, such as at least 84%, such as at least 86%, such as at least 88%, such as at least 90% as assessed by the amount of substance. In a preferred embodiment, the conversion of fatty alcohol to fatty aldehyde is at least 80%, such as at least 82%, such as at least 84%, such as at least 86%, such as at least 88%, such as at least 90%, such as at least 92%, such as at least 94%, such as at least 96%, such as at least 98%.

The disclosed method is effective at converting fatty alcohol to fatty aldehyde with little formation of side-products, such as the corresponding fatty acid, i.e. with little “over oxidation”. For catalysed oxidation of alcohols to aldehydes, avoiding formation of carboxylic acids such as fatty acids is paramount, because it is contemplated that carboxylic acids can inactivate the catalyst composition. In one embodiment of the present disclosure, less than 10%, such as less than 8%, such as less than 6%, such as less than 5% of the fatty alcohol is converted to fatty acid. In another embodiment, less than 10%, such as less than 8%, such as less than 6%, such as less than 5% of the fatty aldehyde formed is converted to fatty acid. In one embodiment of the disclosure, the ratio of fatty acid to fatty aldehyde in the fatty aldehyde composition is less than 10:90, such as less than 8:92, such as less than 6:94, such as less than 5:05. “Ratio” as used here means molar ratio. In some aspects, the ratio of fatty acid produced to fatty aldehyde produced is less than 10:90.

It is contemplated that the disclosed methods are also useful for the conversion of other alcohol compositions to aldehyde composition. For example, the it contemplated the disclosed methods are useful for the conversion of C₂-C₇ alcohols, i.e. ethanol, propanol, butanol, pentanol, hexanol, and heptanol, to the corresponding C₂-C₇ aldehydes, i.e. ethanal, propanal, butanal, pentanal, hexanal, and heptanal. It is contemplated that both straight-chain and branched derivatives of these substrates can be converted using the disclosed methods, provided the substrate comprise a primary alcohol. By way of example, it is contemplated the disclosed methods are useful for the conversion n-butanol to n-butanal and for the conversion of iso-butanol to iso-butanal. It is contemplated that the disclosed methods are also useful for desaturated, i.e. unsaturated derivatives of the substrates above.

In some embodiments, the present disclosure provides a method wherein the conversion of fatty alcohol to fatty aldehyde is at least 60 wt %, such as at least 80 wt %, such as at least 85 wt %, such as at least 87 wt %, such as at least 90 wt %, such as at least wt 95 wt %, such as at least 99 wt %.

In some embodiments, the present disclosure provides a method wherein the conversion of fatty alcohol to fatty acid is less than 40 wt %, such as less than 30 wt %, such as less than 20 wt %, such as less than 15 wt %, such as less than 10 wt %, such as less than 5 wt %, such as less than 1 wt %.

### Water Removal

Water is present in trace amounts in many chemicals and solvents. Chemicals and solvents are often referred to as being “dry” if they contain no water, or only contain an insignificant amount of water. Water may also be produced during chemical reactions. It is contemplated that better reactions yields can be achieved if efforts are made to remove water from the reaction mixture. This is because water is contemplated to deteriorate the catalyst composition.

In one embodiment of the present disclosure, substantially dry solvents and reagents, including gas mixtures and gasses, are employed in the methods of the present invention.

Some reagents and/or solvents may be challenging to completely dry before they are being used in the presently disclosed methods. Accordingly, water may be removed from the reaction mixture while the method is carried out. In one embodiment of the present disclosure, water is removed from the reaction mixture. In one embodiment of the present disclosure, water is continuously removed from the reaction mixture throughout the exposure of oxygen to the reaction mixture. In one embodiment of the present disclosure, water is removed from the reaction mixture by adding a means of drying to the reaction mixture. In one embodiment of the disclosure, the means of drying is a water absorbent material.

In one embodiment of the disclosure, the means of drying is a water adsorbent material. In one embodiment of the disclosure, the means of drying is selected from the group consisting of molecular sieves, silica gel, alumina, bentonite clay, calcium oxide, an alkali metal carbonate, hydrogen carbonate, or an alkali earth metal carbonate.

In one embodiment, water is removed from the reaction mixture, such as to achieve a water content of about less than 2 wt % (relative to the weight of the reaction mixture), such as less than 2 wt %. In one embodiment, water is removed from the reaction mixture, such as to achieve a water content of about less than 1 wt % (relative to the weight of the reaction mixture), such as less than 1 wt %.

In additional or alternative embodiments the method described herein comprises a step of removing water from the reaction medium before, during or after the oxidation of the fatty alcohol. Said step can comprise adding a water absorbing or adsorbing material to the reaction medium absorbing or adsorbing water. Such water absorbing or adsorbing material includes but is not limited to molecular sieves, silica gels, aluminas, bentonite clays, calcium oxides, alkali metal carbonates, hydrogen carbonates, or alkali earth metal carbonates or a combination thereof. In some embodiments, the water absorbing or adsorbing material is selected from molecular sieves, silica gels, aluminas, bentonite clays, calcium oxides, alkali metal carbonates, hydrogen carbonates, or alkali earth metal carbonates or a combination thereof. The water absorbing or adsorbing material is suitably added to the reaction medium in amounts, so that the water content in the reaction medium after the oxidation process is 2% by weight or less, and optionally the molar conversion of fatty alcohol to fatty aldehydes is more than 93%. In some embodiments, optionally where the water absorbing or adsorbing material is a molecular sieve, the amount of water absorbing or adsorbing material added is at least 10 g per mmol of fatty alcohol present in the reaction medium prior to oxidation, such as at least 15 g per mmol of fatty alcohol, such as at least 19 g per mmol of fatty alcohol.

### Provision of Fatty Alcohol Compositions

The method disclosed herein may comprise an initial step of first producing the fatty alcohol composition as disclosed herein or producing the fatty alcohol as disclosed herein.

One embodiment of the present disclosure provides for a method as disclosed herein, wherein said method further comprises an initial step of producing the fatty alcohol, said initial step comprising the steps of:
i. providing a yeast cell capable of producing the fatty alcohol, and
ii. incubating said yeast cell in a medium,
   thereby producing the fatty alcohol.

One embodiment of the present disclosure provides for a method as disclosed herein, wherein said method further comprises an initial step of producing the fatty alcohol composition, said initial step comprising the steps of:
i. providing a yeast cell capable of producing the fatty alcohol composition, and
ii. incubating said yeast cell in a medium,
   thereby producing the fatty alcohol composition.

One embodiment of the present disclosure provides for a method as disclosed herein, wherein said method further comprises an initial step of producing the fatty alcohol, said initial step comprising the steps of:
i. providing a yeast cell capable of synthesising alkanoyl-CoA, said yeast cell further capable of expressing:
   a desaturase, and
   an alcohol-forming fatty acyl-CoA reductase,
ii. expressing said desaturase and said alcohol-forming fatty acyl-CoA reductase from said yeast cell, and
iii. incubating said yeast cell in a medium
   whereby the desaturase is capable of converting at least part of said alkanoyl-CoA to alkenoyl-CoA, and whereby said alcohol-forming fatty acyl-CoA reductase is capable of converting at least part of said alkenoyl-CoA to fatty alcohol, thereby producing said fatty alcohol. Details of carrying out the above production of fatty alcohol is disclosed in EP3313997 B1. In a further embodiment of the present disclosure, the fatty alcohol is (Z)-11-hexadecen-1-ol, wherein the alkanoyl-CoA is hexadecanoyl-CoA, wherein the desaturase is A11-desaturase, wherein the alkenoyl-CoA is (Z)-11-hexadecenoyl-CoA.

One embodiment of the present disclosure provides for a method as disclosed herein, wherein said method further comprises an initial step of producing the fatty alcohol composition, said initial step comprising the steps of:
i. providing a yeast cell capable of synthesising alkanoyl-CoA, said yeast cell further capable of expressing:
   a desaturase, and
   an alcohol-forming fatty acyl-CoA reductase,
ii. expressing said desaturase and said alcohol-forming fatty acyl-CoA reductase from said yeast cell, and
iii. incubating said yeast cell in a medium
   whereby the desaturase is capable of converting at least part of said alkanoyl-CoA to alkenoyl-CoA, and whereby said alcohol-forming fatty acyl-CoA reductase is capable of converting at least part of said alkenoyl-CoA to fatty alcohol, thereby producing said fatty alcohol composition. Details of carrying out the above production of fatty alcohol composition is disclosed in EP3313997 B1. In a further embodiment of the present disclosure, the fatty alcohol is (Z)-11-hexadecen-1-ol, wherein the alkanoyl-CoA is hexadecanoyl-CoA, wherein the desaturase is A11-desaturase, wherein the alkenoyl-CoA is (Z)-11-hexadecenoyl-CoA. Details on how to carry out the above steps of producing the fatty alcohol are disclosed in WO 2016/207339.

One embodiment of the present disclosure provides for a method as disclosed herein, wherein said method further comprises an initial step of producing the fatty alcohol, said initial step comprising the steps of:
i. providing an oleaginous yeast cell capable of producing a desaturated fatty alcohol, said yeast cell further:
   capable of expressing at least one heterologous fatty acyl-CoA desaturase capable of introducing at least one double bond in a fatty acyl-CoA, thus forming a desaturated fatty acyl-CoA,
   capable of expressing at least one heterologous fatty acyl-CoA reductase capable of converting at least part of said desaturated fatty acyl-CoA to a desaturated fatty alcohol,
   having a mutation resulting in reduced activity of a fatty alcohol oxidase and having a mutation resulting in reduced activity of at least one of: a fatty aldehyde dehydrogenase, a peroxisome biogenesis factor, and glycerol-3-phosphate acyltransferase, and
ii. incubating said yeast cell in a medium,
   thereby producing the fatty alcohol. Details on how to carry out the above steps of producing the fatty alcohol is disclosed in WO 2018/109163.

One embodiment of the present disclosure provides for a method as disclosed herein, wherein said method further comprises an initial step of producing the fatty alcohol composition, said initial step comprising the steps of:
i. providing an oleaginous yeast cell capable of producing a desaturated fatty alcohol, said yeast cell further:
   capable of expressing at least one heterologous fatty acyl-CoA desaturase capable of introducing at least one double bond in a fatty acyl-CoA, thus forming a desaturated fatty acyl-CoA,
   capable of expressing at least one heterologous fatty acyl-CoA reductase capable of converting at least part of said desaturated fatty acyl-CoA to a desaturated fatty alcohol,
   having a mutation resulting in reduced activity of a fatty alcohol oxidase and having a mutation resulting in reduced activity of at least one of: a fatty aldehyde dehydrogenase, a peroxisome biogenesis factor, and glycerol-3-phosphate acyltransferase, and
ii. incubating said yeast cell in a medium,
   thereby producing the fatty alcohol composition. Details on how to carry out the above steps of producing the fatty alcohol composition is disclosed in WO 2018/109163.

One embodiment of the present disclosure provides for a method as disclosed herein, wherein said method further comprises an initial step of producing the fatty alcohol, said initial step comprising the steps of:
i. providing a yeast cell capable of producing a desaturated fatty alcohol, said yeast cell expressing:
   at least one heterologous fatty acyl-CoA desaturase capable of introducing at least one double bond in a fatty acyl-CoA having a carbon chain length of 14, wherein said desaturase is selected from the group consisting of a A9 desaturase and a A11 desaturase, wherein the desaturase has a higher specificity towards tetradecanoyl-CoA then towards hexadecanoyl-CoA, and
   at least one heterologous fatty acyl-CoA reductase capable of converting at least part of said desaturated fatty acyl-CoA to a desaturated fatty alcohol, and
ii. incubating said yeast cell in a medium,
   thereby producing the fatty alcohol. Details on how to carry out the above steps of producing the fatty alcohol is disclosed in WO 2018/109167.

One embodiment of the present disclosure provides for a method as disclosed herein, wherein said method further comprises an initial step of producing the fatty alcohol composition, said initial step comprising the steps of:
i. providing a yeast cell capable of producing a desaturated fatty alcohol, said yeast cell expressing:
   at least one heterologous fatty acyl-CoA desaturase capable of introducing at least one double bond in a fatty acyl-CoA having a carbon chain length of 14, wherein said desaturase is selected from the group consisting of a 49 desaturase and a A11 desaturase, wherein the desaturase has a higher specificity towards tetradecanoyl-CoA then towards hexadecanoyl-CoA, and
   at least one heterologous fatty acyl-CoA reductase capable of converting at least part of said desaturated fatty acyl-CoA to a desaturated fatty alcohol, and
ii. incubating said yeast cell in a medium,
   thereby producing the fatty alcohol composition. Details on how to carry out the above steps of producing the fatty alcohol composition is disclosed in WO 2018/109167.

One embodiment of the present disclosure provides for a method as disclosed herein, wherein said method further comprises an initial step of producing the fatty alcohol composition, said initial step comprising the steps of:
i. providing a yeast cell capable of producing a desaturated fatty alcohol, said yeast cell expressing:
   a heterologous Δ12 fatty acyl-CoA desaturase, said desaturase being capable of introducing a double bond at position 12 in a saturated or desaturated fatty acyl-CoA, preferably a desaturated fatty acyl-CoA, having a carbon chain length of at least 13 and having n double bond(s),
   wherein n and n′ are integers,
   wherein 0 $ n $3 and wherein 1≤n′≤4, and
   at least one heterologous fatty acyl-CoA reductase capable of converting at least part of said desaturated fatty acyl-CoA to a desaturated fatty alcohol, and
ii. incubating said yeast cell in a medium,
   thereby producing the fatty alcohol composition. Details on how to carry out the above steps of producing the fatty alcohol composition are disclosed in application EP21183447.8 entitled “Methods and yeast cells for production of desaturated compounds” filed on 2 Jul. 2021 by same applicant.

One embodiment of the present disclosure provides for a method as disclosed herein, wherein said method further comprises an initial step of producing the fatty alcohol composition, said initial step comprising the steps of:
i. providing a yeast cell capable of producing a desaturated fatty alcohol, said yeast cell expressing:
   a heterologous Δ13 fatty acyl-CoA desaturase, said desaturase being capable of introducing a double bond at position 13 in a saturated or desaturated fatty acyl-CoA, preferably a desaturated fatty acyl-CoA, having a carbon chain length of at least 14 and having n double bond(s),
   wherein n and n′ are integers,
   wherein 0≤n≤3 and wherein 1≤n′≤4, and
   at least one heterologous fatty acyl-CoA reductase capable of converting at least part of said desaturated fatty acyl-CoA to a desaturated fatty alcohol, and
ii. incubating said yeast cell in a medium,
   thereby producing the fatty alcohol composition. Details on how to carry out the above steps of producing the fatty alcohol composition are disclosed in application EP21183459.3 entitled “Methods and yeast cells for production of desaturated compounds” filed on 2 Jul. 2021 by same applicant.

One embodiment of the present disclosure provides for a method as disclosed herein, wherein said method further comprises an initial step of producing the fatty alcohol composition, said initial step comprising the steps of:
i. providing a yeast cell capable of producing E8,E10-dodecadien-1-ol, said yeast cell expressing:
   at least one heterologous desaturase capable of introducing one or more double bonds in a fatty acyl-CoA having a carbon chain length of 12, thereby converting said fatty acyl-CoA to a desaturated fatty acyl-CoA, wherein at least part of said desaturated fatty acyl-CoA is E8,E10-dodecadienyl coenzyme A (E8,E10-C12:CoA), wherein:
   a) the at least one desaturase is Cpo CPRQ (accession number AHW98354), or a functional variant thereof having at least 80% identity thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity to Cpo_CPRQ; or
   b) the at least one desaturase is at least two desaturases, wherein at least one of said two desaturases is Cpo_CPRQ (accession number AHW98354), or a functional variant thereof having at least 80% identity thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity to Cpo_CPRQ, and the other desaturase is a desaturase capable of introducing at least one double bond in a fatty acyl-CoA having a carbon chain length of 12, such as a Z9-12 desaturase; and
   at least one heterologous fatty acyl-CoA reductase capable of converting at least part of said E8,E10-dodecadienyl coenzyme A to E8,E10-dodecadien-1-ol, and
ii. incubating said yeast cell in a medium,
   thereby producing the fatty alcohol composition. Details on how to carry out the above steps of producing the fatty alcohol composition are disclosed in application WO 2021/123128.

One embodiment of the present disclosure provides for method as disclosed herein, wherein the method further comprises an initial step of producing the fatty alcohol, said initial step comprising providing a yeast cell capable of producing the fatty alcohol and culturing said yeast cell in a culture medium under conditions allowing production of said fatty alcohol, wherein the culturing medium comprises an extractant in an amount equal to or greater than its cloud concentration measured in an aqueous solution such as the culture medium at the cultivation temperature, wherein the extractant is a non-ionic ethoxylated surfactant, thereby producing the fatty alcohol.

One embodiment of the present disclosure provides for method as disclosed herein, wherein the method further comprises an initial step of producing the fatty alcohol composition, said initial step comprising providing a yeast cell capable of producing the fatty alcohol composition and culturing said yeast cell in a culture medium under conditions allowing production of said fatty alcohol composition, wherein the culturing medium comprises an extractant in an amount equal to or greater than its cloud concentration measured in an aqueous solution such as the culture medium at the cultivation temperature, wherein the extractant is a non-ionic ethoxylated surfactant, thereby producing the fatty alcohol composition.

One embodiment of the present disclosure provides for a method as disclosed herein, wherein the method further comprises an initial step of producing the fatty alcohol, said initial step comprising
i. providing a yeast cell capable of producing a fatty alcohol ester, and culturing said yeast cell in a culture medium under conditions allowing production of said fatty alcohol ester, wherein the culturing medium comprises an extractant in an amount equal to or greater than its cloud concentration measured in an aqueous solution such as the culture medium at the cultivation temperature, wherein the extractant is a non-ionic ethoxylated surfactant, thereby producing the fatty alcohol ester, and
ii. converting said fatty alcohol ester to the fatty alcohol,
   thereby producing the fatty alcohol.

One embodiment of the present disclosure provides for a method as disclosed herein, wherein the method further comprises an initial step of producing the fatty alcohol composition, said initial step comprising
i. providing a yeast cell capable of producing a fatty alcohol ester, and culturing said yeast cell in a culture medium under conditions allowing production of said fatty alcohol ester, wherein the culturing medium comprises an extractant in an amount equal to or greater than its cloud concentration measured in an aqueous solution such as the culture medium at the cultivation temperature, wherein the extractant is a non-ionic ethoxylated surfactant, thereby producing the fatty alcohol ester, and
ii. converting said fatty alcohol ester to the fatty alcohol,
   thereby producing the fatty alcohol composition.

Details on how to perform cultivation of a yeast cell in a culture medium comprising an extractant in an amount equal to or greater than its cloud concentration are described in detail in application WO 2021/078452.

Additionally or alternatively, a composition is disclosed herein comprising more than 93% by weight fatty aldehyde, less than 7% by weight fatty alcohol and less than 2% by weight water. In some embodiments the amount of aldehyde can be 94% by weight or higher, such as 95% by weight or higher, such as 96% by weight or higher, such as 97% by weight or higher, such as 98% by weight or higher, such as at least 99% by weight, while the amount of non-converted fatty alcohol is less than 6% by weight, such as less than 5% by weight, such as less than 4% by weight, such as less than 3% by weight, such as less than 2% by weight, such as 1% by weight or less, while the amount of water is less than 2% by weight, such as less than 1.5% by weight, such as 1% by weight or less.

### Purification

The present disclosure provides a method of purifying fatty aldehydes, such as the fatty aldehydes disclosed herein, and fatty aldehyde compositions, such as the fatty aldehyde compositions disclosed herein.

One embodiment of the present disclosure provides for a fatty aldehyde purification method comprising the steps of:
a. providing a crude reaction product comprising:
   i. a fatty aldehyde,
   ii. copper ions, and
   iii. a polar solvent;
b. mixing said crude reaction product with an apolar, aprotic solvent and an acid to create an apolar phase and a polar phase; and
c. separating the apolar phase from the polar phase.

One embodiment of the present disclosure provides for a fatty aldehyde purification method as disclosed herein, wherein the crude reaction product comprises:
iv. 5 to 80% of the fatty aldehyde,
v. 0.05 to 5.0% copper ions,
vi. 20 to 95% of the polar solvent.

In some embodiments, the present disclosure provides for a method further comprising steps for purifying a fatty aldehyde comprising:
a) providing a purification mixture comprising:
   i. a fatty aldehyde,
   ii. copper ions, and
   iii. a polar solvent;
b) mixing said purification mixture with an apolar, aprotic solvent and an acid to create an extraction mixture comprising an apolar phase and a polar phase allowing the fatty aldehyde to be extracted from the polar phase to the apolar phase and
c) separating the apolar phase comprising purified aldehyde from the polar phase.

In some embodiments, the purification mixture comprises 0.05 to 5.0 wt % copper ions, such as 0.05 to 2.0 wt % copper ions, such as 0.05 to 1.0 wt % copper ions.

A representative embodiment of the crude reaction product as disclosed herein may comprise about 30% fatty aldehyde, about 1.0% ligand, about 0.4% copper, about 0.6% aminoxyl radical, about 0.5% base, and about 62% polar solvent. Another representative embodiment of the crude reaction product as disclosed herein may comprise about 30% fatty aldehyde, about 1.0% bipyridine, about 0.4% copper, about 0.6% 4-OH-TEMPO, about 0.5% 1-methylimidazole, and about 62% acetonitrile.

One embodiment provides for a fatty aldehyde purification method as disclosed herein wherein the crude reaction product comprises 0.05 to 5.0% copper ions, such as 0.05 to 2.0% copper ions, such as 0.05 to 1.0% copper ions. By a reference to a weight or weight percent of copper, copper ions, copper salt, and the like, as disclosed herein, it is meant that it is the weight content of the copper in isolation, i.e. without counter ion.

One embodiment provides for a fatty aldehyde purification method as disclosed herein wherein the crude reaction product further comprises a ligand, such as 0.1 to 10% of a ligand, such as 0.1 to 5% of a ligand, such as 0.1 to 2% of a ligand, such as about 1% of a ligand. In one embodiment of the present disclosure, the ligand is a bidentate nitrogen ligand, such as a bidentate nitrogen ligand selected from the group consisting of 2,2′-bipyridine, 4,4′-dimethyl-2,2′-bipyridine, 5,5′-dimethyl-2,2′-bipyridine 2,2′-bipyrimidine, 2,2′-bipyridine-4,4′-dicarboxylic acid or an ester thereof, 2,2′-bipyridine-5,5′-dicarboxylic acid or an ester thereof.

One embodiment provides for a fatty aldehyde purification method as disclosed herein wherein the crude reaction product further comprises an aminoxyl radical compound, such as 0.01 to 10% of an aminoxyl radical compound, such as 0.01 to 5% of an aminoxyl radical compound, such as about 0.01 to 2% of an aminoxyl radical compound, such as about 0.5% of an aminoxyl radical compound. In one embodiment of the present disclosure, the aminoxyl radical compound is selected from the group consisting of TEMPO, (4-hydroxy-2,2,6,6-tetramethylpiperidin-1-yl)oxyl (4-OH-TEMPO), 4-acetamido-TEMPO, 4-hydroxy-TEMPO benzoate, 4-amino-TEMPO, 2-azaadamantane-N-oxyl, 9-2-azaadamantane-N-oxyl, 4-oxo-TEMPO, and a polymer functionalised with any of said aminoxyl radical compounds.

One embodiment provides for a fatty aldehyde purification method as disclosed herein wherein the crude reaction product further comprises a base, such as 0.1 to 10% of a base, such as 0.1 to 5% of a base, such as 0.1 to 2% of a base, such as about 0.5% of a base. In one embodiment of the disclosure, the base is selected from the group consisting of 1-methylimidazole, 1,8-diazabicyclo[5.4.0]undec-7-ene, 1,5-diazabicyclo[4.3.0]non-5-ene, 1,5,7-triazabicyclo[4.4.0]dec-5-ene, 1,1,3,3-tetramethylguanidine, 7-methyl-1,5,7-triazabicyclo[4.4.0]dec-5-ene, and potassium t-butoxide.

In one embodiment of the present disclosure, the fatty aldehyde is a saturated fatty aldehyde as disclosed herein. In one embodiment of the present disclosure, the fatty aldehyde is an unsaturated fatty aldehyde as disclosed herein. In one embodiment of the present disclosure, the fatty aldehyde is as disclosed in the section “fatty aldehydes”. In one embodiment, the fatty aldehyde is selected from the group consisting of: (E)7,(Z)9 desaturated fatty aldehyde having a carbon chain length of 14, (E)3,(Z)8,(Z)11 desaturated fatty aldehyde having a carbon chain length of 14, (Z)9,(E)11,(E)13 desaturated fatty aldehyde having a carbon chain length of 14, (E)7,(Z)9 desaturated fatty aldehyde having a carbon chain length of 12, (E)3,(Z)8,(Z)11 desaturated fatty aldehyde having a carbon chain length of 12, (Z)9,(E)11,(E)13 desaturated fatty aldehyde having a carbon chain length of 12, and (E)8,(E)10 desaturated fatty aldehyde having a carbon chain length of 12. In one embodiment, the fatty aldehyde is selected from the group consisting of tetradecan-1-al, pentadecan-1-al, hexadecan-1-al, pentadecen-1-al, (Z)-9-hexadecen-1-al, (Z)-11-hexadecen-1-al, and (7E,9E)-undeca-7,9-dien-1-al.

In one embodiment, the copper ions are copper (I) and/or copper (II) ions. In one embodiment, the copper ions are copper (II) ions.

In one embodiment, the polar solvent is selected from the group consisting of acetonitrile, dimethylformamide, acetonitrile, propionitrile, butyronitrile, dimethyl sulfoxide, dimethyl acetamide, and propylene carbonate. In one embodiment, the polar solvent is acetonitrile.

In one embodiment, the apolar, aprotic solvent is selected from the group consisting of linear alkanes, branched alkanes, and cycloalkanes. In one embodiment, the apolar, aprotic solvent is selected from the group consisting of pentanes, hexanes, heptanes, and octanes. In one embodiment, the apolar, aprotic solvent is selected from the group consisting of heptane, pentane, hexane, cyclohexane, and octane.

In one embodiment, the acid has a pKa value between 3 and 6. In one embodiment, the acid is a carboxylic acid. In one embodiment, the carboxylic acid is a C₂-C₈ carboxylic acid. In one embodiment, the carboxylic acid is selected from the group consisting of C₂-C₈ monocarboxylic acids, C₂-C₈ dicarboxylic acids, and C₆-C₈ tricarboxylic acids. In one embodiment, the carboxylic acid is selected from the group consisting of acetic acid, citric acid, propanoic acid, lactic acid, glycolic acid, poly acrylic acid. In one embodiment, at least 1.0 molar equivalents of carboxylic acid relative to copper is used. In one embodiment, at least 2.0 molar equivalent of carboxylic acid relative to the copper is used, such as at least 2.4 equivalents. In one embodiment, 2.0 to 2.4, 2.4 to 2.8, 2.8 to 3.2, 3.2 to 3.6, 3.6 to 4.0, 4.0 to 5.0, 5.0 to 6.0, 6.0 to 7.0, 7.0 to 8.0, 8.0 to 9.0, 9.0 to 10.0, or more than 10.0 equivalents of carboxylic acid relative to the copper is used. By “equivalents” is meant molar equivalents.

In one embodiment of the present disclosure, the crude reaction product further comprises an oxidising agent and/or a spent oxidising agent. In one embodiment, the oxidising agent or the spent oxidising agent is selected from the group consisting of TEMPO, (4-hydroxy-2,2,6,6-tetramethylpiperidin-1-yl)oxyl (4-OH-TEMPO), 4-acetamido-TEMPO, 4-hydroxy-TEMPO benzoate, 4-amino-TEMPO, 2-azaadamantane-N-oxyl, 9-azabicyclo[3.3.1]nonane N-oxyl, 4-carboxy-TEMPO, 4-maleimido-TEMPO, 4-methoxy-TEMPO, 1-methyl-2-azaadamantane-N-oxyl, 4-oxo-TEMPO, and a polymer functionalised with any of said aminoxyl radical compounds; or spent agents thereof, or water.

In one embodiment, the fatty aldehyde purification methods as disclosed herein further comprising a step of evaporating the apolar, aprotic solvent. In one embodiment, the evaporation of the apolar, aprotic solvent is performed at reduced pressure, such as below 100 mbar, such as below 50 mbar, such as below 40 mbar, such as below 30 mbar.

One embodiment of the present disclosure provides for a method of converting a composition comprising a fatty alcohol to a composition enriched in fatty aldehyde, said method comprising:
a. converting the composition comprising a fatty alcohol to a composition comprising a fatty aldehyde using the method of oxidising fatty alcohols disclosed herein, and
b. purifying said composition comprising a fatty aldehyde using a the fatty aldehyde purification method disclosed herein.
   In a further embodiment, the fatty alcohol and the fatty aldehyde are desaturated.

In one exemplary embodiment of the disclosure, the crude reaction product containing catalyst system (Cupper complex, TEMPO or derivative, N-methyl-imidazole or another base) the product(s) from the oxidation to fatty aldehyde, and any unreacted alcohol(s), is dissolved or suspended in acetonitrile or another highly polar solvent such as dimethyl formamide, dimethyl sulfoxide or similar. The reaction mixture is then extracted with an organic solvent that is immiscible with the reaction solvent, typically an alkane such as pentane, heptane or hexane. The extraction can be using a separating funnel, mixer settler, pulse column or any other method for liquid-liquid separation. The settling of the phases occurs rapidly without any formation of foams or suspensions. The heavy phase contains nearly all catalyst components, and the reaction products are almost exclusively in the light phase. Evaporation of the extraction solvent yields the product fatty aldehyde. Optionally an additive can be included to improve removal of a one or more components, such as copper ions. Such additive may be an organic acid, such as acetic acid or citric acid.

### Products

One embodiment of the present disclosure provides for a composition comprising a fatty aldehyde obtained from the method disclosed herein. One embodiment of the disclosure provides for a fatty aldehyde obtained from the method disclosed here. In a further embodiment, the fatty aldehyde is desaturated.

Copper ions in solution (aqueous or non-aqueous) often have a blue colour. In one embodiment of the present disclosure, the composition exhibits an absorption at 680 nm of at most 0.5 in a cuvette having a 5 mm path length. In one embodiment, the absorption at 680 nm is at most 0.4, such as at most 0.3, such as at most 0.2, such as at most 0.1, such as at most 0.08, such as at most 0.06, such as at most 0.05 in a cuvette having a 5 mm path length. In one embodiment, the fatty aldehyde composition comprises less than 0.4% copper, such as less than 0.3%, such as 0.2%, such as less than 0.1%, such as less than 0.08%, such as less than 0.06%, such as less than 0.05%, such as less than 0.04%.

The presently disclosed fatty aldehydes may be produced from renewable feedstocks. The fatty aldehydes, or any of the slow release compositions thereof, act as pheromone components. Thus, one embodiment of the present disclosure provides for a pheromone component produced from renewable feedstocks. One embodiment of the present disclosure provides for a pheromone component produced from renewable feedstocks, said pheromone component having at least than 80% of biobased carbon content. In one embodiment, by “biobased carbon” content is meant organic compounds wherein the carbon originates from biological sources or precursors. In one embodiment, the pheromone component comprises the fatty aldehyde composition and/or the fatty aldehyde as disclosed herein. In one embodiment, the pheromone component comprises the slow release composition as disclosed herein. In one embodiment, the pheromone component comprises the fatty acetal and/or the α-hydroxysulfonic acid as disclosed herein.

In some embodiments, a composition is provided comprising more than 93% by weight fatty aldehyde, less than 7% by weight fatty alcohol and less than 2% by weight water, optionally free/unbound water.

In some embodiments, the composition is provided wherein the light absorption at 680 nm is at most 0.4, such as at most 0.3, such as at most 0.2, such as at most 0.1, such as at most 0.08, such as at most 0.06, such as at most 0.05 in a cuvette having a 5 mm path length.

### Method of Producing Fatty Acetals and α-Hydroxysulfonic Acids.

Fatty aldehydes may feasibly be converted to other compounds which are capable of converting back to said fatty aldehydes. Such conversion back to fatty aldehyde may be via hydrolysis of bonds, cleavage of bonds, and/or conversion of functional groups. Such other compounds may serve to better store the fatty aldehydes, releasing the fatty aldehyde gradually as the compound converts back. For example, said compounds may be less volatile than the corresponding fatty aldehyde, whereby the more volatile fatty aldehydes are continuously released as the compounds converts to fatty aldehyde. Suitable compounds which can be produced from fatty aldehydes include acetals and α-hydroxysulfonic acids.

One embodiment of the present disclosure provides for a method of converting a fatty alcohol to a fatty acetal, said method comprising the steps of:
a. providing a reaction mixture comprising a fatty alcohol composition comprising the fatty alcohol as disclosed herein, a catalyst composition as disclosed herein, and a solvent as disclosed herein
b. exposing the reaction mixture to at least 0.25 ml oxygen per minute per gram of fatty alcohol by means of bubbling a gas mixture comprising oxygen through the reaction mixture, thereby obtaining a fatty aldehyde, and
c. converting the aldehyde functional groups of the fatty aldehyde to acetal functional groups,
   thereby obtaining the fatty acetal. In a further embodiment, the fatty alcohol is a desaturated fatty alcohol. In yet a further embodiment, the fatty aldehyde is a desaturated fatty aldehyde. In yet a further embodiment, the fatty acetal is a desaturated fatty acetal.

One embodiment of the present disclosure provides for a method of converting a fatty alcohol to a fatty acetal, said method comprising the steps of:
a. converting the fatty alcohol to a fatty aldehyde as disclosed herein, and
b. converting the aldehyde functional groups of the fatty aldehyde to acetal functional groups,
   thereby obtaining the fatty acetal. In a further embodiment, the fatty alcohol is a desaturated fatty alcohol. In yet a further embodiment, the fatty aldehyde is a desaturated fatty aldehyde. In yet a further embodiment, the fatty acetal is a desaturated fatty acetal.

One embodiment provides for a fatty acetal obtained from the method disclosed herein.

One embodiment of the present disclosure provides for a method of converting a fatty alcohol to a fatty α-hydroxysulfonic acid, said method comprising the steps of:
a. providing a reaction mixture comprising a fatty alcohol composition comprising the fatty alcohol as disclosed herein, a catalyst composition as disclosed herein, and a solvent as disclosed herein,
b. exposing the reaction mixture to at least 0.25 ml oxygen per minute per gram of fatty alcohol by means of bubbling a gas mixture comprising oxygen through the reaction mixture, thereby obtaining a fatty aldehyde, and
c. converting the aldehyde functional groups of the fatty aldehyde to α-hydroxysulfonic acid functional groups,
   thereby obtaining the fatty α-hydroxysulfonic acid. In a further embodiment, the fatty alcohol is a desaturated fatty alcohol. In yet a further embodiment, the fatty aldehyde is a desaturated fatty aldehyde. In a further embodiment, the fatty α-hydroxysulfonic acid is a desaturated fatty α-hydroxysulfonic acid.

One embodiment of the present disclosure provides for a method of converting a fatty alcohol to a fatty α-hydroxysulfonic acid, said method comprising the steps of:
a. converting the fatty alcohol to a fatty aldehyde as disclosed herein, and
b. converting the aldehyde functional groups of the fatty aldehyde to α-hydroxysulfonic acid functional groups,
   thereby obtaining the fatty α-hydroxysulfonic acid. In a further embodiment, the fatty alcohol is a desaturated fatty alcohol. In yet a further embodiment, the fatty aldehyde is a desaturated fatty aldehyde. In a further embodiment, the fatty α-hydroxysulfonic acid is a desaturated fatty α-hydroxysulfonic acid.

One embodiment of the disclosure provides for a fatty α-hydroxysulfonic acid obtained from the method disclosed herein.

### Pheromones and Controlled Release Thereof

The presently disclosed compounds may act as pheromones. In one embodiment of the present disclosure, the pheromone composition as disclosed herein may comprise one or more aldehydes as disclosed herein, one or more acetals as disclosed herein, and/or one or more α-hydroxysulfonic acids as disclosed herein. In a specific embodiment of the present disclosure, the pheromone composition as disclosed herein may comprise one or more fatty aldehydes as disclosed herein, one or more fatty acetals as disclosed herein, and/or one or more fatty α-hydroxysulfonic acids as disclosed herein.

It can be advantageous to control the release pheromones from a pheromone composition in order to control the concentration of pheromones in the air, for example the air over crops. Pheromone compositions can be formulated to provide slow release into the atmosphere, and/or to be protected from degradation following release. In one embodiment of the present disclosure, the pheromone compositions are included in carriers such as microcapsules, biodegradable flakes, or paraffin wax-based matrices. In one embodiment of the present disclosure the pheromone composition is formulated as a slow release sprayable.

In certain embodiments, the pheromone composition may include one or more polymeric agents known to one skilled in the art, to control the release of the composition to the environment. In some embodiments, the polymeric attractant-composition is impervious to environmental conditions. The polymeric agent may also be a sustained-release (or slow release or controlled release) agent that enables the pheromone composition to be continuously released to the environment. In one embodiment of the present disclosure, the polymeric agent is selected from the group consisting of cellulose, cellulose derivatives, proteins such as casein, fluorocarbon-based polymers, hydrogenated rosins, lignins, melamine, polyurethanes, vinyl polymers such as polyvinyl acetate (PVAC), polycarbonates, polyvinylidene dinitrile, polyamides, polyvinyl alcohol (PVA), polyamide-aldehyde, polyvinyl aldehyde, polyesters, polyvinyl chloride (PVC), polyethylenes, polystyrenes, polyvinylidene, silicones, and combinations thereof. In one embodiment of the disclosure, the cellulose derivative is selected from the group consisting of methylcellulose, ethyl cellulose, cellulose acetate, cellulose acetate-butyrate, cellulose acetate-propionate, cellulose propionate, and combinations thereof.

In one embodiment of the present disclosure, the sustained-release pheromone composition comprises one or more fatty acid esters or one or more fatty alcohol. In one embodiment of the present disclosure, the one or more fatty alcohols is selected from the group consisting of undecanol, dodecanol, tridecanol, tridecenol, tetradecanol, tetradecenol, tetradecadienol, pentadecanol, pentadecenol, hexadecanol, hexadecenol, hexadecadienol, octadecenol and octadecadienol). In one embodiment of the present disclosure the fatty acid ester is selected from the group consisting of an undecanyl ester, a dodecanyl ester, a tridecanyl ester, a tridecenyl ester, a tetradecanyl ester, a tetradecenyl ester, a tetradecadienyl ester, a pentadecanyl ester, a pentadecenyl ester, a hexadecanyl ester, a hexadecenyl ester, a hexadecadienyl ester, an octadecenyl ester, and an octadecadienyl ester. In one embodiment of the present disclosure, the fatty acid ester is selected from the group consisting of an alkyl undecanoate, an alkenyl undecanoate, an alkyl dodecanoate, an alkenyl dodecanoate, an alkyl tridecanoate, an alkenyl tridecanoate, an alkyl tridecenoate, an alkenyl tridecenoate, an alkyl tetradecanoate, an alkenyl tetradecanoate, an alkyl tetradecenoate, an alkenyl tetradecenoate, an alkyl tetradecadienoate, an alkenyl tetradecadienoate, an alkyl pentadecanoate, an alkenyl pentadecanoate, an alkyl pentadecenoate, an alkenyl pentadecenoate, an alkyl hexadecanoate, an alkenyl hexadecanoate, an alkyl hexadecenoate, an alkenyl hexadecenoate, an alkyl hexadecadienoate, an alkenyl hexadecadienoate, an alkyl octadecenoate, an alkenyl octadecenoate, an alkyl octadecadienoate, and an alkenyl octadecadienoate.

An alternative method for controlling the release of pheromones is to use a compound that degrades to the active pheromone when subjected to the elements. Aldehydes such as the fatty aldehydes disclosed herein readily undergoes reaction with alcohols to form dialkyl acetals. The alcohol can be another pheromone alcohol (e.g. Z11-hexanedecen-1-ol, Z9-hexanedecen-1-ol or similar unsaturated alcohol). Alternatively, the alcohol can be a short chain alcohol such as methanol, ethanol, propan-1-ol, propan-2-ol, butan-1-ol, and butan-2-ol. Moreover, a cyclic acetal might be formed when the alcohol is a diol. Examples of diols are ethylene glycol, 1,3-propylene glycol and 1,2-propylene glycol. Alternatively, a mixture of alcohols can be used. A fatty acetal may be produced using a method as disclosed herein. In one embodiment of the disclosure, the fatty acetal is produced from a fatty aldehyde as disclosed herein and two similar or different alcohols. In one embodiment, the acetal is produced from a fatty aldehyde as disclosed herein and two similar or different alcohols as disclosed herein. In one embodiment of the present disclosure, the fatty acetal is produced from a fatty aldehyde as disclosed herein and two similar or different fatty alcohols as disclosed herein. In one embodiment, the fatty acetal is produced from a fatty aldehyde as disclosed herein and two similar or different C1-C7 alcohols. In one embodiment of the present disclosure, the fatty acetal is produced from a fatty aldehyde as disclosed herein and two similar or different alcohols selected from the group consisting of methanol, ethanol, propan-1-ol, propan-2-ol, butan-1-ol, and butan-2-ol. In one embodiment of the disclosure, the acetal is produced from a fatty aldehyde as disclosed herein and a diol. In one embodiment, the acetal is produced from a fatty aldehyde as disclosed herein and a diol selected from the group consisting of ethylene glycol, 1,3-propylene glycol, and 1,2-propylene glycol.

The term “produced from” in relation to acetals is not intended to limit the acetal to the specific method of its production. The term is merely used to provide structural information about the acetal. For example, acetals can be produced from the reaction of a hemiacetal with one alcohol. By way of example, an acetal produced from 1-methoxyethan-1-ol (a hemiacetal) is equivalent to an acetal produced from ethanol and two molecules of methanol.

The fatty aldehydes of the present disclosure may also be present in the pheromone composition as oligomeric cyclic compounds. Accordingly, in one embodiment of the present disclosure, the fatty aldehyde is present in the pheromone composition as a trioxane and/or a tetraoxane. These oligomers acts as reservoirs of the aldehyde, allowing for the controlled release of the aldehyde. Aldehyde oligomers can be produced in the presence of an acid catalyst. Examples of acids which may be used in acetal formation or oxane formation include hydrochloric acid, sulphuric acid phosphoric acid or hydrogen sulfate salts. Hydrogen sulfate salts is particularly advantageous for formation of oxanes.

Another suitable slow release compound is α-hydroxysulfonic acids. These compounds are readily formed from an aqueous solution of a hydrogen sulfite salt, particularly sodium hydrogen sulfite. In one embodiment of the present disclosure, the sustained release pheromone composition comprises an α-hydroxysulfonic acid.

Above mentioned slow release pheromones of acetal type gradually revert to aldehydes in when exposed to mild acids and moisture. The rate of release will depend on the type nature of acetals allowing custom compositions adapted for certain environments. The α-hydroxysulfonic acids slow release pheromone both acidic and alkaline condition.

One embodiment of the present disclosure provides for a fatty aldehyde slow-release composition comprising the fatty acetal disclosed herein. One embodiment of the disclosure provides for a method of producing the fatty aldehyde slow-release composition disclosed herein, said method comprising carrying out the method disclosed herein to provide a fatty acetal and formulating said fatty acetal in a slow-release composition. In a further embodiment, said fatty aldehyde is a desaturated fatty aldehyde. In yet a further embodiment, said fatty acetal is a desaturated fatty acetal.

One embodiment of the present disclosure provides for a fatty aldehyde slow-release composition comprising the fatty α-hydroxysulfonic acid disclosed herein. One embodiment of the disclosure provides for a method of producing the fatty aldehyde slow-release composition disclosed herein, said method comprising carrying out the method disclosed herein to provide a fatty α-hydroxysulfonic acid and formulating said fatty α-hydroxysulfonic acid in a slow-release composition. In a further embodiment, said fatty aldehyde is a desaturated fatty aldehyde. In yet a further embodiment, said fatty α-hydroxysulfonic acid is a desaturated fatty acetal.

The slow release pheromones can optionally be formulated with agents to further modify the release of compounds. These compositions optionally include agents to regulate moisture and pH. The slow release composition can be a mixture of above-mentioned acetals, aldehydes, alcohols, and α-hydroxysulfonic acids.

It is advantageous to minimise the number of steps in a chemical synthesis to for example reduce cost and minimize waste. It is advantageous that the acetals and α-hydroxysulfonic acids disclosed herein are produced in as few steps as possible. This may be achieved by producing the acetals and α-hydroxysulfonic acids directly from the reaction mixture used for production of fatty aldehydes.

### Biological Production of Fatty Alcohols

Methods of producing desaturated fatty alcohols, desaturated fatty alcohol acetates and desaturated fatty aldehydes in microbial cell factories, in particular in yeast, are available in the art.

In particular, the desaturated compounds may be obtained as described in WO 2016/207339, WO 2018/109163, WO 2018/109167, WO 2021/078452, WO 2020/169389, WO 2021/123128 and in applications EP21183447.8 entitled “Methods and yeast cells for production of desaturated compounds” filed on 2 Jul. 2021 by same applicant, and EP21183459.3 entitled “Methods and yeast cells for production of desaturated compounds” filed on 2 Jul. 2021 by same applicant.

In short, desaturated fatty alcohols can be produced in a yeast cell, in particular in a Saccharomyces or Yarrowia cell, such as a Saccharomyces cerevisiae or a Yarrowia lipolytica cell, by introducing one or more suitable heterologous fatty acyl-CoA desaturases, which introduce at least one double bond in a fatty acyl-CoA, and one or more suitable heterologous fatty acyl reductases (FAR). These desaturated fatty alcohols can then be converted to desaturated fatty aldehydes using the methods disclosed herein.

### EXAMPLES

### Example 1: Oxidation of a Mixture of Fatty Alcohols with Low Oxygen Transfer Rate

To 800 g of fatty alcohol mixture (Table 1) 1755 g of acetonitrile was added. To the reaction mixture a catalyst comprising of 26.2 g 2.2′-bipyridine, 62.7 g of tetrakisacetonitrile copper (I) triflate, 17.5 g of 4-hydroxy-TEMPO and 13.8 g 1-methylimidazol. The reaction was started with airflow of 1 dm3/min which was reduced to 0.2 dm3/min after 160 minutes. The conversion of alcohol to aldehyde levelled out at 60%.

### Reaction Sampling

1 ml reaction mixture was removed from the reactor and quenched with 1 ml saturated NaHCO3, 1 ml Ethyl acetate was added, and the sample shaken vigorously. 1 μl of the organic phase was removed and diluted in 1 ml ethyl acetate in a GC vial. The sample was analysed by GC-FID. For reaction monitoring the relative peak area % was used to ascertain the progress of the reaction. Reaction sampling was carried out similarly in the following examples.

TABLE 1
Composition of feed for Example 1.

| Compound | Avg % (w/w) |
|---|---|
| Tetradecan-1-ol | 0.9 |
| (Z)-11-Hexadecenal | 0.4 |
| (Z)-9-Hexadecen-1-ol | 3.9 |
| (Z)-11-Hexadecen-1-ol | 73.8 |
| Hexadecan-1-ol | 6.2 |
| Total Quantified by GC-FID | 85.2 |

### Example 2: Oxidation of a Mixture of Fatty Alcohols with Intermediate Oxygen Transfer Rate

To 800 g of fatty alcohol mixture 1755 g of acetonitrile was added. To the reaction mixture a catalyst comprising of 26.2 g 2.2′-bipyridine, 62.7 g of tetrakisacetonitrile copper (I) triflate, 17.5 g of 4-hydroxy-TEMPO and 13.8 g N-methylimidazol. The reaction was started with airflow of 1 dm3/min maintained at 1 dm3/min. The conversion of alcohol to aldehyde levelled out at 83%.

### Example 3: Oxidation of a Mixture of Fatty Alcohols with High Oxygen Transfer Rate

To 100 g of fatty alcohol mixture 218 g of acetonitrile was added. To the reaction mixture a catalyst comprising of 1.56 g 2.2′-bipyridine, 3.77 g of tetrakisacetonitrile copper (I) triflate, 1.03 g of 4-hydroxy-TEMPO and 0.82 g N-methylimidazol. The reaction was started with airflow of 1 dm3/min maintained at 1 dm3/min. The conversion of alcohol to aldehyde levelled out at 93%.

### Example 4: Oxidation of a Mixture of Fatty Alcohols

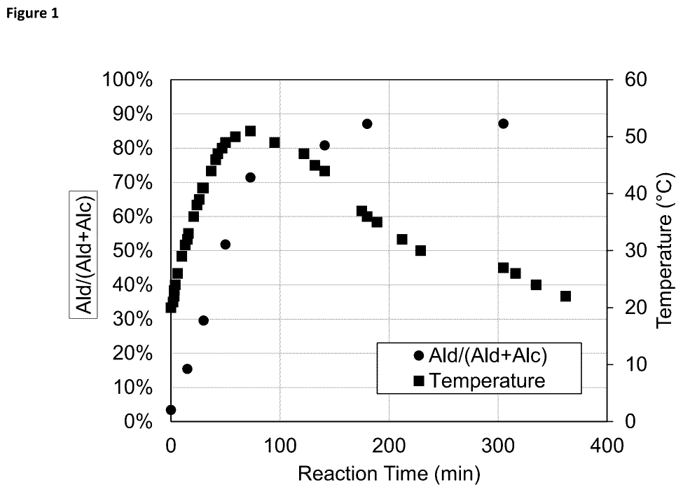

A mixture of fatty alcohols 800 g comprising of fatty alcohols was oxidised using air bubbled through solution of above-mentioned fatty alcohol mix and acetonitrile 1600 ml at a rate of 2.2 dm3/min. To the reaction mixture, a catalyst comprising of 62 g tetrakisacetonitrilecopper (I) trifluoromethane sulfonate, 26 g 2.2′-bipyridine, 10 g 4-hydroxy TEMPO, and 13.6 g 1-methyl-imidazole was added. The reaction was left for 2 h during which the temperature increased from 22° C. to 52° C. after 1 h followed by a drop in temperature to 42° C. after 2 h. The reaction yield increased steadily to over 70% after 73 min, and increased further to 87% at 150 min. FIG. 1 shows the reaction yield as a function of time.

### Example 5: Oxidation of a Mixture of Fatty Aldehydes Using an Adsorbent to Adsorb Reaction Water

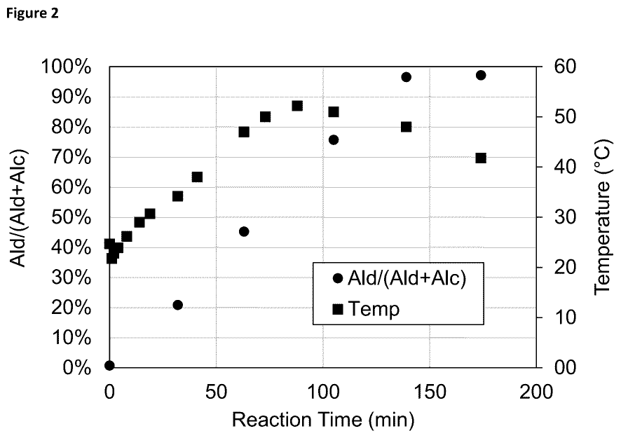

A mixture of fatty alcohols 252 g comprising of fatty alcohols in Table 2 was oxidised using air bubbled through solution of above mentioned fatty alcohol mix and acetonitrile 625 g at a rate of 2 dm³/min. To the reaction mixture a catalyst comprising of 18 g tetrakisacetonitrilecopper (I) trifluoromethane sulfonate, 8.2 g 2.2′-bipyridine, 5.5 g 4-hydroxy TEMPO, 8.5 g 1-Methyl-imidazole and 20 g 4 Å molecular sieves were added. The reaction was left for 2 h during which the temperature increased from 22° C. to 52° C. after 1 h followed by a drop in temperature to 42° C. after 2 h. The conversion increased steadily to over 95% at 139 min. FIG. 2 shows the reaction yield as a function of time.

TABLE 2
Composition of alcohol mixture used in example.

| Compound | Avg, % (w/w) |
|---|---|
| Monounsaturated tetradecen-1-ol | <LOQ |
| Tetradecan-1-ol | 1.4 |
| Monounsaturated pentadecen-1-ol | 8.1 |
| Pentadecan-1-ol | 1.6 |
| (Z)-9-Hexadecen-1-ol | 3.5 |
| (Z)-11-Hexadecen-1-ol | 66.6 |
| Hexadecan-1-ol | 7.0 |
| Total Quantified by GC-FID | 88.1 |

### Example 6: Oxidation of a Mixture of Fatty Alcohols Using Water Adsorbent to Adsorb Water from Solvent and Reaction Water

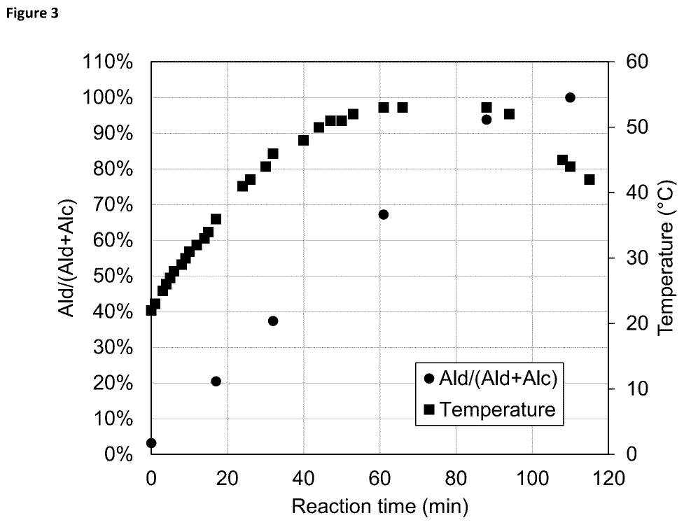

A mixture of fatty alcohols 800 g comprising of fatty alcohols in table 3 was oxidised using air bubbled through solution of above mentioned fatty alcohol mix and acetonitrile 1766 g at a rate of 6 dm3/min. To the reaction g mixture a catalyst comprising of 62 tetrakisacetonitrilecopper (I) trifluoromethane sulfonate, 26 g 2.2′-bipyridine, 10.6 g 4-hydroxy TEMPO, 16.6 g N-imidazole and 65 g 4 Å molecular sieves were added. The reaction was left for 2 h during which the temperature increased from 23° C. to 51° C. after 1 and 13 min h followed by a drop in temperature to 22° C. after 6 h. The conversion increased steadily to over 99% at 110 min. FIG. 3 shows the reaction yield as a function of time.

TABLE 3
Composition of alcohol mixture used in Example 6.

| Compound | Avg, % (w/w) |
|---|---|
| Monounsaturated tetradecen-1-ol | <LOQ |
| Tetradecan-1-ol | <LOQ |
| Monounsaturated pentadecen-1-ol | 5.9 |
| Pentadecan-1-ol | 0.8 |
| (Z)-11-Hexadecenal | <LOQ |
| (Z)-9-Hexadecen-1-ol | 3.2 |
| (Z)-11-Hexadecen-1-ol | 78.2 |
| Hexadecan-1-ol | 7.6 |
| Other Fatty alcohols | <6% |
| Total Quantified by GC-FID | >99% |

### Example 7: Comparison of Low, Intermediate, and High Oxygen Transfer Rate

Fatty alcohol composition, catalyst composition, and solvent were mixed as outlined in the preceding examples. Table 4 outlines the conversion of fatty alcohol to fatty aldehyde given as ald/(alc+ald), provided the indicated sparging rates and sparging time. A 20% oxygen gas mixture was used.

TABLE 4
conversion of fatty alcohol to fatty aldehyde

| | Final | | | | Sparging per |
|---|---|---|---|---|---|
| | Ald/ | | Sparging | Starting | mass alcohol |
| | (Alc + | Time | dm³ × | alcohol | dm³ × kg⁻¹ × |
| Example | Ald) | (min) | min⁻¹ | kg | min⁻¹ |
| 1 | 60% | 1427 | 0.2 | 0.80 | 0.25 |
| 2 | 83% | 1365 | 1 | 0.80 | 1.25 |
| 3 | 93% | 320 | 2.2 | 0.1 | 22 |
| 4 | 87% | 305 | 2.2 | 0.8 | 2.75 |

Low oxygen transfer rate (sparging 0.2 L/h) provided only moderate conversion of fatty alcohol to fatty aldehyde at long reaction time (1427 min). Intermediate oxygen transfer rate (1 L/h) provided better conversion but also required a long reaction time. High oxygen transfer rate (2.2 L/h) provided for excellent conversion of fatty alcohol. There was a tendency that long reaction time (305-320 min) lead to somewhat lower yields of 87-93%, whereas shorter reaction time (110-174 min) lead to excellent conversion of 97-99%.

Furthermore, it was observed that, removal of water formed during the reaction further improved the yield (Table 5). It is known that water is involved in the formation of carboxylic acids and thus removal of water improves yields further. It should be noted that not all water needs to be adsorbed, it is sufficient to add enough molecular sieves to reduce the final water content by ca 16 mol %.

TABLE 5
Improved reaction yields by partial water removal during oxidation.

| | | | | | Sparging | |
|---|---|---|---|---|---|---|
| | | | | | per mass | |
| | | | | | alcohol | Molecular |
| | Final Ald/ | Time | Sparging | Starting | dm³ × kg⁻¹ × | sieves |
| Example | (Alc + Ald) | (min) | dm³ × min⁻¹ | alcohol kg | min⁻¹ | (g) |
| 4 | 87% | 305 | 2.2 | 0.8 | 2.75 | 0 |
| 5 | 97% | 174 | 2 | 0.25 | 8 | 20 |
| 6 | 99% | 110 | 6 | 0.8 | 7.510 | 64 |

### Example 8: Purification of Reaction Mixture

### Methods

Calibration standards containing dodecan-1-ol, (Z)-11-hexadecenal, (Z)-9-hexadecenal, hexadecanal, tetradecanal, tetradecan-1-ol, pentadecanal, pentadecan-1-ol, hexadecan-1-ol, (Z)-9-hexadecan-1-ol and (Z)-11-hexadecan-1-ol with a concentration range of 0.01 mg/ml to 1 mg/ml were prepared. 10 μl of 10 mg/ml methyl-nonadecanoate was added to 1 mL standard and a calibration curve was obtained. Monounsaturated pentadecenal was quantified with pentadecanal.

UV-vis spectrometer: Thermo Genesys 5S was used for UV-vis spectrometry. Sample was put in concentrated form in 5 mm quartz cuvettes and a spectrum is measured from 350 nm to 1100 nm. λmax for Cu adsorption is measured at 680 nm.

### Sample Preparation for GC-FID:

Three aliquots of the sample of ca. 50 mg each were individually transferred to 50-mL volumetric flasks, weighed, and ethyl acetate was added until the volume mark. 1000 μL of each diluted aliquot was transferred into a GC vial, and 10 μl of internal standard solution was added. Internal standard solution was 10 mg/ml methyl-nonadecanoate in ethyl acetate.

### Analytical Conditions:

Qualitative analysis was carried out on an Agilent GC 7820A coupled to MS 5977B, split/spitless injector and a DB-Fatwax Ul column (30 m, 0.25 mm i.d. and 0.25 μm film). The operation parameters were: 1 μL injection, split ratio 20:1, injector temperature 220° C., constant flow 1 mL/min Helium, oven ramp 80° C. for 1 min, 15° C./min to 150° C. for 7 min, 10° C./min to 210° C. for 7 min and 20° C./min to 230° C. for 5 min.

Quantitative analysis was carried out on an Agilent GC 7890B coupled to FID, split/spitless injector and an HP-5 column (30 m, 0.32 mm i.d. and 0.25 μm film). The operation parameters were: 1 μL injection, split ratio 1:40, injector temperature 220° C., constant flow 2 mL/min hydrogen, oven ramp 80° C. for 1 min, 15° C./min to 150° C. for 7 min, 10° C./min to 210° C. and 20° C./min to 300° C.

### Analytical Standards:

(Z)-11-hexadecenal from Pherobank had a purity of 99.1%. Pentadecan-1-ol from Alfa Aesar was 99% pure. Hexadecan-1-ol was purchased from Merck with a purity of 99%. (Z)-9-hexadecen-1-ol and (Z)-11-hexadecen-1-ol were purchased from Pherobank and were 98% pure. Tetradecan-1-ol and methyl-nonadecanoate was purchased from Larodan with 99% purity.

### Qualitative Analysis:

The following compounds were identified based on their spectrum and retention time match with analytical standards: tetradecanal (14:Ald), pentadecanal (15:Ald), (Z)-9-hexadecenal (Z9-16:Ald), (Z)-11-hexadecenal (Z11-16:Ald), hexadecanal (16:Ald), and (Z)-11-hexadecen-1-ol (Z11-16:OH).

Monounsaturated pentadecenal (15-1:Ald) was identified based on its match with the spectrum in the NIST library.

### Preparation of Reaction Mixture

200 g fatty alcohol mixture comprising of 78% Z9-hexadecenol and Z11-hexadecenol was added to a 1 dm³ jacketed reaction vessel. 16 g 4 Å molecular sieves, 6.4 g of 2,2′-bipyridine, 3.6 g of 4-hydroxy-TEMPO, and 3.4 g of N-methylimidazole were added to said vessel. A solution of 15.6 g tetrakis acetonitrile copper (I) trifluoromethane sulfonate in 400 ml acetonitrile was added. 2 l/min air was bubbled through the solution for 2 h.

A representative embodiment of the crude reaction product produced using this method comprises about 30% fatty aldehyde, about 1.0% bipyridine, about 0.4% copper, about 0.6% 4-OH-TEMPO, about 0.5% 1-methylimidazole, and about 62% acetonitrile.

### Purification

100 g of the reaction mixture was removed and extracted with 165 g n-heptane. After vigorous stirring for 5 min the mixture was left for 30 min to allow for settling of the phases. The lower phase containing acetonitrile and the bulk of copper, bipyridine, N-methylimidazol and OH-TEMPO was discarded, the top phase containing mainly n-heptane and pheromone product was collected, and heptane evaporated at 65° C. 15 mbar. Yielding 32 g product containing 74% Z9-hexadecenol and Z11-hexadecenol.

### Example 9: Purification of Reaction Mixture

100 g of the reaction mixture produced in Example 8 was removed and extracted with 165 g n-heptane and 1 g glacial acetic acid. After vigorous stirring for 5 min the mixture was left for 30 min to allow for separation of the phases. The lower phase was discarded, the top phase was collected, and heptane evaporated at 65° C. to 15 mbar. Yielding 30 g product with 74.9% Z9-hexadecenol and Z11-hexadecenol.

### Example 10: Purification of Reaction Mixture

100 g of the reaction mixture produced in example 7 was removed and extracted with 165 g n-heptane and 1.2 g glacial acetic acid. After vigorous stirring for 5 min the mixture was left for 30 min to allow for separation of the phases. The lower phase was discarded, the top phase was collected, and heptane evaporated at 65° C. to 15 mbar. Yielding 30.2 g product with 73.8% Z9-hexadecenol and Z11-hexadecenol.

### Example 11: Purification of Reaction Mixture

100 g of the reaction mixture produced in example 7 was removed and extracted with 165 g n-heptane and 2 g glacial acetic acid. After vigorous stirring for 5 min the mixture was left for 30 min to allow for separation of the phases. The lower phase was discarded, the top phase was collected, and heptane evaporated at 65° C. to 15 mbar. Yielding 35.6 g product with 74.2% Z9-hexadecenol and Z11-hexadecenol.

### Example 12: Comparative Example of Typical Procedure for the Oxidation of Z11-16:OH (Z11-Hexadecenol) Oil and Aqueous Work Up

In a 500 ml bottle equipped with an air bubbler and a reflux condenser is added the starting material 100 g (86% total alcohol purity; 0.36 mol and 60.04% of the active pheromone Z11-16:OH (Z11-hexadecenol)) and 200 ml of CH₃CN. To the suspension are then added 2.87 g of copper (I) bromide (5 mol %; 0.02 mol), 2.79 g of 2,2′-bipyridine (Bipy) (5 mol %; 0.02 mol), 1.40 g of (4-hydroxy-2,2,6,6-tetramethylpiperidin-1-oxyl (4-OH-TEMPO) (2.5 mol %; 0.01 mol), 1.43 ml of N-methyl imidazole (5 mol %; 0.02 mol; 1.47 g). The reaction was stirred until the Z11-16:OH signal disappeared on GCMS (16/20 h).

Acetonitrile in the mixture was evaporated at reduced pressure. The residue was diluted with 200 ml of ethyl acetate and transferred in a separatory funnel. The solution was washed with 2×200 ml solution of 0.5 N H₂SO₄, or until the organic layer lost the blue coloration.

The solution was further washed with 1×100 ml sodium thiosulfate saturated solution and 1×50 ml of NaCl saturated solution. The combined organic fractions were dried over sodium sulfate, filtered, and then concentrated at reduced pressure.

### Example 13: Comparison of Purification Protocols and Product Stability

### Results

Table 7 shows a comparison of copper, oxidant, and ligand content in reaction products purified as outlined in examples 7 to 12.

TABLE 7
content of impurities in purified products from Examples 8 to 12.

| | Example 12 | Exam- | Exam- | Exam- | Exam- |
|---|---|---|---|---|---|
| Component | (Aqueous) | ple 8 | ple 9 | ple 10 | ple 11 |
| Non-accounted | 18% | 12% | 12% | 12% | 12% |
| for (wt %) | | | | | |
| Recovered oil: | | | | | |
| [Cu] adsorption | 0.7 | 0.76 | 0.035 | 0.034 | 0.02 |
| at 680 in 5 mm | | | | | |
| cuvette | | | | | |
| 4-OH-TEMPO | 1.5% | 0.60% | 0.61% | 0.62% | 0.66% |
| BIPY | <0.1% | <0.1% | <0.1% | <0.1% | <0.1% |

The methods of the present disclosure (Examples 8 to 11) provided purified products having much lower content of non-accounted for components (12%) than the products obtained using the comparative purification methodology of Example 12 (18%).

The copper content, which was assessed by the absorption at 680 nm in a 5 mm cuvette, were high for the comparative example 12, having an absorption of 0.7. Example 8, wherein no acid was added during purification, had a similar amount of copper as evidenced by the absorption of 0.76. Example 9, wherein 1 g acetic acid was added during purification, exhibited a much lower absorption 0.035, indicating a low content of copper. This trend continued for both Examples 10 and 11 wherein 1.5 and 2 g acetic acid was added, respectively. Specifically, the product of Example 11 showed the lowest absorption at 0.02. Based on these findings, it is contemplated that carboxylic acids and/or carboxylates are capable of coordinating to the copper ions, in turn facilitating their separation from the purified product.

The content of 4-OH-TEMPO and its reduced form in the product of comparative example 12 was relatively high at 1.5%. In contrast, the contents of 4-OH-TEMPO in the purified products of Examples 8 to 11 were relatively low at 0.60-0.66%, demonstration that the purification protocol is also efficient at removing this by product.

With regards to the ligand BIPY, the presently disclosed purification protocol provided contents below those quantifiable, which is a similar performance as the comparative protocol of Example 12.

### Example 14: Stability of Purified Products

Impurities present in the purified products have significant and negative impact on product stability. Table 8 shows the initial purity and the purity after 25 days.

TABLE 8
Product stability.

| | (Z)-hexadec-9-enal + (Z)- | | Total quantified fatty alcohols | |
|---|---|---|---|---|
| | hexadec-11-enal (wt %) | | and aldehydes* (wt %) | |
| Example | Fresh | After 25 Days | Fresh | After 25 Days |
| 8 | 75 | 56 | 89 | 72 |
| 9 | 75 | 64 | 89 | 82 |
| 10 | 74 | 69 | 88 | 87 |
| 11 | 74 | 68 | 88 | 86 |

*sum of tetradecanal, tetradecanoyl, pentadecenal, pentadecanal, pentadecen-1-ol, pentadecan-1-ol, (Z)-hexadec-9-enal, (Z)-hexadec-11-enal, hexadecanal, (Z)-hexadec-9-en-1-ol, (Z)-hexadec-11-en-1-ol, and hexadecan-1-ol.

Product stability improved significantly with reduced amounts of copper. The product of Example 8, which exhibited an absorption at 680 nm of 0.76, experienced a reduction in (Z)-hexadec-9-enal+ (Z)-hexadec-11-enal of approximate 25% after 25 days. In comparison, the purified products of Examples 9 to 11, which exhibited lower copper content (absorption 0.035 to 0.02 at 680 nm), experienced reductions in (Z)-hexadec-9-enal+ (Z)-hexadec-11-enal of only 15%, 7%, and 8%, respectively, after 25 days. A similar trend in stability was seen for the total quantified fatty alcohol and aldehyde content. Thus, the presently disclosed purification protocols provides for mores table compositions of fatty alcohols.

### Example 15: Preparation of Catalyst in a 1.5 m3 Reactor

23.6 kg Copper (II) trifluoromethanesulphonate was added to a 1500 L stainless steel vessel equipped with an anchor stirrer and a reflux condenser. 17.5 kg of copper shot (0.8-2.0 mm) and 12.2 kg copper granules (3+14 mesh) were added to the tank. 630 kg of acetonitrile was added. The mixture was stirred at 40 rpm and heated to 85-90° C. After around 3 hours of refluxing, the mixture was cooled down to room temperature.

The mixture was filtrated using a Guedu filter. The filtration speed was 60-70 liters/hour, yielding around 770 liters of catalyst containing liquid. In total 640 kg of catalyst solution was produced. It was divided over 2 transportable vessels containing approximately 320 kg each and used in Examples 16 and 17.

### Example 16: Oxidation of Fatty Alcohol Mixture at in 4 m3 Reactor

A fatty alcohol mixture consisting of predominantly of Z11-hexadecene-1-ol, Z9-hexadecene-1-ol and hexadecane-1-ol of the proportion listed in table 9.

TABLE 9

| Retention time/min | Compound | Avg., % w/w | STDEV |
|---|---|---|---|
| 11.4 | 1-Tetradecanol | <LOQ | |
| 14.5 | 1-Pentadecanol | <LOQ | |
| 16.1 | (Z)-9-hexadecan-1-ol | 4.6 | 0.2 |
| 16.2 | (Z)-11-hexadecan-1-ol | 82.0 | 3.0 |
| 16.4 | Hexadecan-1-ol | 8.9 | 0.3 |
| | Total | 95.4 | 3.48 |

A 4000 L reaction vessel equipped with a dissolved oxygen probe was charged with:

315 kg Biophero Z11-hexadecenol mixture
315 kg acetonitrile
10 kg 2,2-bipyridine
5.5 kg 4-hydroxyTEMPO
5.5 kg 1-methyl imidazole
25 kg 4 Å molecular sieves

The DO probe was calibrated by introducing air to the medium while agitating the vessel. Subsequently the 320 kg of catalyst solution in acetonitrile was transferred into the fermenter. At that moment, the reaction started. The settings of the reaction are shown in Table 10.

TABLE 10
oxidation reaction settings MOT2106

| Stirrer speed | 200 | rpm |
|---|---|---|
| Temperature | Controlled 30° C. | |
| Aeration | 93 | kg/h |

| | |
|---|---|
| Pressure | 0.5 barg (1.5 bara) |

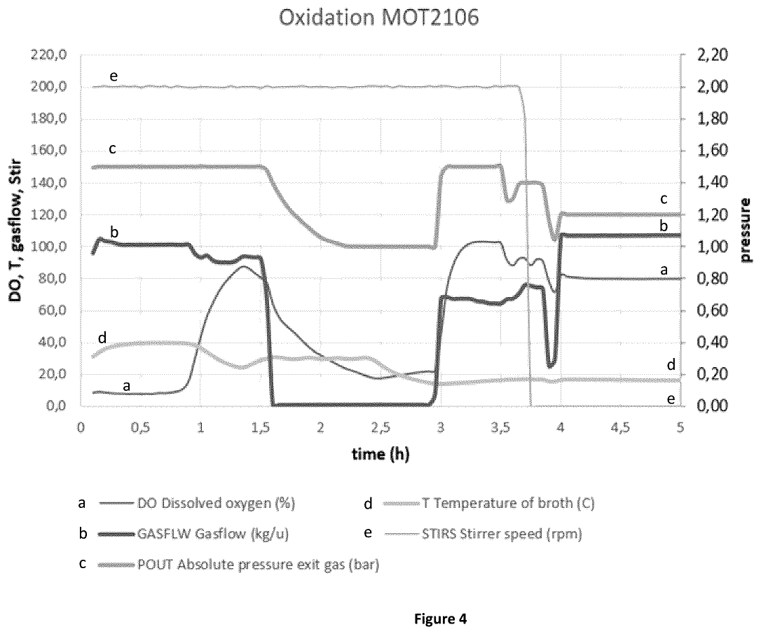

The oxidation reaction was closely monitored and sampled every 30 minutes. FIG. 4 shows the reaction data. At t=0 the catalyst addition was finished and the airflow was switched on. It can be seen that after 1 hour of reaction time the dissolved oxygen level is going up substantially, also the temperature decreased again. These things are indicating that the oxidation to the aldehyde form is complete. With the information from the DO and the GC analyses, it was decided to stop the airflow after 1.5 hours of reaction time.

Between 1.5 and 3 hours time, the contents of the reaction was in the waiting phase. From 1.5-2.5 hours the temperature was still maintained at 30° C. At 2.5 hours the temperature was held at around 15° C.

At 3 hours the vessel was emptied. This is done with air pressure on the head of the vessel, it does show up as air flow in the trend. At 4 hours the vessel was completely emptied into 2 IBCs. The mass of oxidation mixture is estimated at 980 kg.

After 1 hour, 94% conversion was reached. A final conversion of 99% was achieved. Over the waiting time (1.5 h—end sample) there is a small conversion increase from 97%-99%.

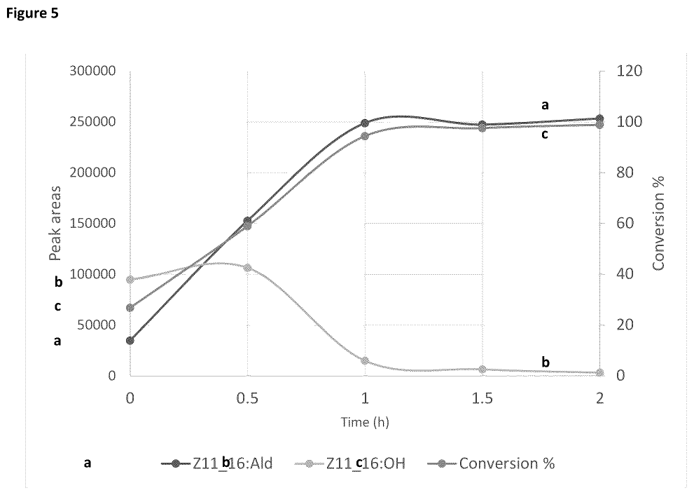

In Table 11 and FIG. 5 the Z11-hexadecenal conversion over time is shown.

TABLE 11

| | | | Total peak | Conversion |
|---|---|---|---|---|
| Time (h) | Z11_16:Ald | Z11_16:OH | area | % |
| 0 | 34617 | 94691 | 129308 | 27 |
| 0.5 | 152447 | 106319 | 258766 | 59 |
| 1 | 248650 | 14862 | 263512 | 94 |
| 1.5 | 247098 | 6364 | 253462 | 97 |
| End sample (2 h) | 253196 | 3136 | 256332 | 99 |

### Example 17: Oxidation of Fatty Alcohol Mixture at in 4 m³ Reactor

A 4000 L reaction vessel equipped with a dissolved oxygen sensor was charged with:

254 kg Biophero Z11-hexadecenol as in example 16
315 kg acetonitrile
10 kg 2,2-bipyridine
5.5 kg 4-hydroxyTEMPO
5.5 kg 1-methyl imidazole
25 kg 4 Å Molecular sieves

The oxidation reaction was performed in the 4000 L 40R10 fermenter. First the contents of the IBC with the reaction formulation was pressed into the fermenter. Next, 25 kg of molecular sieves were added to the vessel from the top. Then the DO probe was calibrated by introducing air to the medium while agitating the vessel.

The catalyst solution from example A was transferred into the fermenter agitation speed set 51

RPM and aeration started. The settings of the reaction are shown in Table 12.

TABLE 12

| Parameter | Setpoint | |
|---|---|---|
| Stirrer speed | 51 | rpm |
| Temperature | Controlled 30° C. | |
| Aeration | 93 | kg/h |

| | |
|---|---|
| Pressure | 0.5 barg (1.5 bara) |

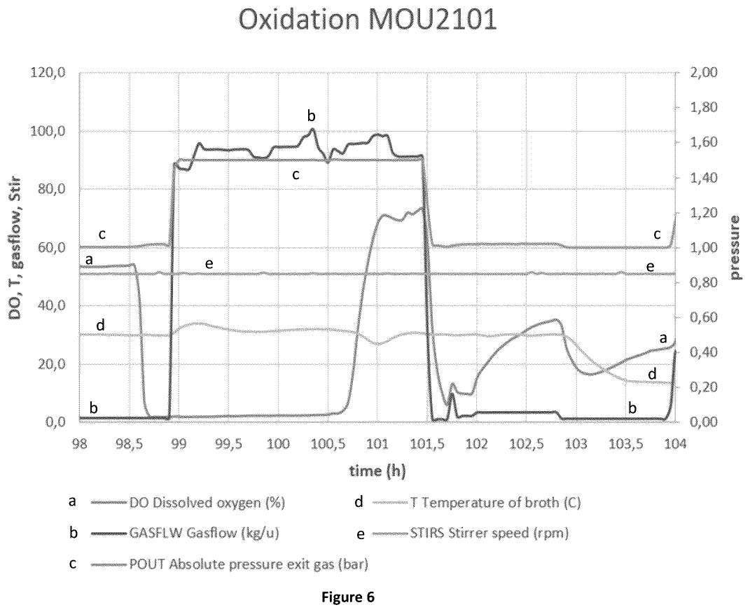

The oxidation reaction sampled every 30 minutes. FIG. 6 shows the online reaction data of the oxidation process. At t=98.5 the catalyst solution is introduced into the vessel. At around 99 h, the airflow is switched on and kept between 85 and 100 kg/h for 2.5 hours. Between 101.5 and 104 hours, the contents of the fermenter was in the waiting phase. During this phase there was a 3.5 kg/h airflow through the sparger.

A maximum temperature of around 34° C. was observed (between t=99 and 99.5 h. The liquid was cooled to around 15° C. at t=103 h.

TABLE 13
Z11-hexadecenal conversion over time during MOU2101

| | | | Total | |
|---|---|---|---|---|
| Time (h) | Z11_16:Ald | Z11_16:OH | peak area | Conversion % |
| 0 | 46645 | 150376 | 197021 | 24 |
| 0.5 | 53873 | 97395 | 151268 | 36 |
| 1 | 140358 | 102612 | 242970 | 58 |
| 1.5 | 200203 | 48008 | 248211 | 81 |
| 2 | 213234 | 7071 | 220305 | 97 |
| 2.5 | 218600 | 1917 | 220517 | 99 |

### Example 18: Oxidation of Z11,Z13-16:OH to Corresponding Aldehyde Z11,Z13-16:Ald

A mixture of primary fatty alcohols containing 73 wt % Z11,Z13-16:OH ((Z11,Z13)-hexadecadien-1-ol) in a mixture was used as a representative sample for conversion to aldehydes.

The Z11,Z13-16:OH mixture (8 g), 2,2′-bipyridine (0.25 g), 2,2,6,6-tetramethyl piperidinyl oxyl (0.14 g) and 1-methylimidazole (0.13 g) was added to acetonitrile (20 ml). To the aforementioned solution, tetrakisacetonitrilecopper (I) trifluoromethanesulfonate in 10 ml acetonitrile was added.

The temperature of the reaction mixture was controlled to 30° C. while air was sparged through the solution at a rate of 1 l/min. The air sparging was halted after 164 min and the reaction mixture was diluted in 1-heptane (40 ml) and the mixture extracted with water (20 ml). The top phase was evaporated to 10 mbar at 60° C. giving a product containing 65.5 wt % Z11,Z13-16:Ald ((Z11,Z13)-hexadecadienal) with a residual amount of 3.4 wt % Z11,Z13-16:OH.

| Start product: | End product |
|---|---|
| 73 wt % Z11, Z13-16:OH | 3.4 wt % Z11, Z13-16:OH |
| | 65.5 wt % Z11, Z13-16:Ald |

These data shows that the fatty alcohol Z11,Z13-16:OH was chemically oxidized to the corresponding aldehyde, Z11,Z13-16:Ald.

### Example 19-Comparative Example of Small Versus Large Scale Oxidation

### Small Scale 1

A mixture of fatty alcohols 24 g comprising of fatty alcohols in table 3 was oxidised in a shake flask open to air with above mentioned fatty alcohol mix and acetonitrile 5 g. To the reaction mixture a catalyst comprising of 1.88 g tetrakisacetonitrilecopper (I) trifluoromethane sulfonate, 0.78 g 2.2′-bipyridine, 0.43 g 4-hydroxy TEMPO, 0.41 N-imidazole and 5.4 g 4 Å molecular sieves were added. The reaction was left for 2 h during at 30° C. The conversion increased steadily to over 97% at 120 min finally reaching 100% at 180 min.

### Small Scale 2

A mixture of fatty alcohols 250 g comprising of fatty alcohols in table 3 was oxidised in a glass reactor equipped with a stirrer and an air sparger. To the fatty alcohol mixture diluted with 545 g acetonitrile were added 19 g tetrakisacetonitrilecopper (I) trifluoromethane sulfonate, 8 g 2.2′-bipyridine, 5.3 g 4-hydroxy TEMPO and 6.5 g N-imidazole. The reaction mixed at room temperature for all the all reaction time. The conversion increased steadily to over 58% at 120 min finally reaching 93% after 20 hours.

### Large Scale

Step 1: Catalyst Cu(ACN)₄OTf solution was prepared from Copper (II) trifluoro methane and metal Copper. 100 L acetonitrile were added to a stainless-steel vessel with anchor stirrer. Then, 2.81 kg Cu(Otf)₂ and 2.8 kg copper granulate were added under gentle stirring. The mixture was heated to 85° C. After 5.5 hours refluxing the mixture was cooled down to room temperature. Subsequently, the mixture was pressed through a filter to remove the remaining copper granulate. The process afforded 90 L of light yellowish solution used in the following step.

Step 2: The oxidation reaction was performed in the 300 L steel vessel with an air sparger. In the tank were added 60.8 kg Z11-Hexadecenol mixture, 50 L acetonitrile, 2.4 kg 2,2-bipyridine, 1.1 kg 4-hydroxyTEMPO and 1.3 kg, 1-methyl imidazole. Finally, the catalyst solution from step 1 was transferred to the oxidation vessel and the mixture was aerated under constant mixing with 10 Kg/h air. After 16 hours reaction time a conversion of 67% was reached.

### CONCLUSION

The present comparative example demonstrates that traditional oxidation methodology developed for small scale oxidation is not always applicable on large scale, such as kilogram scale. On larger scales, such as for commercial chemical production, high conversion and a clean reaction profile are critical process parameters, and the methods of the present disclosure provides a tangible solution to these needs in industry.

### REFERENCES

Stahl et al. J. Am. Chem. Soc. 2011, 133, 16901-16910
Kumpulainen and Koskinen, Chem. Eur. J. 2009, 15, 10901-10911

### ITEMS

Further described herein are the following itemized embodiments:
1. A method of converting a fatty alcohol to a fatty aldehyde, said method comprising the steps of:
   a) providing a reaction mixture comprising a fatty alcohol, a catalyst comprising a copper source, and a solvent, and
   b) oxidizing the fatty alcohol by adding O₂ to the reaction mixture in an amount sufficient for converting more than 50 wt % of the fatty alcohol to fatty aldehyde and less than 50 wt % into fatty acid.
2. The method of item 1 comprising adding at least 0.049 μmol dissolved O₂ per minute per μmol copper in the reaction mixture.
3. The method of any preceding item, comprising adding at least 0.0025 μmol dissolved O₂ per minute per μmol initial fatty alcohol in the reaction mixture.
4. The method of any preceding item, comprising adding at least 0.025 μmol dissolved O₂ per minute per μmol fatty acid in the reaction mixture.
5. The method of any preceding item comprising adding at least 10 μmol O₂, such as at least 20 μmol O₂, at least 40 μmol O₂, or at least 60 μmol O₂ per minute per gram of fatty alcohol to the reaction mixture, thereby obtaining the fatty aldehyde, optionally wherein the fatty alcohol and the fatty aldehyde are desaturated.
   6. The method of any preceding item, wherein the Oz is added by mixing the reaction mixture with a gas or a liquid comprising O₂, optionally enriched with 02.
   7. The method of any preceding item, wherein the mixing is made by bubbling a gas mixture comprising O₂ through the reaction mixture.
   8. The method of item 1, wherein the conversion is the conversion of a primary alcohol functional group to an aldehyde functional group.
   9. The method of any preceding items, wherein the conversion is oxidation of a primary alcohol functional group to an aldehyde functional group.
   10. The method of any preceding items wherein the fatty alcohol is a primary alcohol.
   11. The method of any preceding items, wherein the fatty alcohol is a saturated fatty alcohol.
   12. The method of any preceding items, wherein the fatty alcohol is a desaturated fatty alcohol.
   13. The method of any preceding items, wherein the fatty alcohol is a C10 to C26 fatty alcohol.
   14. The method of any preceding items, wherein the fatty alcohol is a C10 to C22 fatty alcohol.
   15. The method of any preceding items, wherein the fatty alcohol is a C12 to C20 fatty alcohol.
   16. The method of any preceding items, wherein the fatty alcohol is a C12 to C18 fatty alcohol.
   17. The method of any preceding items, wherein the fatty alcohol is a C12, C14, C16 or C18 fatty alcohol.
   18. The method of any preceding items, wherein the desaturated fatty alcohol has a double bond at position 9, 11 or 13, or wherein the desaturated fatty alcohol has double bonds at positions 9 and 11, or at positions 11 and 13.
   19. The method of any preceding items, wherein the desaturated fatty alcohol has a double bond at position 9 or 12, or wherein the desaturated fatty alcohol has double bonds at positions 9 and 12.
   20. The method of any preceding items, wherein the desaturated fatty alcohol has a double bond at position 8 or 10, or wherein the desaturated fatty alcohol has double bonds at positions 8 and 10.
   21. The method of any preceding items, wherein the fatty alcohol has a carbon chain length of 12, 14, or 16.
   22. The method of any preceding items, wherein the fatty alcohol is an unbranched fatty alcohol.
   23. The method of any preceding items, wherein the fatty alcohol is selected from the group consisting of:
   (Z)-Δ3 desaturated fatty alcohols having a carbon chain length of 8, 9, 10, 11, 12, 13, 14, 15, 16, 17, 18, 19, 20, 21 or 22;
   (E)-Δ3 desaturated fatty alcohols having a carbon chain length of 8, 9, 10, 11, 12, 13, 14, 15, 16, 17, 18, 19, 20, 21 or 22;
   (Z)-Δ5 desaturated fatty alcohols having a carbon chain length of 8, 9, 10, 11, 12, 13, 14, 15, 16, 17, 18, 19, 20, 21 or 22;
   (E)-Δ5 desaturated fatty alcohols having a carbon chain length of 8, 9, 10, 11, 12, 13, 14, 15, 16, 17, 18, 19, 20, 21 or 22;
   (Z)-Δ6 desaturated fatty alcohols having a carbon chain length of 8, 9, 10, 11, 12, 13, 14, 15, 16, 17, 18, 19, 20, 21 or 22;
   (E)-Δ6 desaturated fatty alcohols having a carbon chain length of 8, 9, 10, 11, 12, 13, 14, 15, 16, 17, 18, 19, 20, 21 or 22;
   (Z)-Δ7 desaturated fatty alcohols having a carbon chain length of 8, 9, 10, 11, 12, 13, 14, 15, 16, 17, 18, 19, 20, 21 or 22; (E)-Δ7 desaturated fatty alcohols having a carbon chain length of 8, 9, 10, 11, 12, 13, 14, 15, 16, 17, 18, 19, 20, 21 or 22;
   (Z)-Δ8 desaturated fatty alcohols having a carbon chain length of 9, 10, 11, 12, 13, 14, 15, 16, 17, 18, 19, 20, 21 or 22;
   (E)-Δ8 desaturated fatty alcohols having a carbon chain length of 9, 10, 11, 12, 13, 14, 15, 16, 17, 18, 19, 20, 21 or 22;
   (Z)-Δ9 desaturated fatty alcohols having a carbon chain length of 10, 11, 12, 13, 14, 15, 16, 17, 18, 19, 20, 21 or 22;
   (E)-Δ9 desaturated fatty alcohols having a carbon chain length of 10, 11, 12, 13, 14, 15, 16, 17, 18, 19, 20, 21 or 22;
   (Z)-Δ10 desaturated fatty alcohols having a carbon chain length of 11, 12, 13, 14, 15, 16, 17, 18, 19, 20, 21 or 22;
   (E)-Δ10 desaturated fatty alcohols having a carbon chain length of 11, 12, 13, 14, 15, 16, 17, 18, 19, 20, 21 or 22;
   (Z)-Δ11 desaturated fatty alcohols having a carbon chain length of 12, 13, 14, 15, 16, 17, 18, 19, 20, 21 or 22;
   (E)-Δ11 desaturated fatty alcohols having a carbon chain length of 12, 13, 14, 15, 16, 17, 18, 19, 20, 21 or 22;
   (Z)-Δ12 desaturated fatty alcohols having a carbon chain length of 13, 14, 15, 16, 17, 18, 19, 20, 21 or 22; (E)-Δ12 desaturated fatty alcohols having a carbon chain length of 13, 14, 15, 16, 17, 18, 19, 20, 21 or 22;
   (Z)-Δ13 desaturated fatty alcohols having a carbon chain length of 14, 15, 16, 17, 18, 19, 20, 21 or 22; and (E)-Δ13 desaturated fatty alcohols having a carbon chain length of 14, 15, 16, 17, 18, 19, 20, 21 or 22.
   24. The method of any preceding items, wherein the fatty alcohol is selected from the group consisting of (E)7,(Z)9 desaturated fatty alcohols having a carbon chain length of 10, 11, 12, 13, 14, 15, 16, 17, 18, 19, 20, 21, or 22,
   (E)3, (Z)8, (Z)11 desaturated fatty alcohols having a carbon chain length of 12, 13, 14, 15, 16, 17, 18, 19, 20, 21, or 22,
   (Z)9, (E)11, (E)13 desaturated fatty alcohols having a carbon chain length of 14, 15, 16, 17, 18, 19, 20, 21, or 22,
   (Z)11, (Z)13 desaturated fatty alcohols having a carbon chain length of 14, 15, 16, 17, 18, 19, 20, 21 or 22, (Z)9, (E)12 desaturated fatty alcohols having a carbon chain length of 13, 14, 15, 16, 17, 18, 19, 20, 21 or 22,
   (E)7, (E)9 desaturated fatty alcohols having a carbon chain length of 10, 11, 12, 13, 14, 15, 16, 17, 18, 19, 20, 21 or 22, and
   (E8,E10) desaturated fatty alcohols having a carbon chain length of 11, 12, 13, 14, 15, 16, 17, 18, 19, 20, 21, or 22.
   25. The method of any preceding items, wherein the fatty alcohol is selected from the group consisting of:
   (E)7,(Z)9 desaturated fatty alcohol having a carbon chain length of 14,
   (E)3,(Z)8,(Z)11 desaturated fatty alcohol having a carbon chain length of 14,
   (Z)9,(E)11,(E)13 desaturated fatty alcohol having a carbon chain length of 14,
   (E)7,(Z)9 desaturated fatty alcohol having a carbon chain length of 12,
   (E)3,(Z)8,(Z)11 desaturated fatty alcohol having a carbon chain length of 12,
   (Z)9,(E)11,(E)13 desaturated fatty alcohol having a carbon chain length of 12,
   (E)8,(E)10 desaturated fatty alcohol having a carbon chain length of 12,
   (E)7,(E)9 desaturated fatty alcohol having a carbon chain length of 11,
   (Z)11,(Z)13 desaturated fatty alcohol having a carbon chain length of 16, and
   (Z)9,(E)12 desaturated fatty alcohol having a carbon chain length of 14.
   26. The method of any preceding items, wherein the fatty alcohol is selected from the group consisting of tetradecan-1-ol, pentadecan-1-ol, hexadecan-1-ol, pentadecen-1-ol, (Z)-9-hexadecen-1-ol, (Z)-11-hexadecen-1-ol, (7E,9E)-undeca-7,9-dien-1-ol, (11Z, 13Z)-hexadecadien-1-ol, (9Z, 12E)-tetradecadien-1-ol, and (8E,10E)-dodecadien-1-ol.
   27. The method of any preceding items, wherein the fatty alcohol composition comprises at least 30 wt % of one or more fatty alcohols, such as at least 40 wt %, 50 wt %, 55 wt %, such as 60 wt % of one or more fatty alcohols.
   28. The method of any preceding items, wherein the obtained fatty aldehyde is obtained as a fatty aldehyde composition comprising at least 30 wt % of one or more fatty aldehydes, such as at least 40 wt %, 50 wt %, 55 wt %, such as 60 wt % of one or more fatty aldehydes.
   29. The method of any preceding items, wherein the fatty aldehyde is a saturated fatty aldehyde.
   30. The method of any preceding items, wherein the fatty aldehyde is a desaturated fatty aldehyde.
   31. The method of any preceding items, wherein the fatty aldehyde is a C10 to C26 fatty aldehyde.
   32. The method of any preceding items, wherein the fatty aldehyde is a C10 to C22 fatty aldehyde.
   33. The method of any preceding items, wherein the fatty aldehyde is a C12 to C20 fatty aldehyde.
   34. The method of any preceding items, wherein the fatty aldehyde is a C12, C14, or C16 fatty aldehyde.
   35. The method of any preceding items, wherein the fatty aldehyde is an unbranched fatty aldehyde.
   36. The method of any preceding items, wherein the desaturated fatty aldehyde has a double bond at position 9, 11 or 13, or wherein the desaturated fatty aldehyde has double bonds at positions 9 and 11, or at positions 11 and 13.
   37. The method of any preceding items, wherein the desaturated fatty aldehyde has a double bond at position 9 or 12, or wherein the desaturated fatty aldehyde has double bonds at positions 9 and 12.
   38. The method of any preceding items, wherein the desaturated fatty aldehyde has a double bond at position 8 or 10, or wherein the desaturated fatty aldehyde has double bonds at positions 8 and 10.
   39. The method according to any one of the preceding items, wherein the fatty aldehyde has a carbon chain length of 12, 14, or 16.
   40. The method of any preceding items, wherein the fatty aldehyde is selected from the group consisting of:
   (Z)-Δ3 desaturated fatty aldehydes having a carbon chain length of 8, 9, 10, 11, 12, 13, 14, 15, 16, 17, 18, 19, 20, 21 or 22;
   (E)-Δ3 desaturated fatty aldehydes having a carbon chain length of 8, 9, 10, 11, 12, 13, 14, 15, 16, 17, 18, 19, 20, 21 or 22;
   (Z)-Δ5 desaturated fatty aldehydes having a carbon chain length of 8, 9, 10, 11, 12, 13, 14, 15, 16, 17, 18, 19, 20, 21 or 22;
   (E)-Δ5 desaturated fatty aldehydes having a carbon chain length of 8, 9, 10, 11, 12, 13, 14, 15, 16, 17, 18, 19, 20, 21 or 22;
   (Z)-Δ6 desaturated fatty aldehydes having a carbon chain length of 8, 9, 10, 11, 12, 13, 14, 15, 16, 17, 18, 19, 20, 21 or 22;
   (E)-Δ6 desaturated fatty aldehydes having a carbon chain length of 8, 9, 10, 11, 12, 13, 14, 15, 16, 17, 18, 19, 20, 21 or 22;
   (Z)-Δ7 desaturated fatty aldehydes having a carbon chain length of 8, 9, 10, 11, 12, 13, 14, 15, 16, 17, 18, 19, 20, 21 or 22;
   (E)-Δ7 desaturated fatty aldehydes having a carbon chain length of 8, 9, 10, 11, 12, 13, 14, 15, 16, 17, 18, 19, 20, 21 or 22;
   (Z)-Δ8 desaturated fatty aldehydes having a carbon chain length of 9, 10, 11, 12, 13, 14, 15, 16, 17, 18, 19, 20, 21 or 22;
   (E)-Δ8 desaturated fatty aldehydes having a carbon chain length of 9, 10, 11, 12, 13, 14, 15, 16, 17, 18, 19, 20, 21 or 22;
   (Z)-Δ9 desaturated fatty aldehydes having a carbon chain length of 10, 11, 12, 13, 14, 15, 16, 17, 18, 19, 20, 21 or 22;
   (E)-Δ9 desaturated fatty aldehydes having a carbon chain length of 10, 11, 12, 13, 14, 15, 16, 17, 18, 19, 20, 21 or 22;
   (Z)-Δ10 desaturated fatty aldehydes having a carbon chain length of 11, 12, 13, 14, 15, 16, 17, 18, 19, 20, 21 or 22;
   (E)-Δ10 desaturated fatty aldehydes having a carbon chain length of 11, 12, 13, 14, 15, 16, 17, 18, 19, 20, 21 or 22;
   (Z)-Δ11 desaturated fatty aldehydes having a carbon chain length of 12, 13, 14, 15, 16, 17, 18, 19, 20, 21 or 22;
   (E)-Δ11 desaturated fatty aldehydes having a carbon chain length of 12, 13, 14, 15, 16, 17, 18, 19, 20, 21 or 22;
   (Z)-Δ12 desaturated fatty aldehydes having a carbon chain length of 13, 14, 15, 16, 17, 18, 19, 20, 21 or 22;
   (E)-Δ12 desaturated fatty aldehydes having a carbon chain length of 13, 14, 15, 16, 17, 18, 19, 20, 21 or 22;
   (Z)-Δ13 desaturated fatty aldehydes having a carbon chain length of 14, 15, 16, 17, 18, 19, 20, 21 or 22; and
   (E)-Δ13 desaturated fatty aldehydes having a carbon chain length of 14, 15, 16, 17, 18, 19, 20, 21 or 22.
   41. The method of any preceding items, wherein the fatty aldehyde is selected from the group consisting of
   (E)7,(Z)9 desaturated fatty aldehydes having a carbon chain length of 10, 11, 12, 13, 14, 15, 16, 17, 18, 19, 20, 21, or 22,
   (E)3,(Z)8,(Z)11 desaturated fatty aldehydes having a carbon chain length of 12, 13, 14, 15, 16, 17, 18, 19, 20, 21, or 22,
   (Z)9,(E)11,(E)13 desaturated fatty aldehydes having a carbon chain length of 14, 15, 16, 17, 18, 19, 20, 21, or 22,
   (Z)11,(Z)13 desaturated fatty aldehydes having a carbon chain length of 14, 15, 16, 17, 18, 19, 20, 21 or 22,
   (Z)9,(E)12 desaturated fatty aldehydes having a carbon chain length of 13, 14, 15, 16, 17, 18, 19, 20, 21 or 22,
   (E)7,(E)9 desaturated fatty aldehydes having a carbon chain length of 10, 11, 12, 13, 14, 15, 16, 17, 18, 19, 20, 21 or 22, and
   (E)8,(E)10 desaturated fatty aldehydes having a carbon chain length of 11, 12, 13, 14, 15, 16, 17, 18, 19, 20, 21, or 22.
   42. The method of any preceding items, wherein the fatty aldehyde is selected from the group consisting of:
   (E)7,(Z)9 desaturated fatty aldehyde having a carbon chain length of 14,
   (E)3,(Z)8,(Z)11 desaturated fatty aldehyde having a carbon chain length of 14,
   (Z)9,(E)11,(E)13 desaturated fatty aldehyde having a carbon chain length of 14,
   (E)7,(Z)9 desaturated fatty aldehyde having a carbon chain length of 12,
   (E)3,(Z)8,(Z)11 desaturated fatty aldehyde having a carbon chain length of 12,
   (Z)9,(E)11,(E)13 desaturated fatty aldehyde having a carbon chain length of 12,
   (E)8,(E)10 desaturated fatty aldehyde having a carbon chain length of 12
   (E)7,(E)9 desaturated fatty aldehyde having a carbon chain length of 11,
   (Z)11,(Z)13 desaturated fatty aldehyde having a carbon chain length of 16, and
   (Z)9,(E)12 desaturated fatty aldehyde having a carbon chain length of 14.
   43. The method of any preceding items, wherein the fatty aldehyde is selected from the group consisting of tetradecan-1-al, pentadecan-1-al, hexadecan-1-al, pentadecen-1-al, (Z)-9-hexadecen-1-al, (Z)-11-hexadecen-1-al, (7E,9E)-undeca-7,9-dien-1-al, (11Z, 13Z)-hexadecadien-1-al, (9Z,12E)-tetradecadien-1-al, and (8E,10E)-dodecadien-1-al.
   44. The method of any preceding items, wherein the copper (I) source is a copper (I) salt.
   45. The method of any preceding items, wherein the copper (I) source is selected from the group consisting of tetrakisacetonitrile copper (I) tetrakisacetonitrile copper (I) tetrafluoroborate, triflate, tetrakisacetonitrile copper (I) hexafluorophosphate, and tetrakisacetonitrile copper (I) halide, and tetrakisacetonitrile copper (I) perchlorate.
   46. The method of any preceding items, wherein the copper (I) source comprises a copper (II) compound and a reductant.
   47. The method of any preceding items, wherein the copper (II) compound is a copper (II) salt.
   48. The method of any preceding items, wherein the copper (II) salt is selected from the group consisting of copper (II) triflate, copper (II) tetrafluoroborate, copper (II) hexafluorophosphate, copper (II) bromide, copper (II) chloride, copper (II) iodide, and copper (II) perchlorate.
   49. The method of any preceding items, wherein the reductant is selected from the group consisting of copper metal, zinc metal, aluminium metal, sodium hydrogensulfite, formic acid, salts of formic acid, oxalic acid, and salts of oxalic acid.
   50. The method of any preceding items, wherein the copper (I) source is a copper (II) salt and copper metal.
   51. The method of any preceding items, wherein the catalyst composition comprises a ligand.
   52. The method of any preceding items, wherein the ligand coordinates to copper (I) via nitrogen.
   53. The method of any preceding items, wherein the ligand comprises a pyridine moiety.
   54. The method of any preceding items, wherein the ligand is a bidentate nitrogen ligand.
   55. The method of any preceding items, wherein the ligand comprises a 2,2′-bipyridine moiety or a 2,2′-bipyrimidine moiety.
   56. The method of any preceding items, wherein the ligand is selected from the group consisting of 2,2′-bipyridine, 4,4′-dimethyl-2,2′-bipyridine, 5,5′-dimethyl-2,2′-bipyridine 2,2′-bipyrimidine, 2,2′-bipyridine-4,4′-dicarboxylic acid or an ester thereof, 2,2′-bipyridine-5,5′-dicarboxylic acid or an ester thereof.
   57. The method of any preceding items, wherein the catalyst composition comprises an aminoxyl radical compound.
   58. The method of any preceding items, wherein the aminoxyl radical compound is selected from the group consisting of TEMPO, (4-hydroxy-2,2,6,6-tetramethylpiperidin-1-yl)oxyl (4-OH-TEMPO), 4-acetamido-TEMPO, 4-hydroxy-TEMPO benzoate, 4-amino-TEMPO, 2-azaadamantane-N-oxyl, 9-2-azaadamantane-N-oxyl, 4-oxo-TEMPO, and a polymer functionalised with any of said aminoxyl radical compounds.
   59. The method of any preceding items, wherein the aminoxyl radical compound is (2,2,6,6-Tetramethylpiperidin-1-yl)oxyl (TEMPO) or a TEMPO derivative.
   60. The method of any preceding items, wherein the catalyst composition comprises a base.
   61. The method of any preceding items, wherein the base is an organic base.
   62. The method of any preceding items, wherein the base is selected from the group consisting of 1-methylimidazole, 1,8-diazabicyclo[5.4.0]undec-7-ene, 1,5-diazabicyclo[4.3.0]non-5-ene, 1,5,7-triazabicyclo[4.4.0]dec-5-ene, 1,1,3,3-tetramethylguanidine, 7-methyl-1,5,7-triazabicyclo-[4.4.0]-dec-5-ene, and potassium t-butoxide.
   63. The method of any preceding items, wherein exposition of the reaction mixture to 02 is performed at to 80° C., such as 10 to 70° C., such as 15 to 65° C.
   64. The method of any preceding items, wherein exposition of the reaction mixture to 02 is performed at a pressure of 0.5 to 40 bar, such as 0.5 to 30 bar, such as 0.6 to 20 bar, such as 0.7 to 10 bar, such as 0.8 to 5 bar.
   65. The method of any preceding items, wherein exposition of the reaction mixture to 02 is performed at a pressure of 0.8 to 1.2 bar.
   66. The method of any preceding items, wherein exposition of the reaction mixture to 02 is performed at ambient pressure.
   67. The method of any preceding items, wherein the reaction mixture is exposed to at least 0.3 ml 02 per minute per gram of fatty alcohol composition, such as at least 0.4 ml, 0.5 ml, 0.6 ml, 0.7 ml, 0.8 ml, 0.9 ml, 1.0 ml, 1.1 ml, 1.2 ml, 1.3 ml, 1.4 ml, such as at least 1.5 ml 02 per minute per gram of fatty alcohol composition, as assessed at a pressure of 1 bar.
   68. The method of any preceding items, wherein the reaction mixture is exposed to at least 0.3 ml 02 per minute per gram of fatty alcohol, such as at least 0.4 ml, 0.5 ml, 0.6 ml, 0.7 ml, 0.8 ml, 0.9 ml, 1.0 ml, 1.1 ml, 1.2 ml, 1.3 ml, 1.4 ml, such as at least 1.5 ml 02 per minute per gram of fatty alcohol, as assessed at a pressure of 1 bar.
   69. The method of any preceding items, wherein the reaction mixture is exposed to at least 60 ml 02 per minute per mol of fatty alcohol, such as at least 100 ml, 150 ml, 200 ml, 250 ml, 300 ml, 350 ml, 400 ml, such as at least 450 ml 02 per minute per mol of fatty alcohol, as assessed at a pressure of 1 bar.
   70. The method of any preceding items, wherein the reaction mixture is exposed to at least 10 μmol 02 per minute per gram of fatty alcohol, such as at least 12 μmol, 16 μmol, 20 μmol, 24 μmol, 28 μmol, 32 μmol, 36 μmol, 40 μmol, 44 μmol, 48 μmol, 52 μmol, 56 μmol, 60 μmol 02 per minute per gram of fatty alcohol.
   71. The method of any preceding items, wherein the reaction mixture is exposed to at least 2.5 mmol 02 per minute per mol of fatty alcohol, such as at least 4 mmol, 6 mmol, 8 mmol, 10 mmol, 12 mmol, 14 mmol, 16 mmol, such as at least 18 mmol 02 per minute per mol of fatty alcohol.
   72. The method of any preceding items, wherein the gas mixture comprises 5 to 100% O₂.
   73. The method of any preceding items, wherein the gas mixture comprises 15 to 25% O₂.
   74. The method of any preceding items, wherein the gas mixture comprises at least 90% O₂.
   75. The method of any preceding items, wherein the solvent is an aprotic, polar solvent.
   76. The method of any preceding items, wherein the solvent is selected from the group consisting of acetonitrile, dimethylformamide, acetonitrile, propionitrile, butyronitrile, dimethyl sulfoxide, dimethyl acetamide, and propylene carbonate.
   77. The method of any preceding items, wherein the amount of solvent corresponds to 0 to 2000% the weight of the fatty alcohol composition, such as 100 to 2000%, such as 100 to 1500%, such as 100 to 1000%, such as 100 to 500%.
   78. The method of any preceding items, wherein the amount of solvent corresponds to 100 to 2000% the weight of the fatty alcohol, such as 100 to 1500%, such as 100 to 1000%, such as 100 to 500%.
   79. The method of any preceding items, wherein the exposure to 02 is maintained for at least 5 minutes, such as at least 10 minutes, such as at least 20 minutes, such as at least 30 minutes, such as at least 40 minutes, such as at least 50 minutes, such as at least 60 minutes, such as at least 70 minutes, 80 minutes, 90 minutes, such as at least 100 minutes.
   80. The method of any preceding items, wherein the exposure to O₂ is carried out in a bubble column reactor or in a trickle bed reactor.
   81. The method of any preceding items, wherein the conversion of fatty alcohol is at least at least 60 wt %, such as at least 80 wt %, such as at least 85 wt %, 87 wt %, such as at least 90 wt %, such as at least wt 95 wt %, such as at least 99 wt %.
   82. The method of any preceding items, wherein the ratio of fatty acid produced to fatty aldehyde produced is less than 10:90.
   83. The method of any preceding item, wherein the conversion of fatty alcohol to fatty acid is les then 40 wt %, such as less than 30 wt %, such as less than 20 wt %, such as less than 15 wt %, such as less than 10 wt %, such as less than 5 wt %, such as less than 1 wt %.
   84. The method of any preceding items, further comprising removal of water from the reaction mixture.
   85. The method of any preceding items, wherein the removal of water from the reaction mixture is effected substantially throughout the exposure to O₂.
   86. The method of any preceding items, wherein the removal of water from the reaction mixture is effected using an adsorbent material.
   87. The method of any preceding items, wherein the adsorbent material is selected from the group consisting of molecular sieves, silica gel, alumina, bentonite clay, calcium oxide, an alkali metal carbonate, hydrogen carbonate, or an alkali earth metal carbonate.
   88. The method of any preceding item, further comprising a step of removing water from the reaction medium before, during or after the oxidation of the fatty alcohol.
   89. The method of item 88, further comprising adding a water absorbing or adsorbing material to the reaction medium absorbing or adsorbing water, optionally selected from molecular sieves, silica gels, aluminas, bentonite clays, calcium oxides, an alkali metal carbonates, hydrogen carbonates, or alkali earth metal carbonates or a combination thereof.
   90. The method of item 88 to 89, wherein the water absorbing or adsorbing material is added to the reaction medium in amounts, so that the water content in the reaction medium after the oxidation process is 2% by weight or less, and optionally the molar conversion of fatty alcohol to fatty aldehydes is more than 93%.
   91. The method of item 88 to 90, wherein the amount of water absorbing or adsorbing material added is at least 10 g per mmol of fatty alcohol present in the reaction medium prior to oxidation, such as at least 15 g per mmol of fatty alcohol, such as at least 19 g per mmol of fatty alcohol, and wherein optionally the water absorbing or adsorbing material is a molecular sieve.
   92. The method of any preceding items, where said method further comprises an initial step of producing the fatty alcohol, preferably wherein the fatty alcohol is desaturated, said initial step comprising the steps of:
   i. providing a yeast cell capable of producing the fatty alcohol, and
   ii. incubating said yeast cell in a medium,
   thereby producing the fatty alcohol.
   93. The method of any preceding items, wherein said method further comprises an initial step of producing the fatty alcohol, said initial step comprising the steps of:
      i. providing a yeast cell capable of synthesising alkanoyl-CoA, said yeast cell further capable of expressing:
         a desaturase, and
         an alcohol-forming fatty acyl-CoA reductase,
      ii. expressing said desaturase and said alcohol-forming fatty acyl-CoA reductase from said yeast cell, and
      iii. incubating said yeast cell in a medium, whereby the desaturase is capable of converting at least part of said alkanoyl-CoA to alkenoyl-CoA, and whereby said alcohol-forming fatty acyl-CoA reductase
      is capable of converting at least part of said alkenoyl-CoA to fatty alcohol,
   thereby producing said fatty alcohol.
   94. The method of any preceding items, wherein the fatty alcohol is (Z)-11-hexadecen-1-ol, wherein the alkanoyl-CoA is hexadecanoyl-CoA, wherein the desaturase is a A11-desaturase, and wherein the alkenoyl-CoA is (Z)-11-hexadecenoyl-CoA.
   95. The method of any preceding items, wherein said method further comprises an initial step of producing the fatty alcohol, said initial step comprising the steps of:
      i. providing an oleaginous yeast cell capable of producing a desaturated fatty alcohol, said yeast cell further:
         capable of expressing at least one heterologous fatty acyl-CoA desaturase capable of introducing at least one double bond in a fatty acyl-CoA, thus forming a desaturated fatty acyl-CoA,
         capable of expressing at least one heterologous fatty acyl-CoA reductase capable of converting at least part of said desaturated fatty acyl-CoA to a desaturated fatty alcohol,
         having a mutation resulting in reduced activity of a fatty alcohol oxidase and having a mutation resulting in reduced activity of at least one of: a fatty aldehyde dehydrogenase, a peroxisome biogenesis factor, and glycerol-3-phosphate acyltransferase, and
      ii. incubating said yeast cell in a medium,
   thereby producing the fatty alcohol.
   96. The method according to one of the preceding items, wherein said method further comprises an initial step of producing the fatty alcohol, said initial step comprising the steps of:
      i. providing a yeast cell capable of producing a desaturated fatty alcohol, said yeast cell expressing:
         at least one heterologous fatty acyl-CoA desaturase capable of introducing at least one double bond in a fatty acyl-CoA having a carbon chain length of 14, wherein said desaturase is selected from the group consisting of a 49 desaturase and a A11 desaturase, wherein the desaturase has a higher specificity towards tetradecanoyl-CoA then towards hexadecanoyl-CoA, and
      at least one heterologous fatty acyl-CoA reductase capable of converting at least part of said desaturated fatty acyl-CoA to a desaturated fatty alcohol, and
      ii. incubating said yeast cell in a medium,
   thereby producing the fatty alcohol.
   97. The method of any preceding items, wherein the method further comprises an initial step of producing the fatty alcohol, said initial step comprising providing a yeast cell capable of producing the fatty alcohol and culturing said yeast cell in a culture medium under conditions allowing production of said fatty alcohol, wherein the culturing medium comprises an extractant in an amount equal to or greater than its cloud concentration measured in an aqueous solution such as the culture medium at the cultivation temperature, wherein the extractant is a non-ionic ethoxylated surfactant, thereby producing the fatty alcohol.
   98. The method of any preceding items, wherein the method further comprises an initial step of producing the fatty alcohol, preferably wherein the fatty alcohol is a desaturated fatty alcohol, said initial step comprising
      i. providing a yeast cell capable of producing a fatty alcohol ester, and culturing said yeast cell in a culture medium under conditions allowing production of said fatty alcohol ester, wherein the culturing medium comprises an extractant in an amount equal to or greater than its cloud concentration measured in an aqueous solution such as the culture medium at the cultivation temperature, wherein the extractant is a non-ionic ethoxylated surfactant, thereby producing the fatty alcohol ester, and
      ii. converting said fatty alcohol ester to the fatty alcohol,
   thereby producing the fatty alcohol.
   99. A fatty aldehyde purification method comprising the steps of:
      a) providing a crude reaction product comprising:
         i. a fatty aldehyde,
         ii. copper ions, and
         iii. a polar solvent;
      b) mixing said crude reaction product with an apolar, aprotic solvent and an acid to create an apolar phase and a polar phase; and
      c) separating the apolar phase from the polar phase.
   100. The fatty aldehyde purification method of any preceding items, wherein the crude reaction product comprises:
      i. 5 to 80% of the fatty aldehyde,
      ii. 0.05 to 5.0% copper ions,
      iii. 20 to 95% of the polar solvent.
   101. The fatty aldehyde purification method of any preceding items, wherein the crude reaction product comprises 0.05 to 5.0% copper ions, such as 0.05 to 2.0% copper ions, such as 0.05 to 1.0% copper ions.
   102. The fatty aldehyde purification method of any preceding items, wherein the crude reaction product further comprises a ligand, such as 0.1 to 10% of a ligand, such as 0.1 to 5% of a ligand, such as 0.1 to 2% of a ligand, such as about 1% of a ligand.
   103. The fatty aldehyde purification method of any preceding items, wherein the ligand is a bidentate nitrogen ligand, such as a bidentate nitrogen ligand selected from the group consisting of 2,2′-bipyridine, 4,4′-dimethyl-2,2′-bipyridine, 5,5′-dimethyl-2,2′-bipyridine 2,2′-bipyrimidine, 2,2′-bipyridine-4,4′-dicarboxylic acid or an ester thereof, 2,2′-bipyridine-5,5′-dicarboxylic acid or an ester thereof.
   104. The fatty aldehyde purification method of any preceding items, wherein the crude reaction product further comprises an aminoxyl radical compound, such as 0.01 to 10% of an aminoxyl radical compound, such as 0.01 to 5% of an aminoxyl radical compound, such as about 0.01 to 2% of an aminoxyl radical compound, such as about 0.5% of an aminoxyl radical compound.
   105. The fatty aldehyde purification method of any preceding items, wherein the aminoxyl radical compound is selected from the group consisting of TEMPO, (4-hydroxy-2,2,6,6-tetramethylpiperidin-1-yl)oxyl (4-OH-TEMPO), 4-acetamido-TEMPO, 4-hydroxy-TEMPO benzoate, 4-amino-TEMPO, 2-azaadamantane-N-oxyl, 9-azabicyclo[3.3.1]nonane N-oxyl, 4-carboxy-TEMPO, 4-maleimido-TEMPO, 4-methoxy-TEMPO, 1-methyl-2-azaadamantane-N-oxyl, 4-oxo-TEMPO, and a polymer functionalised with any of said aminoxyl radical compounds.
   106. The fatty aldehyde purification method of any preceding items, wherein the crude reaction product further comprises a base, such as 0.1 to 10% of a base, such as 0.1 to 5% of a base, such as 0.1 to 2% of a base, such as about 0.5% of a base.
   107. The fatty aldehyde purification method of any preceding items, wherein the base is selected from the group consisting of 1-methylimidazole, 1,8-diazabicyclo[5.4.0]undec-7-ene, 1,5-diazabicyclo[4.3.0]non-5-ene, 1,5,7-triazabicyclo[4.4.0]dec-5-ene, 1,1,3,3-tetramethylguanidine, 7-methyl-1,5,7-triazabicyclo[4.4.0]dec-5-ene, and potassium t-butoxide.
   108. The fatty aldehyde purification method of any preceding items, wherein the fatty aldehyde is a saturated fatty aldehyde.
   109. The fatty aldehyde purification method of any preceding items, wherein the fatty aldehyde is a desaturated fatty aldehyde.
   110. The fatty aldehyde purification method of any preceding items, wherein the fatty aldehyde is a C10 to C26 fatty aldehyde.
   111. The fatty aldehyde purification method of any preceding items, wherein the fatty aldehyde is a C10 to C22 fatty aldehyde.
   112. The fatty aldehyde purification method of any preceding items, wherein the fatty aldehyde is a C12 to C20 fatty aldehyde.
   113. The fatty aldehyde purification method of any preceding items, wherein the fatty aldehyde is a C12, C14, or C16 fatty aldehyde.
   114. The fatty aldehyde purification method of any preceding items, wherein the fatty aldehyde is an unbranched fatty aldehyde.
   115. The fatty aldehyde purification method of any preceding items, wherein the fatty aldehyde is selected from the group consisting of:
   (Z)-Δ3 desaturated fatty aldehydes having a carbon chain length of 8, 9, 10, 11, 12, 13, 14, 15, 16, 17, 18, 19, 20, 21 or 22;
   (E)-Δ3 desaturated fatty aldehydes having a carbon chain length of 8, 9, 10, 11, 12, 13, 14, 15, 16, 17, 18, 19, 20, 21 or 22;
   (Z)-Δ5 desaturated fatty aldehydes having a carbon chain length of 8, 9, 10, 11, 12, 13, 14, 15, 16, 17, 18, 19, 20, 21 or 22;
   (E)-Δ5 desaturated fatty aldehydes having a carbon chain length of 8, 9, 10, 11, 12, 13, 14, 15, 16, 17, 18, 19, 20, 21 or 22;
   (Z)-Δ6 desaturated fatty aldehydes having a carbon chain length of 8, 9, 10, 11, 12, 13, 14, 15, 16, 17, 18, 19, 20, 21 or 22;
   (E)-Δ6 desaturated fatty aldehydes having a carbon chain length of 8, 9, 10, 11, 12, 13, 14, 15, 16, 17, 18, 19, 20, 21 or 22;
   (Z)-Δ7 desaturated fatty aldehydes having a carbon chain length of 8, 9, 10, 11, 12, 13, 14, 15, 16, 17, 18, 19, 20, 21 or 22;
   (E)-Δ7 desaturated fatty aldehydes having a carbon chain length of 8, 9, 10, 11, 12, 13, 14, 15, 16, 17, 18, 19, 20, 21 or 22;
   (Z)-Δ8 desaturated fatty aldehydes having a carbon chain length of 9, 10, 11, 12, 13, 14, 15, 16, 17, 18, 19, 20, 21 or 22;
   (E)-Δ8 desaturated fatty aldehydes having a carbon chain length of 9, 10, 11, 12, 13, 14, 15, 16, 17, 18, 19, 20, 21 or 22;
   (Z)-Δ9 desaturated fatty aldehydes having a carbon chain length of 10, 11, 12, 13, 14, 15, 16, 17, 18, 19, 20, 21 or 22; (E)-Δ9 desaturated fatty aldehydes having a carbon chain length of 10, 11, 12, 13, 14, 15, 16, 17, 18, 19, 20, 21 or 22;
   (Z)-Δ10 desaturated fatty aldehydes having a carbon chain length of 11, 12, 13, 14, 15, 16, 17, 18, 19, 20, 21 or 22;
   (E)-Δ10 desaturated fatty aldehydes having a carbon chain length of 11, 12, 13, 14, 15, 16, 17, 18, 19, 20, 21 or 22;
   (Z)-Δ11 desaturated fatty aldehydes having a carbon chain length of 12, 13, 14, 15, 16, 17, 18, 19, 20, 21 or 22;
   (E)-Δ11 desaturated fatty aldehydes having a carbon chain length of 12, 13, 14, 15, 16, 17, 18, 19, 20, 21 or 22;
   (Z)-Δ12 desaturated fatty aldehydes having a carbon chain length of 13, 14, 15, 16, 17, 18, 19, 20, 21 or 22;
   (E)-Δ12 desaturated fatty aldehydes having a carbon chain length of 13, 14, 15, 16, 17, 18, 19, 20, 21 or 22;
   (Z)-Δ13 desaturated fatty aldehydes having a carbon chain length of 14, 15, 16, 17, 18, 19, 20, 21 or 22; and
   (E)-Δ13 desaturated fatty aldehydes having a carbon chain length of 14, 15, 16, 17, 18, 19, 20, 21 or 22.
   116. The fatty aldehyde purification method of any preceding items, wherein the fatty aldehyde is selected from the group consisting of
   (E)7,(Z)9 desaturated fatty aldehydes having a carbon chain length of 10, 11, 12, 13, 14, 15, 16, 17, 18, 19, 20, 21, or 22,
   (E)3,(Z)8,(Z)11 desaturated fatty aldehydes having a carbon chain length of 12, 13, 14, 15, 16, 17, 18, 19, 20, 21, or 22,
   (Z)9,(E)11,(E)13 desaturated fatty aldehydes having a carbon chain length of 14, 15, 16, 17, 18, 19, 20, 21, or 22, and
   (Z11,Z13) desaturated fatty aldehydes having a carbon chain length of 14, 15, 16, 17, 18, 19, 20, 21 or 22,
   (Z9,E12) desaturated fatty aldehydes having a carbon chain length of 13, 14, 15, 16, 17, 18, 19, 20, 21 or 22,
   (7E,9E) desaturated fatty aldehydes having a carbon chain length of 10, 11, 12, 13, 14, 15, 16, 17, 18, 19, 20, 21 or 22, and
   (E8,E10) desaturated fatty aldehydes having a carbon chain length of 11, 12, 13, 14, 15, 16, 17, 18, 19, 20, 21, or 22.
   117. The fatty aldehyde purification method of any preceding items, wherein the fatty aldehyde is selected from the group consisting of:
   (E)7,(Z)9 desaturated fatty aldehyde having a carbon chain length of 14,
   (E)3,(Z)8,(Z)11 desaturated fatty aldehyde having a carbon chain length of 14,
   (Z)9,(E)11,(E)13 desaturated fatty aldehyde having a carbon chain length of 14,
   (E)7,(Z)9 desaturated fatty aldehyde having a carbon chain length of 12,
   (E)3,(Z)8,(Z)11 desaturated fatty aldehyde having a carbon chain length of 12,
   (Z)9,(E)11,(E)13 desaturated fatty aldehyde having a carbon chain length of 12, and
   (E)8,(E)10 desaturated fatty aldehyde having a carbon chain length of 12,
   (E)7,(E)9 desaturated fatty aldehyde having a carbon chain length of 11,
   (Z)11,(Z)13 desaturated fatty aldehyde having a carbon chain length of 16, and
   (Z)9,(E)12 desaturated fatty aldehyde having a carbon chain length of 14.
   118. The fatty aldehyde purification method of any preceding items, wherein the fatty aldehyde is selected from the group consisting of tetradecan-1-al, pentadecan-1-al, hexadecan-1-al, pentadecen-1-al, (Z)-9-hexadecen-1-al, (Z)-11-hexadecen-1-al, (7E,9E)-undeca-7,9-dien-1-al, (11Z, 13Z)-hexadecadien-1-al, (9Z, 12E)-tetradecadien-1-al, and (8E,10E)-dodecadien-1-al.
   119. The fatty aldehyde purification method of any preceding items, wherein the copper ions are copper (II) ions.
   120. The fatty aldehyde purification method of any preceding items, wherein the polar solvent is selected from the group consisting of acetonitrile, dimethylformamide, acetonitrile, propionitrile, butyronitrile, dimethyl sulfoxide, dimethyl acetamide, and propylene carbonate.
   121. The fatty aldehyde purification method of any preceding items, wherein the polar solvent is acetonitrile.
   122. The fatty aldehyde purification method of any preceding items, wherein the apolar, aprotic solvent is selected from the group consisting of linear alkanes, branched alkanes, and cycloalkanes.
   123. The fatty aldehyde purification method of any preceding items, wherein the apolar, aprotic solvent is selected from the group consisting of pentanes, hexanes, heptanes, and octanes.
   124. The fatty aldehyde purification method of any preceding items, wherein the apolar, aprotic solvent is selected from the group consisting of heptane, pentane, hexane, cyclohexane, and octane.
   125. The fatty aldehyde purification method of any preceding items, wherein the acid has a pka value between 3 and 6.
   126. The fatty aldehyde purification method of any preceding items, wherein the acid is a carboxylic acid.
   127. The fatty aldehyde purification method of any preceding items, wherein the carboxylic acid is a C2-C8 carboxylic acid.
   128. The fatty aldehyde purification method of any preceding items, wherein the carboxylic acid is selected from the group consisting of C2-C8 monocarboxylic acids, C2-C8 dicarboxylic acids, and C6-C8 tricarboxylic acids.
   129. The fatty aldehyde purification method of any preceding items, wherein the carboxylic acid is selected from the group consisting of acetic acid, citric acid, propanoic acid, lactic acid, glycolic acid, poly acrylic acid.
   130. The fatty aldehyde purification method of any preceding items, wherein at least 1.0 molar equivalents of carboxylic acid relative to copper is used.
   131. The fatty aldehyde purification method of any preceding items, wherein at least 2.0 molar equivalent of carboxylic acid relative to the copper is used, such as at least 2.4 equivalents.
   132. The fatty aldehyde purification method of any preceding items, wherein the crude reaction product further comprises an oxidising agent and/or a spent oxidising agent.
   133. The fatty aldehyde purification method of any preceding items, wherein the oxidising agent or the spent oxidising agent is selected from the group consisting of TEMPO, (4-hydroxy-2,2,6,6-tetramethylpiperidin-1-yl)oxyl (4-OH-TEMPO), 4-acetamido-TEMPO, 4-hydroxy-TEMPO benzoate, 4-amino-TEMPO, 2-azaadamantane-N-oxyl, 9-azabicyclo[3.3.1]nonane N-oxyl, 4-carboxy-TEMPO, 4-maleimido-TEMPO, 4-methoxy-TEMPO, 1-methyl-2-azaadamantane-N-oxyl, 4-oxo-TEMPO, and a polymer functionalised with any of said aminoxyl radical compounds; or spent agents thereof.
   134. The fatty aldehyde purification method of any preceding items, further comprising a step: evaporating the apolar, aprotic solvent.
   135. The fatty aldehyde purification method of any preceding items, wherein the evaporation of the apolar, aprotic solvent is performed at reduced pressure, such as below 100 mbar, such as below 50 mbar, such as below 40 mbar, such as below 30 mbar.
   136. A method of converting a composition comprising a fatty alcohol to a composition enriched in fatty aldehyde, said method comprising:
   a. converting the composition comprising a fatty alcohol to a composition comprising a fatty aldehyde using the method of any preceding items, and
   b. purifying said composition comprising a fatty aldehyde using the fatty aldehyde purification method of any preceding items, optionally wherein the fatty alcohol and the fatty aldehyde are desaturated.
   137. A composition comprising a fatty aldehyde obtained from the method of any preceding items, optionally wherein the fatty aldehyde is desaturated.
   138. The composition comprising a fatty aldehyde of any preceding item, wherein the composition exhibits an absorption at 680 nm of at most 0.5 in a cuvette having a 5 mm path length.
   139. The composition comprising a fatty aldehyde of any preceding items, wherein the absorption at 680 nm is at most 0.4, such as at most 0.3, such as at most 0.2, such as at most 0.1, such as at most 0.08, such as at most 0.06, such as at most 0.05 in a cuvette having a 5 mm path length.
   140. The composition comprising a fatty aldehyde of any preceding items, wherein the fatty aldehyde composition comprises less than 0.4% copper, such as less than 0.3%, such as 0.2%, such as less than 0.1%, such as less than 0.08%, such as less than 0.06%, such as less than 0.05%, such as less than 0.04%.
   141. A composition comprising more than 93% by weight fatty aldehyde, less than 7% by weight fatty alcohol and less than 2% by weight water.
   142. A method of converting a fatty alcohol to a fatty acetal, said method comprising the steps of:
      a. providing a reaction mixture comprising a fatty alcohol composition comprising the fatty alcohol of any preceding items, a catalyst composition of any preceding items, and a solvent of any preceding items,
      b. exposing the reaction mixture to at least 10 μmol 02 per minute per gram of fatty alcohol by means of bubbling a gas mixture comprising 02 through the reaction mixture, thereby obtaining a fatty aldehyde, and
      c. converting the aldehyde functional groups of the fatty aldehyde to acetal functional groups,
   thereby obtaining the fatty acetal, optionally wherein the fatty alcohol and the fatty acetal are desaturated.
   143. A fatty acetal obtained from the method of any preceding items, optionally wherein the fatty acetal is desaturated.
   144. A fatty aldehyde slow-release composition comprising the fatty acetal of any preceding items.
   145. A method of producing the fatty aldehyde slow-release composition of any preceding items, said method comprising carrying out the method of any preceding items to provide a fatty acetal and formulating said fatty acetal in a slow-release composition optionally wherein the fatty acetal is desaturated.
   146. A method of converting a fatty alcohol to a fatty α-hydroxysulfonic acid, said method comprising the steps of:
      a. providing a reaction mixture comprising a fatty alcohol composition comprising the fatty alcohol of any preceding items, a catalyst composition of any preceding items, and a solvent of any preceding items,
      b. exposing the reaction mixture to at least 10 μmol 02 per minute per gram of fatty alcohol by means of bubbling a gas mixture comprising 02 through the reaction mixture, thereby obtaining a fatty aldehyde, and
      c. converting the aldehyde functional groups of the fatty aldehyde to α-hydroxysulfonic acid functional groups,
   thereby obtaining the fatty α-hydroxysulfonic acid, optionally wherein the fatty alcohol and the fatty α-hydroxysulfonic acid are desaturated.
   147. A fatty α-hydroxysulfonic acid obtained from the method of any preceding items, optionally wherein the fatty α-hydroxysulfonic acid is desaturated.
   148. A fatty aldehyde slow-release composition comprising the fatty α-hydroxysulfonic acid of any preceding items, optionally wherein the fatty α-hydroxysulfonic acid is desaturated.
   149. A method of producing the fatty aldehyde slow-release composition of any preceding items, said method comprising carrying out the method of any preceding items to provide a fatty α-hydroxysulfonic acid and formulating said fatty α-hydroxysulfonic acid into a slow-release composition, thereby obtaining the fatty aldehyde slow-release composition.
   150. A pheromone component produced from renewable feedstocks, said pheromone component having at least than 80% of biobased carbon content.
   151. The pheromone component of any preceding items comprising the fatty aldehyde composition and/or the fatty aldehyde of any preceding items.

1.-24. (canceled)

25. A method for large scale conversion of a fatty alcohol into a fatty aldehyde, said method comprising the steps of:
a) providing a reaction mixture comprising at least 1 kilogram of a fatty alcohol, a catalyst comprising a copper source, at least 1 kilogram of a solvent, and a water absorbing or adsorbing material absorbing or adsorbing water, and
b) dissolving at least 0.01 μmol O₂ per minute per μmol copper in the reaction mixture or at least 0.001 μmol O₂ per minute per μmol initial fatty alcohol in the reaction mixture to the reaction mixture by feeding a gas or a liquid comprising O₂ into the reaction medium and thereby oxidizing more than 50 wt % of the fatty alcohol into fatty aldehyde and less than 50 wt % into fatty acid.

26. The method of claim 25, wherein the method further comprises dissolving at least 0.049 μmol dissolved O₂ per minute per μmol copper in the reaction mixture.

27. The method of claim 25, wherein the method further comprises dissolving:
a) at least 0.0025 μmol dissolved O₂ per minute per μmol initial fatty alcohol in the reaction mixture, or
b) at least 0.025 μmol dissolved O₂ per minute per μmol fatty acid in the reaction mixture.

28. The method of claim 25, wherein the method comprises dissolving at least 10 μmol O₂ per minute per gram of fatty alcohol to the reaction mixture, thereby obtaining the fatty aldehyde.

29. The method of claim 25, wherein the gas or a liquid comprising O₂ is air.

30. The method of claim 25, wherein the feeding of gas or a liquid comprising O₂ into the reaction medium is made by pumping or bubbling a gas or liquid mixture comprising O₂ through the reaction mixture.

31. The method of claim 25, wherein the copper source comprises a copper (I) salt or a combination of copper (II) and a reductant.

32. The method of claim 25, wherein the catalyst further comprises:
a) a ligand which is 2,2′-bipyridine, 4,4′-dimethyl-2,2′-bipyridine, 5,5′-dimethyl-2,2′-bipyridine 2,2′-bipyrimidine, 2,2′-bipyridine-4,4′-dicarboxylic acid or an ester thereof, 2,2′-bipyridine-5,5′-dicarboxylic acid or an ester thereof;
b) an aminoxyl radical compound which is TEMPO, (4-hydroxy-2,2,6,6-tetramethylpiperidin-1-yl)oxyl (4-OH-TEMPO), 4-acetamido-TEMPO, 4-hydroxy-TEMPO benzoate, 4-amino-TEMPO, 2-azaadamantane-N-oxyl, 9-azabicyclo[3.3.1]nonane N-oxyl, 4-carboxy-TEMPO, 4-maleimido-TEMPO, 4-methoxy-TEMPO, 1-methyl-2-azaadamantane-N-oxyl, 4-oxo-TEMPO, or a polymer functionalised with any of said aminoxyl radical compounds; or
c) a base which is 1-methylimidazole, 1,8-diazabicyclo[5.4.0]undec-7-ene, 1,5-diazabicyclo[4.3.0]non-5-ene, 1,5,7-triazabicyclo[4.4.0]dec-5-ene, 1,1,3,3-tetramethylguanidine, 7-methyl-1,5,7-triazabicyclo[4.4.0]dec-5-ene, or potassium t-butoxide.

33. The method of claim 25, wherein the solvent is a non-halogenated solvent.

34. The method of claim 33, wherein the solvent is acetonitrile, dimethyl sulfoxide (DMSO), dimethyl formamide (DMF), pentane, hexane, heptane, cycloalkane, petroleum ether, dioxane, diethyl ether, tetrahydrofuran, ethyl acetate, acetone, nitromethane, propylene carbonate, or a combination thereof.

35. The method of claim 25, wherein the conversion of fatty alcohol to fatty aldehyde is at least 60 wt %.

36. The method of claim 25, wherein:
a) the ratio of fatty acid produced to fatty aldehyde produced is less than 10:90; or
b) the conversion of fatty alcohol to fatty acid is less than 40 wt %.

37. The method of claim 25, wherein the water absorbing or adsorbing material is molecular sieves, silica gels, aluminas, bentonite clays, calcium oxides, alkali metal carbonates, hydrogen carbonates, alkali earth metal carbonates, or a combination thereof.

38. The method of claim 25, wherein the water absorbing or adsorbing material is added to the reaction medium in amounts, so that the water content in the reaction medium after the oxidation process is 2% by weight or less.

39. The method of claim 25, wherein the amount of water absorbing or adsorbing material added is at least 10 g per mmol of fatty alcohol present in the reaction medium prior to oxidation.

40. The method of claim 25, wherein the fatty aldehyde is:
(Z)-Δ3 desaturated fatty aldehydes having a carbon chain length of 8, 9, 10, 11, 12, 13, 14, 15, 16, 17, 18, 19, 20, 21 or 22;
(E)-Δ3 desaturated fatty aldehydes having a carbon chain length of 8, 9, 10, 11, 12, 13, 14, 15, 16, 17, 18, 19, 20, 21 or 22;
(Z)-Δ5 desaturated fatty aldehydes having a carbon chain length of 8, 9, 10, 11, 12, 13, 14, 15, 16, 17, 18, 19, 20, 21 or 22;
(E)-Δ5 desaturated fatty aldehydes having a carbon chain length of 8, 9, 10, 11, 12, 13, 14, 15, 16, 17, 18, 19, 20, 21 or 22;
(Z)-Δ6 desaturated fatty aldehydes having a carbon chain length of 8, 9, 10, 11, 12, 13, 14, 15, 16, 17, 18, 19, 20, 21 or 22;
(E)-Δ6 desaturated fatty aldehydes having a carbon chain length of 8, 9, 10, 11, 12, 13, 14, 15, 16, 17, 18, 19, 20, 21 or 22;
(Z)-Δ7 desaturated fatty aldehydes having a carbon chain length of 8, 9, 10, 11, 12, 13, 14, 15, 16, 17, 18, 19, 20, 21 or 22;
(E)-Δ7 desaturated fatty aldehydes having a carbon chain length of 8, 9, 10, 11, 12, 13, 14, 15, 16, 17, 18, 19, 20, 21 or 22;
(Z)-Δ8 desaturated fatty aldehydes having a carbon chain length of 9, 10, 11, 12, 13, 14, 15, 16, 17, 18, 19, 20, 21 or 22;
(E)-Δ8 desaturated fatty aldehydes having a carbon chain length of 9, 10, 11, 12, 13, 14, 15, 16, 17, 18, 19, 20, 21 or 22;
(Z)-Δ9 desaturated fatty aldehydes having a carbon chain length of 10, 11, 12, 13, 14, 15, 16, 17, 18, 19, 20, 21 or 22;
(E)-Δ9 desaturated fatty aldehydes having a carbon chain length of 10, 11, 12, 13, 14, 15, 16, 17, 18, 19, 20, 21 or 22;
(Z)-Δ10 desaturated fatty aldehydes having a carbon chain length of 11, 12, 13, 14, 15, 16, 17, 18, 19, 20, 21 or 22;
(E)-Δ10 desaturated fatty aldehydes having a carbon chain length of 11, 12, 13, 14, 15, 16, 17, 18, 19, 20, 21 or 22;
(Z)-Δ11 desaturated fatty aldehydes having a carbon chain length of 12, 13, 14, 15, 16, 17, 18, 19, 20, 21 or 22;
(E)-Δ11 desaturated fatty aldehydes having a carbon chain length of 12, 13, 14, 15, 16, 17, 18, 19, 20, 21 or 22;
(Z)-Δ12 desaturated fatty aldehydes having a carbon chain length of 13, 14, 15, 16, 17, 18, 19, 20, 21 or 22;
(E)-Δ12 desaturated fatty aldehydes having a carbon chain length of 13, 14, 15, 16, 17, 18, 19, 20, 21 or 22;
(Z)-Δ13 desaturated fatty aldehydes having a carbon chain length of 14, 15, 16, 17, 18, 19, 20, 21 or 22;
(E)-Δ13 desaturated fatty aldehydes having a carbon chain length of 14, 15, 16, 17, 18, 19, 20, 21 or 22;
(E)7,(Z)9 desaturated fatty aldehydes having a carbon chain length of 10, 11, 12, 13, 14, 15, 16, 17, 18, 19, 20, 21, or 22,
(E)3,(Z)8,(Z)11 desaturated fatty aldehydes having a carbon chain length of 12, 13, 14, 15, 16, 17, 18, 19, 20, 21, or 22,
(Z)9,(E)11,(E)13 desaturated fatty aldehydes having a carbon chain length of 14, 15, 16, 17, 18, 19, 20, 21, or 22,
(Z)11,(Z)13 desaturated fatty aldehydes having a carbon chain length of 14, 15, 16, 17, 18, 19, 20, 21 or 22,
(Z)9,(E)12 desaturated fatty aldehydes having a carbon chain length of 13, 14, 15, 16, 17, 18, 19, 20, 21 or 22,
(E)7,(E)9 desaturated fatty aldehydes having a carbon chain length of 10, 11, 12, 13, 14, 15, 16, 17, 18, 19, 20, 21 or 22,
(E)8,(E)10 desaturated fatty aldehydes having a carbon chain length of 11, 12, 13, 14, 15, 16, 17, 18, 19, 20, 21, or 22,
(E)7,(Z)9 desaturated fatty aldehyde having a carbon chain length of 14,
(E)3,(Z)8,(Z)11 desaturated fatty aldehyde having a carbon chain length of 14,
(Z)9,(E)11,(E)13 desaturated fatty aldehyde having a carbon chain length of 14,
(E)7,(Z)9 desaturated fatty aldehyde having a carbon chain length of 12,
(E)3,(Z)8,(Z)11 desaturated fatty aldehyde having a carbon chain length of 12,
(Z)9,(E)11,(E)13 desaturated fatty aldehyde having a carbon chain length of 12,
(E)8,(E)10 desaturated fatty aldehyde having a carbon chain length of 12
(E)7,(E)9 desaturated fatty aldehyde having a carbon chain length of 11,
(Z)11,(Z)13 desaturated fatty aldehyde having a carbon chain length of 16,
(Z)9,(E)12 desaturated fatty aldehyde having a carbon chain length of 14, tetradecan-1-al, pentadecan-1-al, hexadecan-1-al, pentadecen-1-al, (Z)-9-hexadecen-1-al, (Z)-11-hexadecen-1-al, (7E,9E)-undeca-7,9-dien-1-al, (11Z, 13Z)-hexadecadien-1-al, (9Z,12E)-tetradecadien-1-al, or (8E,10E)-dodecadien-1-al.

41. The method of claim 25, further comprising steps for purifying the fatty aldehyde comprising:
a) providing a purification mixture comprising:
i. the fatty aldehyde,
ii. copper ions, and
iii. a polar solvent;
b) mixing said purification mixture with an apolar, aprotic solvent and an acid to create an extraction mixture comprising an apolar phase and a polar phase allowing the fatty aldehyde to be extracted from the polar phase to the apolar phase and
c) separating the apolar phase comprising purified aldehyde from the polar phase.

42. The method of claim 41, wherein:
a) the purification mixture comprises 0.05 to 5.0 wt % copper ions;
b) the acid is a C2-C8 monocarboxylic acid, a C2-C8 dicarboxylic acids, or a C6-C8 tricarboxylic acids; or
c) at least 2.0 molar equivalent of the acid relative to the copper ions is used.

43. A composition comprising more than 93% by weight fatty aldehyde, less than 7% by weight fatty alcohol and less than 2% by weight water.

44. The composition of claim 43, wherein the light absorption at 680 nm is at most 0.4 in a cuvette having a 5 mm path length.
